(19) United States
(12) Patent Application Publication
(10) Pub. No.: US 2020/0384188 A1
(43) Pub. Date: Dec. 10, 2020

(54) SYSTEMS, DEVICES, FORMULATIONS AND METHODS FOR CONTROLLED DRUG DELIVERY

(71) Applicant: BEXSON BIOMEDICAL, INC., Santa Barbara, CA (US)

(72) Inventors: Jeffrey BECKER, Santa Barbara, CA (US); Gregg PETERSON, Santa Barbara, CA (US); Jason WALLACH, Santa Barbara, CA (US)

(21) Appl. No.: 16/763,933

(22) PCT Filed: Nov. 14, 2018
(86) PCT No.: PCT/US18/61121

§ 371 Date: May 13, 2020

Related U.S. Application Data
(60) Provisional application No. 62/586,024, filed on Nov. 14, 2017.
(60) Provisional application No. 62/679,630, filed on Jun. 1, 2018.

(51) Int. Cl. A61M 5/142; A61M 5/50; A61K 31/136; A61K 31/137; A61K 47/24; A61K 47/40; A61K 9/00

(52) U.S. Cl. CPC A61M 5/142; A61M 5/5086; A61K 31/136; A61K 31/137; A61M 2205/276; A61K 47/40; A61K 9/0019; A61M 2205/502; A61M 2005/14268; A61K 47/24

(57) ABSTRACT

Provided herein are systems, devices, formulations, and methods for treating a subject for treating, preventing, or ameliorating at least one symptom of a disorder, disease, or condition according to a controlled dosage regimen to provide effective treatment while reducing the risk of side effects or abuse.

### CROSS-REFERENCE

This application claims the benefit of U.S. Provisional Application No. 62/586,024, filed Nov. 14, 2017, and U.S. Provisional Application No. 62/679,630, filed Jun. 1, 2018, which applications are incorporated herein by reference.

### BACKGROUND

Ketamine is an NMDA receptor antagonist that has found use in treating pain and depression and numerous other psychiatric and physical disorders. However, some of ketamine's side effects, poor bioavailability, formulation and pharmacokinetics limit delivery options and render it susceptible to abuse and addiction. These factors and risks associated with off-label ketamine use present a challenge to effective treatment.

### SUMMARY

The standard of care for treating physical, neurological and psychiatric disorders with ketamine HCl typically involves in-office use with intravenous delivery being the most common delivery method. Intravenous delivery can require significant monitoring efforts, IV placement, and various other mechanical requirements of IV procedure (e.g., normal saline, sedatives such as midazolam or propofol, hospital bed or IV chair). Some practitioners deliver ketamine in the office through intramuscular delivery, which generally requires reduced medical paraphernalia (e.g., no IV, saline bags, cannulas, etc., needed). Depending upon the procedural protocol and practice patterns of a given practitioner, usually in this situation there is reduced monitoring as well, often consisting only of intermittent blood pressure and pulse, and/or pulse oximetry. Alternatively, ketamine is sometimes administered outside the office or clinic in the form of sublingual, compounded “troches,” oral compounded capsules, and intra-nasal compounded spray. Each of these three modes of ketamine delivery has various drawbacks that can limit their use depending upon the clinical parameters of a given medical case.

The systems, devices, kits, formulations, and methods disclosed herein provide an innovative solution to an ongoing problem in the treatment of physical, neurological and psychiatric disorders with various drugs such as, for example, ketamine. Ketamine is administered in the hospital or clinic by or under the supervision of a doctor, nurse, or other medical practitioner. Because intravenous or intramuscular delivery currently requires the presence of the medical practitioner, patients need to make frequent trips to the hospital or clinic to receive regular doses. This challenge is compounded by the short half-life of ketamine, which requires continuous or frequent administration to maintain an effective plasma concentration. Furthermore, increasing the dosing interval necessitates increasing the bolus that is administered with each dose, which creates a risk of addiction and/or abuse of an active ingredient such as ketamine.

Accordingly, one advantage provided by the systems, devices, kits, formulations, and methods disclosed herein includes providing at-home delivery of drug formulations such as ketamine, optionally by intramuscular or subcutaneous delivery. Intramuscular and subcutaneous drug administration bypasses various problems with traditional at-home treatments such as oral capsules and nasal sprays. Oral, sublingual and nasal delivery requires higher dosages than intramuscular or subcutaneous delivery to achieve comparable clinical effects, which carries with it a risk for bladder dysfunction and inflammation (i.e. cystitis) due to higher exposure to metabolites with higher dosing. Oral or sublingual administration is also often unreliable due to the presence of food or chyme in the stomach or proximal small intestines, decreasing absorption, and substantial first pass metabolism and substantial variability in first pass metabolism. Intranasal administration can precipitate allergic rhinitis, epistaxis (nosebleeds), and/or bacterial or viral sinusitis. Rectal administration is inconvenient and can be negatively affected by hemorrhoids or diarrhea. Moreover, even with effective in-clinic treatment, many patients are unable to make regular clinic visits to receive the treatment they need. The costs (opportunity and direct) associated with repeated in-office ketamine treatments and post treatment monitoring pose a significant barrier to effective treatment. This is especially true in large urban centers where it can be particularly difficult for patients to arrange for the time and help needed to travel to and from treatment centers given that patients are not permitted to drive after treatments. Effective at-home treatment with ketamine would notably reduce this barrier to treatment for patients that suffer from physical, neurological and psychiatric conditions for which ketamine is effective (e.g., CRPS, pain, depression, suicidality) throughout the country. Patients would not need clinic visits with the frequency required by current standard of care if they have access to safe and effective at-home delivery systems and methods. Such convenience can save time and money for the patient, the physician, and for the reimbursing entity. Therefore, the systems, devices, kits, formulations, and methods disclosed herein can combine the advantages of intramuscular or subcutaneous delivery with the convenience and cost effectiveness of at-home treatment. This can decrease the procedural burden and medical equipment required during treatment in the clinic or hospital preformed through the current art consisting of IV infusion or IM bolus injection.

Another advantage provided by the systems, devices, kits, formulations, and methods disclosed herein are treatment regimens that provide effective treatment while reducing the risk of side effect(s) and/or dissociative symptom(s) associated with standard of care treatments. Such treatment regimens can provide more frequent administration of smaller doses and/or one or more sustained doses, which can partially or completely mitigate the extreme side effects that often arise from administration of a large bolus. For example, delivering at a sustained lower dose and/or a lower infusion rate can mitigate much of the uncomfortable psychological or dissociative side effects associated with higher doses, and reduces or eliminates the recovery time required for a patient to re-engage their lives in comparison to higher dose IV or IM injections performed in-clinic. Moreover, at-home dosage regimens disclosed herein can provide low but effective steady state plasma concentrations outside of the clinic setting, which is unfeasible under current standard of care at home for certain active ingredients such as ketamine due to its short half-life and substantial first pass metabolism.

Another advantage provided by the systems, devices, kits, formulations, and methods disclosed herein is clinic or home administration of one or more doses of a drug such as ketamine according to a programmed dosage regimen within established safe parameters administered using a drug delivery device. Such devices allow drug administration in the traditional hospital or clinic setting, but also provide the option to self-administer at home or outside the hospital/clinic setting. A doctor or healthcare provider can program a delivery device with a dosage regimen, and the patient or subject is able to use the device to self-administer one or more doses at home. The subject is thus given limited control to implement the pre-programmed dosage regimen. The use of the pre-programmed dosage regimen to self-medicate outside of the clinic allows empiric discovery and/or accurate titration of blood levels to minimal effective dose ranges of an active ingredient such as ketamine. This can decrease the procedural burden and medical equipment required during treatment in the clinic or hospital preformed through the current art consisting of IV infusion or IM bolus injection. Another benefit of finding a minimal effective dose and reducing unnecessary metabolite exposure can be a reduction in same-day and next-days side effects that are associated with current in-office and at-home treatment such as dissociation, disorientation, confusion, drowsiness, brain fog and physical fatigue. Moreover, the dosage regimen can be programmed to control the rate of drug delivery to mitigate certain side effects such as, for example, adverse cardiac effects associated with higher doses of ketamine. In some cases, the dosage regimen is programmed for sustained release and/or extended release dosing of a drug such as ketamine.

Another advantage provided by the systems, devices, kits, formulations, and methods disclosed herein can include the prevention of administration of a bolus of a drug such as ketamine beyond a dosage limit. Administration of a large bolus of a drug such as ketamine can invoke effects such as dissociation, disorientation, confusion, drowsiness, increased heart rate, elevated blood pressure, euphoria, and even temporary paralysis. Limiting the maximum dosage of a drug prevents the subject from exceeding the limits of a set dosing regimen, abusing the drug, or overdosing.

Another advantage provided by the systems, devices, kits, formulations, and methods disclosed herein is tamper resistant drug delivery that discourages or prevents unauthorized access to the drug stored within the device and/or a drug cartridge. Drugs such as ketamine can be subject to abuse, and delivery mechanisms that cede control to the patient are accompanied by the risk of abuse and/or addiction. Tamper resistant devices and/or drug cartridges help prevent unauthorized access to the drug formulation contained within, thereby limiting use of the drug to authorized uses such as according to a pre-programmed dosage regimen.

Another advantage provided by the systems, devices, kits, formulations, and methods disclosed herein is improved subcutaneous or intramuscular tissue tolerance to, and by extension improved efficacy with, the ketamine formulation through the addition of excipients designed to increased pH to a more tolerable range, and/or complexing ketamine in solution and tissue and/or addressing hypertonicity. Subcutaneously delivered ketamine has been described as irritating to local tissues, and even causing sterile abscess at the injection site. This is likely due to acidic pH (3.5-5.5) and/or hypertonicity associated with current formulations. The ketamine formulation disclosed herein can improve tissue tolerance making extended periods of subcutaneous infusion more tolerable and therefor, also, more effective. Generally, increased tolerability decreases treatment dropout rates, thereby increasing the overall efficacy as per intent-to-treat analysis.

Another advantage provided by the systems, devices, kits, formulations, and methods disclosed herein is “real-time” recording of pain status through pain schedule self-reporting via pain scales in combination with medication formulation dosing history. Patient reporting of pain status after the fact is notoriously inaccurate and difficult to use in medical charting, decisions and clinical treatment planning. The systems, devices, kits, formulations, and methods disclosed herein can provide a novel and notable advantage in the clinical art of pain treatment by providing more accurate reporting on pain status and the efficacy of treatments than is currently possible through soliciting history from the patient in the office visit. There are significant advantages to a system that can describe nuances in a patient's pain cycle that might otherwise be lost. For example, reporting of recorded of pain schedule data might reveal patterning to pain flares that can point toward changes in dosing regimen and/or behavioral approaches that can increase success (e.g., going to bed earlier in repeated late night flares, changing jobs if physicality at work flares a patient consistently by the afternoon). In some embodiments, the dosing regimen is adjusted to allow increased dosage during periods of expected pain flares or increased pain. For example, this can be accomplished by increasing a preset maximum dosage threshold for the time of day when the patient's pain schedule data indicates increased pain or discomfort. Accordingly, the patient or user may request or enter an increased dosage beyond the normal maximum threshold during these time periods when self-administering the drug formulation.

Another advantage provided by the systems, devices, kits, formulations, and methods disclosed herein is to adjust the formulation strength to address the realities of wearable pump reservoir sizing and mechanics. Current formulations of ketamine can be incompatible with the pump mechanics and reservoir sizes specifically with respect to the total dosing range required for some individuals over 1 to 3 days. Changing the strength of the formulation can address that limitation and can increase the efficacy of treatment.

In some aspects, disclosed herein is a drug delivery device comprising: a) a pump mechanism configured for administering a drug formulation comprising an NMDA receptor modulator or antagonist; and b) a user interface allowing a subject to select and self-administer a dose of the drug formulation from a selection of at least one pre-programmed dosage regimen that is not configurable by the subject; wherein the at least one dosage regimen provides an effective clinical response or an established effective drug plasma concentration. In some embodiments, the at least one dosage regimen provides an effective steady state drug plasma concentration. In some embodiments, the at least one dosage regimen provides an effective Cₘₐₓ drug plasma concentration. In some embodiments, the at least one dosage regimen is locked after configuration by an authorized user to deter modification by the subject. In some embodiments, the at least one dosage regimen is locked after configuration by the manufacturer to deter modification by the patient. In some embodiments, the drug delivery device is configured to be tamper-proof to deter administration of a dose of the drug formulation that deviates from the at least one dosage regimen. In some embodiments, the drug delivery device is configured to be tamper-resistant to deter administration of a dose of the drug formulation that deviates from the at least one dosage regimen. In some embodiments, the drug formulation is stored in tamper-proof cartridge. In some embodiments, the drug formulation is stored in tamper-resistant cartridge. In some embodiments, the drug formulation is stored in sealed cartridge. In some embodiments, the drug delivery device comprises a reservoir for storing the drug formulation prior to administration. In some embodiments, the drug delivery device comprises a tamper-proof reservoir for storing the drug formulation prior to administration. In some embodiments, the drug delivery device comprises a tamper-resistant reservoir for storing the drug formulation prior to administration. In some embodiments, the drug delivery device comprises a sealed reservoir for storing the drug formulation prior to administration. In some embodiments, the drug formulation is stored in tamper-resistant cartridge inserted only by a pharmacist, a doctor or the manufacturer. In some embodiments, the drug delivery device comprises a sealed reservoir for storing the drug formulation prior to administration. In some embodiments, the drug delivery device comprises a sealed reservoir filled only by a pharmacist, a doctor or the manufacturer for storing the drug formulation prior to administration. In some embodiments, the drug delivery device comprises drug reservoir for storing the drug formulation prior to administration that is not sealed itself but is sealed within the larger device to discourage tampering. In some embodiments, the at least one dosage regimen reduces side effects of the drug formulation while providing effective drug plasma concentration. In some embodiments, the side effects comprise drug dependence or addiction. In some embodiments, the side effects comprise hallucination, disorientation, dissociation, dizziness, drowsiness, increased heart rate, elevated blood pressure, nausea, vomiting, fatigue, brain fog, confusion, anxiety, distress, shortness of breath, or any combination thereof. In some embodiments, the drug delivery device deters abuse of the drug formulation by limiting control of the at least one dosage regimen by the subject. In some embodiments, the drug delivery device is configured to administer the drug formulation according to the at least one dosage regimen for treating Treatment Resistant Depression. In some embodiments, the drug delivery device is configured to administer the drug formulation according to the at least one dosage regimen for treating chronic pain. In some embodiments, the drug delivery device is configured to administer the drug formulation according to the at least one dosage regimen for treating acute pain. In some embodiments, the drug delivery device is configured to administer the drug formulation according to the at least one dosage regimen for chronic regional pain syndrome (CRPS). In some embodiments, the drug delivery device is configured to administer the drug formulation according to the at least one dosage regimen for treating pain associated with Ehlers-Danlos Syndrome. In some embodiments, the drug delivery device is configured to administer the drug formulation according to the at least one dosage regimen for treating post laminectomy syndrome. In some embodiments, the drug delivery device is configured to administer the drug formulation according to the at least one dosage regimen for treating pain associated with post laminectomy syndrome. In some embodiments, the drug delivery device is configured to administer the drug formulation according to the at least one dosage regimen for treating failed back syndrome. In some embodiments, the drug delivery device is configured to administer the drug formulation according to the at least one dosage regimen for treating pain associated with failed back syndrome. In some embodiments, the drug delivery device is configured to administer the drug formulation according to the at least one dosage regimen for treating post-operative pain. In some embodiments, the drug delivery device is configured to administer the drug formulation according to the at least one dosage regimen for treating diabetic neuropathy. In some embodiments, the drug delivery device is configured to administer the drug formulation according to the at least one dosage regimen for treating major depressive disorder, treatment resistant major depressive disorder, suicidality, suicidal ideation, dysthymia or persistent depressive disorder, bipolar depressive disorder type I, bipolar depressive disorder type II, chronic pain, eating disorder NOS, pain disorder NOS, panic disorder, post-traumatic stress disorder, obsessive-compulsive disorder, complex regional pain syndrome, reflex sympathetic dystrophy, or any combination thereof. In some embodiments, the NMDA receptor antagonist is ketamine or a pharmaceutically acceptable salt thereof. In some embodiments, the NMDA receptor antagonist is an arylcyclohexylamine or arylcyclohexylamine derivative. In some embodiments, the NMDA receptor antagonist also acts as a dopamine reuptake inhibitor, μ-opioid receptor agonist, σ receptor agonist, nACh receptor antagonist, D2 receptor agonistic, or any combination thereof. In some embodiments, the NMDA receptor antagonist is ketamine, phencyclidine (PCP), 3-MeO-Phencylidine, 4-MeO-Phencyclidine, eticyclidine (PCE), 3-MeO-PCE, methoxetamine (MXE), tiletamine, or tenocyclidine (TCP). In some embodiments, the drug formulation comprises a second active ingredient for mitigating side effects of the NMDA receptor antagonist. In some embodiments, the second active ingredient is a benzodiazepine, a selective serotonin 5-HT3 receptor antagonist, or a beta-blocker. In some embodiments, the drug formulation comprises a second active ingredient for altering pharmacokinetic properties of the NMDA receptor antagonist. In some embodiments, the second active ingredient is an inhibitor of CYP2B6 and/or CYP3A and/or CYP2C9. In some embodiments, the drug formulation comprises a pharmaceutically acceptable excipient for complexing the NMDA receptor antagonist. In some embodiments, the drug formulation comprises a pharmaceutically acceptable excipient for emulsifying mixed ionic and non-ionic forms of the NMDA receptor antagonist. In some embodiments, the drug formulation comprises a pharmaceutically acceptable excipient for buffering the solution containing the NMDA receptor antagonist. In some embodiments, the drug formulation comprises a pharmaceutically acceptable excipient for adjusting the pH of the solution containing the NMDA receptor antagonist. In some embodiments, the at least one dosage regimen is configured by an authorized user who is a healthcare provider for the subject. In some embodiments, the at least one dosage regimen is prescribed for the subject by a healthcare provider. In some embodiments, the subject is not authorized to configure or modify the at least one dosage regimen. In some embodiments, the drug delivery device allows limited modification of the at least one dosage regimen by the subject. In some embodiments, the at least one dosage regimen comprises a plurality of dosing options selectable by the subject. In some embodiments, the plurality of dosing options is selected from the group consisting of bolus injection, and/or continuous infusion. In some embodiments, the plurality of dosing options comprises differences in dosage size, dosage rate, infusion duration, or any combination thereof. In some embodiments, the drug delivery device further comprises a remote access module allowing an authorized user to remotely configure or modify the at least one dosage regimen over a network. In some embodiments, the drug delivery device is configured to communicate with a user communication device. In some embodiments, the user communication device is configured to enable user control of the drug delivery device. In some embodiments, the user communication device comprises a communication module providing instructions to the drug delivery device and/or receiving data from the drug delivery device (e.g., usage data, self-rated pain schedules). In some embodiments, the drug delivery device and/or an associated user communication device comprises a data module storing information from self-rated pain schedules completed by the subject. In some embodiments, the drug delivery device and/or an associated user communication device comprises a user interface allowing the subject or user to enter self-rated pain schedule(s). In some embodiments, the user is prompted to enter information for a pain schedule. In some embodiments, the drug delivery device and/or an associated user communication device comprises a data module storing information from pain schedules completed by the subject that can be downloaded for physician review. In some embodiments, the drug delivery device and/or an associated user communication device comprises a data module storing information from pain schedules completed by the subject that can be included in dosage selection and control. In some embodiments, the drug delivery device and/or an associated user communication device comprises a data module storing information for doses administered by the subject. In some embodiments, the drug delivery device further comprises a monitoring module allowing an authorized user to remotely monitor the at least one dosage regimen over a network. In some embodiments, the drug delivery device further comprises a communications module allowing the subject to send a request to an authorized user regarding the at least one dosage regimen over a network. In some embodiments, the drug delivery device further comprises a communications module allowing the drug delivery device to send and receive information over a network. In some embodiments, the drug delivery device further comprises a communications module allowing the drug delivery device to pair with a communications device that provides a network connection for communicating with an authorized user. In some embodiments, the at least one dosage regimen comprises a dosage limit setting an upper limit on a size of the dose. In some embodiments, the drug delivery device prohibits administration of a dose of the drug formulation that exceeds a dosage limit. In some embodiments, the drug delivery device prohibits administration of a dose of the drug formulation that causes a total daily dose to exceed a daily dosage limit. In some embodiments, the drug delivery device prohibits administration of a dose of the drug formulation at an infusion rate that exceeds a dosage limit. In some embodiments, the drug delivery device deters abuse of the drug formulation. In some embodiments, the pump mechanism is configured to administer the drug formulation through subcutaneous or intramuscular injection. In some embodiments, the dose comprises an infusion rate of at least about 0.1 mg/hour. In some embodiments, the dose comprises an infusion rate of no more than about 200 mg/hour. In some embodiments, the dose comprises an infusion rate from about 0.1 mg/hour to about 200 mg/hour. In some embodiments, the dose comprises an infusion of at least about ten (10) minutes. In some embodiments, the dose comprises an infusion that is continuous. In some embodiments, the dose comprises an infusion rate of at least 0.1 mg/hour for at least ten (10) minutes. In some embodiments, the dose comprises an infusion rate of at least 1 mg/hour for at least ten (10) minutes. In some embodiments, the NMDA receptor antagonist is a racemic mixture of ketamine. In some embodiments, the NMDA receptor antagonist is substantially pure S-ketamine. In some embodiments, the NMDA receptor antagonist is substantially pure R-ketamine. In some embodiments, the dosage regimen provides a clinically effective steady-state concentration of the NMDA receptor antagonist outside of a hospital or clinical setting. In some embodiments, the dosage regimen provides a clinically effective steady-state concentration of the NMDA receptor antagonist for at least 1 week. In some embodiments, the dosage regimen provides a clinically effective steady-state concentration of the NMDA receptor antagonist for at least 1 day. In some embodiments, the dosage regimen provides a clinically effective steady-state concentration of the NMDA receptor antagonist for at least 1 hour. In some embodiments, the dosage regimen provides an average treatment steady state plasma concentration of at least 1 ng/mL with a peak trough fluctuation of no more than 100% of the average steady state plasma concentration during treatment. In some embodiments, the at least one dosage regimen provides a clinically effective steady-state concentration of the NMDA receptor antagonist with peak trough fluctuation of no more than 100% while the steady-state plasma concentration is maintained. In some embodiments, the at least one dosage regimen provides a clinically effective steady-state concentration of the NMDA receptor antagonist with a Cₘₐₓ to Cₘᵢₙ ratio of no more than 4. In some embodiments, the at least one dosage regimen provides a concentration of the NMDA receptor antagonist of at least 1 ng/mL throughout a duration of the at least one dosage regimen. In some embodiments, the at least one dosage regimen comprises at least 1 dose per month. In some embodiments, the at least one dosage regimen comprises a single continuous dose. In some embodiments, the at least one dosage regimen comprises a loading dose and a series of maintenance doses. In some embodiments, the at least one dosage regimen comprises periodic doses. In some embodiments, the at least one dosage regimen comprises aperiodic doses. In some embodiments, the device is configured to administer a pharmaceutical formulation according to the dosage regimen for treating, preventing, or ameliorating at least one symptom of a disorder, disease, or condition. In some embodiments, the disorder, disease, or condition is a mental or psychiatric disorder, a neurological condition or disorder, a physical disorder, pain, or an inflammatory disorder. In some embodiments, the disorder, disease, or condition is pain. In some embodiments, the neurological condition or disorder is chronic pain. In some embodiments, the disorder, disease, or condition is a mental or psychiatric disorder. In some embodiments, the mental or psychiatric disorder is Major Depressive Disorder, treatment resistant major depressive disorder, suicidality, suicidal ideation, Substance-Related Disorder, Sedative-, Hypnotic-, or Anxiolytic-Related Disorder, Sedative-, hypnotic-, or anxiolytic withdrawal, alcohol withdrawal, cannabis dependence, cannabis withdrawal, barbiturate dependence, barbiturate withdrawal, benzodiazepine dependence, benzodiazepine withdrawal, amphetamine dependence, amphetamine withdrawal, opioid dependence, opioid withdrawal, opioid-related disorder, alcohol dependence, cocaine dependence, or cocaine withdrawal.

In some aspects, disclosed herein is a system comprising: a) a drug delivery device comprising a pump mechanism for administering a drug formulation comprising an NMDA receptor antagonist and a user interface allowing a subject to self-administer a dose of the drug formulation from a selection of at least one pre-programmed dosage regimen that is not configurable by the subject; and b) a digital device of an authorized user in communication with the drug delivery device to allow the authorized user to configure, modify, or monitor the dosage regimen; wherein the at least one dosage regimen provides an effective steady state drug plasma concentration while reducing side effects. In some embodiments, the at least one dosage regimen provides an effective steady state drug plasma concentration. In some embodiments, the at least one dosage regimen provides an effective Cₘₐₓ drug plasma concentration. In some embodiments, the at least one dosage regimen is locked after configuration by an authorized user to deter modification by the subject. In some embodiments, the at least one dosage regimen is locked after configuration by the manufacturer to deter modification by the patient. In some embodiments, the drug delivery device is configured to be tamper-proof to deter administration of a dose of the drug formulation that deviates from the at least one dosage regimen. In some embodiments, the drug delivery device is configured to be tamper-resistant to deter administration of a dose of the drug formulation that deviates from the at least one dosage regimen. In some embodiments, the drug formulation is stored in tamper-proof cartridge. In some embodiments, the drug formulation is stored in tamper-resistant cartridge. In some embodiments, the drug formulation is stored in sealed cartridge. In some embodiments, the drug delivery device comprises a reservoir for storing the drug formulation prior to administration. In some embodiments, the drug delivery device comprises a tamper-proof reservoir for storing the drug formulation prior to administration. In some embodiments, the drug delivery device comprises a tamper-resistant reservoir for storing the drug formulation prior to administration. In some embodiments, the drug delivery device comprises a sealed reservoir for storing the drug formulation prior to administration. In some embodiments, the drug formulation is stored in tamper-resistant cartridge inserted only by a pharmacist, a doctor or the manufacturer. In some embodiments, the drug delivery device comprises a sealed reservoir for storing the drug formulation prior to administration. In some embodiments, the drug delivery device comprises a sealed reservoir filled only by a pharmacist, a doctor or the manufacturer for storing the drug formulation prior to administration. In some embodiments, the drug delivery device comprises drug reservoir for storing the drug formulation prior to administration that is not sealed itself but is sealed within the larger device to discourage tampering. In some embodiments, the at least one dosage regimen reduces side effects of the drug formulation while providing effective drug plasma concentration. In some embodiments, the side effects comprise drug dependence or addiction. In some embodiments, the side effects comprise hallucination, disorientation, dissociation, dizziness, drowsiness, increased heart rate, elevated blood pressure, nausea, vomiting, fatigue, brain fog, confusion, anxiety, distress, shortness of breath or any combination thereof. In some embodiments, the drug delivery device deters abuse of the drug formulation by limiting control of the at least one dosage regimen by the subject. In some embodiments, the drug delivery device is configured to administer the drug formulation according to the at least one dosage regimen for treating Treatment Resistant Depression. In some embodiments, the drug delivery device is configured to administer the drug formulation according to the at least one dosage regimen for treating chronic pain. In some embodiments, the drug delivery device is configured to administer the drug formulation according to the at least one dosage regimen for treating acute pain. In some embodiments, the drug delivery device is configured to administer the drug formulation according to the at least one dosage regimen for chronic regional pain syndrome. In some embodiments, the drug delivery device is configured to administer the drug formulation according to the at least one dosage regimen for treating pain associated with Ehlers-Danlos Syndrome. In some embodiments, the drug delivery device is configured to administer the drug formulation according to the at least one dosage regimen for treating post laminectomy syndrome. In some embodiments, the drug delivery device is configured to administer the drug formulation according to the at least one dosage regimen for treating pain associated with post laminectomy syndrome. In some embodiments, the drug delivery device is configured to administer the drug formulation according to the at least one dosage regimen for treating failed back syndrome. In some embodiments, the drug delivery device is configured to administer the drug formulation according to the at least one dosage regimen for treating pain associated with failed back syndrome. In some embodiments, the drug delivery device is configured to administer the drug formulation according to the at least one dosage regimen for treating post-operative pain. In some embodiments, the drug delivery device is configured to administer the drug formulation according to the at least one dosage regimen for treating diabetic neuropathy. In some embodiments, the drug delivery device is configured to administer the drug formulation according to the at least one dosage regimen for treating major depressive disorder, treatment resistant major depressive disorder, suicidality, suicidal ideation, dysthymia or persistent depressive disorder, bipolar depressive disorder type I, bipolar depressive disorder type II, chronic pain, eating disorder NOS, pain disorder NOS, panic disorder, post-traumatic stress disorder, obsessive-compulsive disorder, complex regional pain syndrome, reflex sympathetic dystrophy, or any combination thereof. In some embodiments, the NMDA receptor antagonist is ketamine or a pharmaceutically acceptable salt thereof. In some embodiments, the NMDA receptor antagonist is an arylcyclohexylamine or arylcyclohexylamine derivative. In some embodiments, the NMDA receptor antagonist also acts as a dopamine reuptake inhibitor, μ-opioid receptor agonist, σ receptor agonist, nACh receptor antagonist, D2 receptor agonistic, or any combination thereof. In some embodiments, the NMDA receptor antagonist is ketamine, phencyclidine (PCP), 3-MeO-Phencylidine, 4-MeO-Phencyclidine, eticyclidine (PCE), 3-MeO-PCE, methoxetamine (MXE), tiletamine, or tenocyclidine (TCP). In some embodiments, the drug formulation comprises a second active ingredient for mitigating side effects of the NMDA receptor antagonist. In some embodiments, the second active ingredient is a benzodiazepine, a selective serotonin 5-HT3 receptor antagonist, or a beta-blocker. In some embodiments, the drug formulation comprises a second active ingredient for altering pharmacokinetic properties of the NMDA receptor antagonist. In some embodiments, the second active ingredient is an inhibitor of CYP2B6 and/or CYP3A and/or CYP2C9. In some embodiments, the drug formulation comprises a pharmaceutically acceptable excipient for complexing the NMDA receptor antagonist. In some embodiments, the drug formulation comprises a pharmaceutically acceptable excipient for emulsifying mixed ionic and non-ionic forms of the NMDA receptor antagonist. In some embodiments, the drug formulation comprises a pharmaceutically acceptable excipient for buffering the solution containing the NMDA receptor antagonist. In some embodiments, the drug formulation comprises a pharmaceutically acceptable excipient for adjusting the pH of the solution containing the NMDA receptor antagonist. In some embodiments, the at least one dosage regimen is configured by an authorized user who is a healthcare provider for the subject. In some embodiments, the at least one dosage regimen is prescribed for the subject by a healthcare provider. In some embodiments, the subject is not authorized to configure or modify the at least one dosage regimen. In some embodiments, the drug delivery device allows limited modification of the at least one dosage regimen by the subject. In some embodiments, the at least one dosage regimen comprises a plurality of dosing options selectable by the subject. In some embodiments, the plurality of dosing options is selected from the group consisting of bolus injection, and/or continuous infusion. In some embodiments, the plurality of dosing options comprises differences in dosage size, dosage rate, infusion duration, or any combination thereof. In some embodiments, the drug delivery device further comprises a remote access module allowing an authorized user to remotely configure or modify the at least one dosage regimen over a network. In some embodiments, the drug delivery device is configured to communicate with a user communication device. In some embodiments, the user communication device is configured to enable user control of the drug delivery device. In some embodiments, the user communication device comprises a communication module providing instructions to the drug delivery device and/or receiving data from the drug delivery device (e.g., usage data, self-rated pain schedules). In some embodiments, the drug delivery device and/or an associated user communication device comprises a data module storing information from self-rated pain schedules completed by the subject. In some embodiments, the drug delivery device and/or an associated user communication device comprises a user interface allowing the subject or user to enter self-rated pain schedule(s). In some embodiments, the user is prompted to enter information for a pain schedule. In some embodiments, the drug delivery device and/or an associated user communication device comprises a data module storing information from pain schedules completed by the subject that can be downloaded for physician review. In some embodiments, the drug delivery device and/or an associated user communication device comprises a data module storing information from pain schedules completed by the subject that can be included in dosage selection and control. In some embodiments, the drug delivery device and/or an associated user communication device comprises a data module storing information for doses administered by the subject. In some embodiments, the drug delivery device further comprises a monitoring module allowing an authorized user to remotely monitor the at least one dosage regimen over a network. In some embodiments, the drug delivery device further comprises a communications module allowing the subject to send a request to an authorized user regarding the at least one dosage regimen over a network. In some embodiments, the drug delivery device further comprises a communications module allowing the drug delivery device to send and receive information over a network. In some embodiments, the drug delivery device further comprises a communications module allowing the drug delivery device to pair with a communications device that provides a network connection for communicating with an authorized user. In some embodiments, the at least one dosage regimen comprises a dosage limit setting an upper limit on a size of the dose. In some embodiments, the drug delivery device prohibits administration of a dose of the drug formulation that exceeds a dosage limit. In some embodiments, the drug delivery device prohibits administration of a dose of the drug formulation that causes a total daily dose to exceed a daily dosage limit. In some embodiments, the drug delivery device prohibits administration of a dose of the drug formulation at an infusion rate that exceeds a dosage limit. In some embodiments, the drug delivery device deters abuse of the drug formulation. In some embodiments, the pump mechanism is configured to administer the drug formulation through subcutaneous or intramuscular injection. In some embodiments, the dose comprises an infusion rate of at least about 0.1 mg/hour. In some embodiments, the dose comprises an infusion rate of no more than about 200 mg/hour. In some embodiments, the dose comprises an infusion rate from about 0.1 mg/hour to about 200 mg/hour. In some embodiments, the dose comprises an infusion of at least about ten (10) minutes. In some embodiments, the dose comprises an infusion that is continuous. In some embodiments, the dose comprises an infusion rate of at least 0.1 mg/hour for at least ten (10) minutes. In some embodiments, the dose comprises an infusion rate of at least 1 mg/hour for at least ten (10) minutes. In some embodiments, the NMDA receptor antagonist is a racemic mixture of ketamine. In some embodiments, the NMDA receptor antagonist is substantially pure S-ketamine. In some embodiments, the NMDA receptor antagonist is substantially pure R-ketamine. In some embodiments, the dosage regimen provides a clinically effective steady-state concentration of the NMDA receptor antagonist outside of a hospital or clinical setting. In some embodiments, the dosage regimen provides a clinically effective steady-state concentration of the NMDA receptor antagonist for at least 1 week. In some embodiments, the dosage regimen provides a clinically effective steady-state concentration of the NMDA receptor antagonist for at least 1 day. In some embodiments, the dosage regimen provides a clinically effective steady-state concentration of the NMDA receptor antagonist for at least 1 hour. In some embodiments, the dosage regimen provides an average treatment steady state plasma concentration of at least 1 ng/mL with a peak trough fluctuation of no more than 100% of the average steady state plasma concentration during treatment. In some embodiments, the at least one dosage regimen provides a clinically effective steady-state concentration of the NMDA receptor antagonist with peak trough fluctuation of no more than 100% while the steady-state plasma concentration is maintained. In some embodiments, the at least one dosage regimen provides a clinically effective steady-state concentration of the NMDA receptor antagonist with a Cₘₐₓ to Cₘᵢₙ ratio of no more than 4. In some embodiments, the at least one dosage regimen provides a concentration of the NMDA receptor antagonist of at least 1 ng/mL throughout a duration of the at least one dosage regimen. In some embodiments, the at least one dosage regimen comprises at least 1 dose per month. In some embodiments, the at least one dosage regimen comprises a single continuous dose. In some embodiments, the at least one dosage regimen comprises a loading dose and a series of maintenance doses. In some embodiments, the at least one dosage regimen comprises periodic doses. In some embodiments, the at least one dosage regimen comprises aperiodic doses. In some embodiments, the device is configured to administer a pharmaceutical formulation according to the dosage regimen for treating, preventing, or ameliorating at least one symptom of a disorder, disease, or condition. In some embodiments, the disorder, disease, or condition is a mental or psychiatric disorder, a neurological condition or disorder, a physical disorder, pain, or an inflammatory disorder. In some embodiments, the disorder, disease, or condition is pain. In some embodiments, the neurological condition or disorder is chronic pain. In some embodiments, the disorder, disease, or condition is a mental or psychiatric disorder. In some embodiments, the mental or psychiatric disorder is Major Depressive Disorder, treatment resistant major depressive disorder, suicidality, suicidal ideation, Substance-Related Disorder, Sedative-, Hypnotic-, or Anxiolytic-Related Disorder, Sedative-, hypnotic-, or anxiolytic withdrawal, alcohol withdrawal, cannabis dependence, cannabis withdrawal, barbiturate dependence, barbiturate withdrawal, benzodiazepine dependence, benzodiazepine withdrawal, amphetamine dependence, amphetamine withdrawal, opioid dependence, opioid withdrawal, opioid-related disorder, alcohol dependence, cocaine dependence, or cocaine withdrawal.

In some aspects, disclosed herein is a method for self-treatment by a subject outside of a hospital or clinical setting, comprising: a) obtaining a drug delivery device for administering a dose of a drug formulation comprising an NMDA receptor modulator or NMDA receptor antagonist; and b) self-administering the dose from a selection of at least one pre-programmed dosage regimen that is not configurable by the subject; wherein the at least one dosage regimen provides an effective steady state drug plasma concentration while reducing side effects. In some embodiments, the at least one dosage regimen provides an effective steady state drug plasma concentration. In some embodiments, the at least one dosage regimen provides an effective Cₘₐₓ drug plasma concentration. In some embodiments, the at least one dosage regimen is locked after configuration by an authorized user to deter modification by the subject. In some embodiments, the at least one dosage regimen is locked after configuration by the manufacturer to deter modification by the patient. In some embodiments, the drug delivery device is configured to be tamper-proof to deter administration of a dose of the drug formulation that deviates from the at least one dosage regimen. In some embodiments, the drug delivery device is configured to be tamper-resistant to deter administration of a dose of the drug formulation that deviates from the at least one dosage regimen. In some embodiments, the drug formulation is stored in tamper-proof cartridge. In some embodiments, the drug formulation is stored in tamper-resistant cartridge. In some embodiments, the drug formulation is stored in sealed cartridge. In some embodiments, the drug delivery device comprises a reservoir for storing the drug formulation prior to administration. In some embodiments, the drug delivery device comprises a tamper-proof reservoir for storing the drug formulation prior to administration. In some embodiments, the drug delivery device comprises a tamper-resistant reservoir for storing the drug formulation prior to administration. In some embodiments, the drug delivery device comprises a sealed reservoir for storing the drug formulation prior to administration. In some embodiments, the drug formulation is stored in tamper-resistant cartridge inserted only by a pharmacist, a doctor or the manufacturer. In some embodiments, the drug delivery device comprises a sealed reservoir for storing the drug formulation prior to administration. In some embodiments, the drug delivery device comprises a sealed reservoir filled only by a pharmacist, a doctor or the manufacturer for storing the drug formulation prior to administration. In some embodiments, the drug delivery device comprises drug reservoir for storing the drug formulation prior to administration that is not sealed itself but is sealed within the larger device to discourage tampering. In some embodiments, the at least one dosage regimen reduces side effects of the drug formulation while providing effective drug plasma concentration. In some embodiments, the side effects comprise drug dependence or addiction. In some embodiments, the side effects comprise hallucination, disorientation, dissociation, dizziness, drowsiness, increased heart rate, elevated blood pressure, nausea, vomiting, fatigue, brain fog, confusion, anxiety, distress, shortness of breath or any combination thereof. In some embodiments, the drug delivery device deters abuse of the drug formulation by limiting control of the at least one dosage regimen by the subject. In some embodiments, the drug delivery device is configured to administer the drug formulation according to the at least one dosage regimen for treating Treatment Resistant Depression. In some embodiments, the drug delivery device is configured to administer the drug formulation according to the at least one dosage regimen for treating chronic pain. In some embodiments, the drug delivery device is configured to administer the drug formulation according to the at least one dosage regimen for treating acute pain. In some embodiments, the drug delivery device is configured to administer the drug formulation according to the at least one dosage regimen for chronic regional pain syndrome. In some embodiments, the drug delivery device is configured to administer the drug formulation according to the at least one dosage regimen for treating pain associated with Ehlers-Danlos Syndrome. In some embodiments, the drug delivery device is configured to administer the drug formulation according to the at least one dosage regimen for treating post laminectomy syndrome. In some embodiments, the drug delivery device is configured to administer the drug formulation according to the at least one dosage regimen for treating pain associated with post laminectomy syndrome. In some embodiments, the drug delivery device is configured to administer the drug formulation according to the at least one dosage regimen for treating failed back syndrome. In some embodiments, the drug delivery device is configured to administer the drug formulation according to the at least one dosage regimen for treating pain associated with failed back syndrome. In some embodiments, the drug delivery device is configured to administer the drug formulation according to the at least one dosage regimen for treating post-operative pain. In some embodiments, the drug delivery device is configured to administer the drug formulation according to the at least one dosage regimen for treating diabetic neuropathy. In some embodiments, the drug delivery device is configured to administer the drug formulation according to the at least one dosage regimen for treating major depressive disorder, treatment resistant major depressive disorder, suicidality, suicidal ideation, dysthymia or persistent depressive disorder, bipolar depressive disorder type I, bipolar depressive disorder type II, chronic pain, eating disorder NOS, pain disorder NOS, panic disorder, post-traumatic stress disorder, obsessive-compulsive disorder, complex regional pain syndrome, reflex sympathetic dystrophy, or any combination thereof. In some embodiments, the NMDA receptor antagonist is ketamine or a pharmaceutically acceptable salt thereof. In some embodiments, the NMDA receptor antagonist is an arylcyclohexylamine or arylcyclohexylamine derivative. In some embodiments, the NMDA receptor antagonist also acts as a dopamine reuptake inhibitor, μ-opioid receptor agonist, σ receptor agonist, nACh receptor antagonist, D2 receptor agonistic, or any combination thereof. In some embodiments, the NMDA receptor antagonist is ketamine, phencyclidine (PCP), 3-MeO-Phencylidine, 4-MeO-Phencyclidine, eticyclidine (PCE), 3-MeO-PCE, methoxetamine (MXE), tiletamine, or tenocyclidine (TCP). In some embodiments, the drug formulation comprises a second active ingredient for mitigating side effects of the NMDA receptor antagonist. In some embodiments, the second active ingredient is a benzodiazepine, a selective serotonin 5-HT3 receptor antagonist, or a beta-blocker. In some embodiments, the drug formulation comprises a second active ingredient for altering pharmacokinetic properties of the NMDA receptor antagonist. In some embodiments, the second active ingredient is an inhibitor of CYP2B6 and/or CYP3A and/or CYP2C9. In some embodiments, the drug formulation comprises a pharmaceutically acceptable excipient for complexing the NMDA receptor antagonist. In some embodiments, the drug formulation comprises a pharmaceutically acceptable excipient for emulsifying mixed ionic and non-ionic forms of the NMDA receptor antagonist. In some embodiments, the drug formulation comprises a pharmaceutically acceptable excipient for buffering the solution containing the NMDA receptor antagonist. In some embodiments, the drug formulation comprises a pharmaceutically acceptable excipient for adjusting the pH of the solution containing the NMDA receptor antagonist. In some embodiments, the at least one dosage regimen is configured by an authorized user who is a healthcare provider for the subject. In some embodiments, the at least one dosage regimen is prescribed for the subject by a healthcare provider. In some embodiments, the subject is not authorized to configure or modify the at least one dosage regimen. In some embodiments, the drug delivery device allows limited modification of the at least one dosage regimen by the subject. In some embodiments, the at least one dosage regimen comprises a plurality of dosing options selectable by the subject. In some embodiments, the plurality of dosing options is selected from the group consisting of bolus injection, and/or continuous infusion. In some embodiments, the plurality of dosing options comprises differences in dosage size, dosage rate, infusion duration, or any combination thereof. In some embodiments, the drug delivery device further comprises a remote access module allowing an authorized user to remotely configure or modify the at least one dosage regimen over a network. In some embodiments, the drug delivery device is configured to communicate with a user communication device. In some embodiments, the user communication device is configured to enable user control of the drug delivery device. In some embodiments, the user communication device comprises a communication module providing instructions to the drug delivery device and/or receiving data from the drug delivery device (e.g., usage data, self-rated pain schedules). In some embodiments, the drug delivery device and/or an associated user communication device comprises a data module storing information from self-rated pain schedules completed by the subject. In some embodiments, the drug delivery device and/or an associated user communication device comprises a user interface allowing the subject or user to enter self-rated pain schedule(s). In some embodiments, the user is prompted to enter information for a pain schedule. In some embodiments, the drug delivery device and/or an associated user communication device comprises a data module storing information from pain schedules completed by the subject that can be downloaded for physician review. In some embodiments, the drug delivery device and/or an associated user communication device comprises a data module storing information from pain schedules completed by the subject that can be included in dosage selection and control. In some embodiments, the drug delivery device and/or an associated user communication device comprises a data module storing information for doses administered by the subject. In some embodiments, the drug delivery device further comprises a monitoring module allowing an authorized user to remotely monitor the at least one dosage regimen over a network. In some embodiments, the drug delivery device further comprises a communications module allowing the subject to send a request to an authorized user regarding the at least one dosage regimen over a network. In some embodiments, the drug delivery device further comprises a communications module allowing the drug delivery device to send and receive information over a network. In some embodiments, the drug delivery device further comprises a communications module allowing the drug delivery device to pair with a communications device that provides a network connection for communicating with an authorized user. In some embodiments, the at least one dosage regimen comprises a dosage limit setting an upper limit on a size of the dose. In some embodiments, the drug delivery device prohibits administration of a dose of the drug formulation that exceeds a dosage limit. In some embodiments, the drug delivery device prohibits administration of a dose of the drug formulation that causes a total daily dose to exceed a daily dosage limit. In some embodiments, the drug delivery device prohibits administration of a dose of the drug formulation at an infusion rate that exceeds a dosage limit. In some embodiments, the drug delivery device deters abuse of the drug formulation. In some embodiments, the pump mechanism is configured to administer the drug formulation through subcutaneous or intramuscular injection. In some embodiments, the dose comprises an infusion rate of at least about 0.1 mg/hour. In some embodiments, the dose comprises an infusion rate of no more than about 200 mg/hour. In some embodiments, the dose comprises an infusion rate from about 0.1 mg/hour to about 200 mg/hour. In some embodiments, the dose comprises an infusion of at least about ten (10) minutes. In some embodiments, the dose comprises an infusion that is continuous. In some embodiments, the dose comprises an infusion rate of at least 0.1 mg/hour for at least ten (10) minutes. In some embodiments, the dose comprises an infusion rate of at least 1 mg/hour for at least ten (10) minutes. In some embodiments, the NMDA receptor antagonist is a racemic mixture of ketamine. In some embodiments, the NMDA receptor antagonist is substantially pure S-ketamine. In some embodiments, the NMDA receptor antagonist is substantially pure R-ketamine. In some embodiments, the dosage regimen provides a clinically effective steady-state concentration of the NMDA receptor antagonist outside of a hospital or clinical setting. In some embodiments, the dosage regimen provides a clinically effective steady-state concentration of the NMDA receptor antagonist for at least 1 week. In some embodiments, the dosage regimen provides a clinically effective steady-state concentration of the NMDA receptor antagonist for at least 1 day. In some embodiments, the dosage regimen provides a clinically effective steady-state concentration of the NMDA receptor antagonist for at least 1 hour. In some embodiments, the dosage regimen provides an average treatment steady state plasma concentration of at least 1 ng/mL with a peak trough fluctuation of no more than 100% of the average steady state plasma concentration during treatment. In some embodiments, the at least one dosage regimen provides a clinically effective steady-state concentration of the NMDA receptor antagonist with peak trough fluctuation of no more than 100% while the steady-state plasma concentration is maintained. In some embodiments, the at least one dosage regimen provides a clinically effective steady-state concentration of the NMDA receptor antagonist with a Cₘₐₓ to Cₘᵢₙ ratio of no more than 4. In some embodiments, the at least one dosage regimen provides a concentration of the NMDA receptor antagonist of at least 1 ng/mL throughout a duration of the at least one dosage regimen. In some embodiments, the at least one dosage regimen comprises at least 1 dose per month. In some embodiments, the at least one dosage regimen comprises a single continuous dose. In some embodiments, the at least one dosage regimen comprises a loading dose and a series of maintenance doses. In some embodiments, the at least one dosage regimen comprises periodic doses. In some embodiments, the at least one dosage regimen comprises aperiodic doses. In some embodiments, the device is configured to administer a pharmaceutical formulation according to the dosage regimen for treating, preventing, or ameliorating at least one symptom of a disorder, disease, or condition. In some embodiments, the disorder, disease, or condition is a mental or psychiatric disorder, a neurological condition or disorder, a physical disorder, pain, or an inflammatory disorder. In some embodiments, the disorder, disease, or condition is pain. In some embodiments, the neurological condition or disorder is chronic pain. In some embodiments, the disorder, disease, or condition is a mental or psychiatric disorder. In some embodiments, the mental or psychiatric disorder is Major Depressive Disorder, treatment resistant major depressive disorder, suicidality, suicidal ideation, Substance-Related Disorder, Sedative-, Hypnotic-, or Anxiolytic-Related Disorder, Sedative-, hypnotic-, or anxiolytic withdrawal, alcohol withdrawal, cannabis dependence, cannabis withdrawal, barbiturate dependence, barbiturate withdrawal, benzodiazepine dependence, benzodiazepine withdrawal, amphetamine dependence, amphetamine withdrawal, opioid dependence, opioid withdrawal, opioid-related disorder, alcohol dependence, cocaine dependence, or cocaine withdrawal.

In some aspects, disclosed herein is a method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is not configurable by the subject; wherein the dosage regimen provides an average ketamine plasma concentration of at least 1 ng/mL with a peak trough fluctuation of no more than 100%.

In some aspects, disclosed herein is a drug delivery device comprising: a) a reservoir for storing a drug formulation comprising ketamine; b) an infusion pump connected to the reservoir and configured for subcutaneous infusion of the drug formulation; and c) a user interface enabling a subject to self-administer a dose of the drug formulation according to a pre-programmed dosage regimen that is configurable only by an authorized user; wherein the dosage regimen provides an average ketamine plasma concentration of at least 1 ng/mL with a peak trough fluctuation of no more than 100%.

In some aspects, disclosed herein is a system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user; and b) a digital device of an authorized user in communication with the drug delivery device to allow the authorized user to configure, modify, or monitor the dosage regimen; wherein the dosage regimen provides an average ketamine plasma concentration of at least 1 ng/mL with a peak trough fluctuation of no more than 100%.

In some aspects, disclosed herein is a method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is not configurable by the subject; wherein the dosage regimen comprises periodic doses that provide a clinically effective ketamine plasma concentration with a peak trough fluctuation of no more than 100%.

In some aspects, disclosed herein is a drug delivery device comprising: a) a reservoir for storing a drug formulation comprising ketamine; b) an infusion pump connected to the reservoir and configured for subcutaneous infusion of the drug formulation; and c) a user interface enabling a subject to self-administer a dose of the drug formulation according to a pre-programmed dosage regimen that is configurable only by an authorized user; wherein the dosage regimen comprises periodic doses that provide a clinically effective ketamine plasma concentration with a peak trough fluctuation of no more than 100%.

In some aspects, disclosed herein is a system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user; and b) a digital device of an authorized user in communication with the drug delivery device to allow the authorized user to configure, modify, or monitor the dosage regimen; wherein the dosage regimen comprises periodic doses that provide a clinically effective ketamine plasma concentration with a peak trough fluctuation of no more than 100%.

In some aspects, disclosed herein is a method for self-treatment by a subject outside of a hospital or clinical setting, comprising: a) obtaining a drug delivery device for administering a dose of a drug formulation comprising an NMDA receptor antagonist; and b) self-administering the dose according to a pre-programmed dosage regimen that is not configurable by the subject; wherein the drug delivery device is programmed to restrict administration of a bolus of the drug formulation that exceeds a pre-programmed dosage limit.

In some aspects, disclosed herein is a drug delivery device comprising: a) a receptacle for receiving a cartridge storing a drug formulation comprising ketamine; b) an infusion pump connected to the receptacle and configured for subcutaneous infusion of the drug formulation; and c) a user interface enabling a subject to self-administer a dose of the drug formulation according to a pre-programmed dosage regimen that is configurable only by an authorized user; wherein the drug delivery device is programmed to restrict administration of a bolus of the drug formulation that exceeds a pre-programmed dosage limit.

In some aspects, disclosed herein is a system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user; and b) a digital device of an authorized user in communication with the drug delivery device to allow the authorized user to configure, modify, or monitor the dosage regimen; wherein the drug delivery device is programmed to restrict administration of a bolus of the drug formulation that exceeds a pre-programmed dosage limit.

In some aspects, disclosed herein is a method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is configurable only by an authorized user who is not the subject; wherein the drug delivery device is programmed to allow at-home administration of the drug formulation, wherein the drug delivery device is configured to be tamper-proof to deter the subject from deviating from the pre-programmed dosage regimen.

In some aspects, disclosed herein is a drug delivery device comprising: a) a receptacle for receiving a cartridge storing a drug formulation comprising ketamine; b) an infusion pump connected to the receptacle and configured for subcutaneous infusion of the drug formulation; and c) a user interface enabling a subject to self-administer a dose of the drug formulation according to a pre-programmed dosage regimen that is configurable only by an authorized user who is not the subject; wherein the drug delivery device is programmed to allow at-home administration of the drug formulation, wherein the drug delivery device is configured to be tamper-proof to deter the subject from deviating from the pre-programmed dosage regimen.

In some aspects, disclosed herein is a system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user who is not the subject; and b) a digital device of an authorized user in communication with the drug delivery device to allow the authorized user to configure, modify, or monitor the dosage regimen; wherein the drug delivery device is programmed to allow at-home administration of the drug formulation, wherein the drug delivery device is configured to be tamper-proof to deter the subject from deviating from the pre-programmed dosage regimen.

In some aspects, disclosed herein is a method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is configurable only by an authorized user who is not the subject; wherein the drug delivery device restricts access to the drug formulation to deter usage that deviates from the pre-programmed dosage regimen.

In some aspects, disclosed herein is a drug delivery device comprising: a) a reservoir for storing a drug formulation comprising ketamine; b) an infusion pump connected to the reservoir and configured for subcutaneous infusion of the drug formulation; and c) a user interface enabling a subject to self-administer a dose of the drug formulation according to a pre-programmed dosage regimen that is configurable only by an authorized user who is not the subject; wherein the drug delivery device restricts access to the drug formulation to deter usage that deviates from the pre-programmed dosage regimen.

In some aspects, disclosed herein is a system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user who is not the subject; and b) a digital device of an authorized user in communication with the drug delivery device to allow the authorized user to configure, modify, or monitor the dosage regimen; wherein the drug delivery device restricts access to the drug formulation to deter usage that deviates from the pre-programmed dosage regimen.

In some aspects, disclosed herein is a method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is configurable only by an authorized user who is not the subject; wherein the dosage regimen provides a plasma concentration of ketamine that continuously remains is no lower than a minimum effective concentration and below a minimum toxic concentration for at least 1 week.

In some aspects, disclosed herein is a drug delivery device comprising: a) a storage chamber storing drug formulation comprising ketamine; b) an infusion pump connected to the reservoir and configured for subcutaneous infusion of the drug formulation; and c) a user interface enabling a subject to self-administer a dose of the drug formulation according to a pre-programmed dosage regimen that is configurable only by an authorized user who is not the subject; wherein the dosage regimen provides a plasma concentration of ketamine that continuously remains is no lower than a minimum effective concentration and below a minimum toxic concentration for at least 1 week.

In some aspects, disclosed herein is a system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user who is not the subject; and b) a digital device of an authorized user in communication with the drug delivery device to allow the authorized user to configure, modify, or monitor the dosage regimen; wherein the dosage regimen provides a plasma concentration of ketamine that continuously remains is no lower than a minimum effective concentration and below a minimum toxic concentration for at least 1 week.

In some aspects, disclosed herein is a method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is configurable only by an authorized user who is not the subject; wherein the dosage regimen comprises an initial loading dose and a series of maintenance doses to maintain an effective plasma concentration of ketamine.

In some aspects, disclosed herein is a drug delivery device comprising: a) a storage chamber storing drug formulation comprising ketamine; b) an infusion pump connected to the reservoir and configured for subcutaneous infusion of the drug formulation; and c) a user interface enabling a subject to self-administer a dose of the drug formulation and according to a pre-programmed dosage regimen that is configurable only by an authorized user who is not the subject; wherein the dosage regimen comprises an initial loading dose and a series of maintenance doses to maintain an effective plasma concentration of ketamine.

In some aspects, disclosed herein is a system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user who is not the subject; and b) a digital device of an authorized user in communication with the drug delivery device to allow the authorized user to configure, modify, or monitor the dosage regimen; wherein the dosage regimen comprises an initial loading dose and a series of maintenance doses to maintain an effective plasma concentration of ketamine.

In some aspects, disclosed herein is a method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is configurable only by an authorized user who is not the subject; wherein the dosage regimen comprises at least 3 doses a week to maintain an effective plasma concentration of ketamine through at-home administration of the drug formulation.

In some aspects, disclosed herein is a drug delivery device comprising: a) a storage chamber storing a drug formulation comprising ketamine; b) an infusion pump connected to the reservoir and configured for subcutaneous infusion of the drug formulation; and c) a user interface enabling a subject to self-administer a dose of the drug formulation and according to a pre-programmed dosage regimen that is configurable only by an authorized user who is not the subject; wherein the dosage regimen comprises at least 3 doses a week to maintain an effective plasma concentration of ketamine through at-home administration of the drug formulation.

In some aspects, disclosed herein is a system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user who is not the subject; and b) a digital device of an authorized user in communication with the drug delivery device to allow the authorized user to configure, modify, or monitor the dosage regimen; wherein the dosage regimen comprises at least 3 doses a week to maintain an effective plasma concentration of ketamine through at-home administration of the drug formulation.

In some aspects, disclosed herein is a method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is configurable only by an authorized user who is not the subject; wherein the dosage regimen allows the subject to reach a steady state plasma concentration of ketamine with a peak trough fluctuation percentage of no more than 30% within one day of initiating the dosage regimen.

In some aspects, disclosed herein is a drug delivery device comprising: a) a storage chamber storing a drug formulation comprising ketamine; b) an infusion pump connected to the reservoir and configured for subcutaneous infusion of the drug formulation; and c) a user interface enabling a subject to self-administer a dose of the drug formulation and according to a pre-programmed dosage regimen that is configurable only by an authorized user who is not the subject; wherein the dosage regimen allows the subject to reach a steady state plasma concentration of ketamine with a peak trough fluctuation percentage of no more than 30% within one day of initiating the dosage regimen.

In some aspects, disclosed herein is a system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user who is not the subject; and b) a digital device of an authorized user in communication with the drug delivery device to allow the authorized user to configure, modify, or monitor the dosage regimen; wherein the dosage regimen allows the subject to reach a steady state plasma concentration of ketamine with a peak trough fluctuation percentage of no more than 30% within one day of initiating the dosage regimen.

In some aspects, disclosed herein is a method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is not configurable by the subject; wherein the dosage regimen allows the subject to reach a steady state plasma concentration of ketamine with a peak concentration no greater than 100% of a trough concentration for at least one week.

In some aspects, disclosed herein is a drug delivery device comprising: a) a pump mechanism configured for subcutaneous delivery of a drug formulation comprising ketamine; and b) a user interface enabling a subject to self-administer a dose of the drug formulation and according to a pre-programmed dosage regimen that is not configurable by the subject; wherein the dosage regimen allows the subject to reach a steady state plasma concentration of ketamine with a peak concentration no greater than 100% of a trough concentration for at least one week.

In some aspects, disclosed herein is a system comprising: a) a drug delivery device comprising a pump mechanism for administering a drug formulation comprising ketamine and a user interface allowing a subject to self-administer a dose of the drug formulation according to a pre-programmed dosage regimen that is not configurable by the subject; and b) a digital device of an authorized user in communication with the drug delivery device to allow the authorized user to configure, modify, or monitor the dosage regimen; wherein the dosage regimen allows the subject to reach a plasma concentration of ketamine with a peak concentration no greater than 100% of a trough concentration for at least one week.

In some aspects, disclosed herein is a formulation for administration according to the systems and methods described herein. In some embodiments, the formulation is configured for subcutaneous administration. In some embodiments, the formulation is an aqueous, injectable formulation.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 schematically illustrates a computer control system of a drug delivery device that is programmed or otherwise configured to implement methods provided herein.

FIG. 2 schematically illustrates the components of a sealed delivery authentication and pump control system that is programmed or otherwise configured to implement methods provided herein.

FIG. 3 schematically illustrates a delivery authentication and pump control system with a tamper proof delivery cartridge and a sealed delivery system.

FIG. 4 schematically illustrates the patient verification and authentication pathway for the drug delivery device disclosed herein.

FIG. 5 schematically illustrates the two-point authentication pathway interfacing between the patient screen and the physician screen. The patient controlled menu is displayed on the left hand pathway. The physician order menus are displayed on the right hand pathway.

FIG. 6 shows a diagram of an automatic cannulation device (spring driven and motor driven, respectively).

FIG. 7 shows a diagram of a replaceable delivery module.

FIG. 8 shows an illustration of the communication links between a wearable device, a user communication or mobile device, and the cloud in one embodiment of the present disclosure.

FIG. 9A, FIG. 9B, FIG. 9C, and FIG. 9D show embodiments of pain schedules and queries for evaluating patient pain level.

FIG. 10 shows a graph of the mean reaction time on tail flick following subcutaneous infusion of ketamine (50 mg/kg).

FIG. 11 shows a graph of the percentage change from baseline on tail flick following subcutaneous infusion of ketamine (50 mg/kg).

FIG. 12 shows a graph of the mean force applied on Randall Selitto following subcutaneous infusion of ketamine (50 mg/kg).

FIG. 13 shows a graph of the mean force applied on Randall Selitto following subcutaneous infusion of ketamine (50 mg/kg).

FIG. 14 shows a graph of the percentage change in Randall Selitto following subcutaneous infusion of ketamine (50 mg/kg).

FIG. 15 shows the mean concentration-time profiles of ketamine in pig plasma.

FIG. 16 shows dose normalized Cmax and AUC0-24 relationships of ketamine in pig plasma.

FIG. 17 shows a table of the individual body weights of and doses administered to male and female minipigs given subcutaneous infusion doses of ketamine.

FIG. 18A and FIG. 18B show tables of the individual and mean concentrations (ng/mL) of ketamine in pig plasma following a single subcutaneous infusion administration.

FIG. 19 shows a table summarizing the mean pharmacokinetic parameters for ketamine in pig plasma following a single subcutaneous infusion administration.

FIG. 20A and FIG. 20B show tables of individual and mean pharmacokinetic parameters for ketamine in pig plasma following a single subcutaneous infusion administration.

FIG. 21 shows a table of dose proportionality ratios for ketamine Cₘₐₓ and AUC₀₋₂₄ in pig plasma following a single subcutaneous infusion administration.

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

Disclosed herein are systems, devices, and methods for administering one or more doses of a drug formulation such as a ketamine formulation according to one or more pre-programmed dosage regimen. The dose is often administered by subcutaneous or intravenous injection using a programmable delivery device. The delivery device can allow for treatment both in the clinic or hospital setting under supervision of a healthcare provider or via self-administration at home. A doctor or healthcare provider is able to program the delivery device with one or more dosage regimen(s) and optionally sets dosage limits or other limits on the subject's ability to alter the dose and/or dosage regimen(s). The dosage regimen(s) can include selectable dosage options that give the subject limited control over the dose. The device is usually configured to be tamper resistant to prevent unauthorized access to the drug formulation stored on the device. Alternatively or in combination, the drug formulation is stored in a tamper resistant cartridge or vessel that is operably connected to the delivery device. In some cases, the device is remotely programmable to enable a doctor or healthcare provider to configure or modify the dosage regimen(s) via a network connection without requiring the subject to travel to the clinic or hospital. Self-administration of the drug formulation according to the pre-programmed dosage regimen(s) can allow an effective plasma concentration of the active ingredient to be reached and maintained outside of the clinic setting and without requiring large bolus infusions. Accordingly, plasma concentration fluctuation may be reduced compared to standard of care treatments at home.

In some embodiments, the systems, devices, kits, formulations, and methods disclosed herein help mitigate one or more side effect(s) of the main active ingredient and/or metabolites thereof. In some embodiments, the systems, devices, kits, formulations, and methods disclosed herein help mitigate the side effect(s) of ketamine administration for treating physical, neurological and psychiatric disorder(s). In some embodiments, a side effect of ketamine includes hallucination, disorientation, dissociation, dizziness, drowsiness, increased heart rate, elevated blood pressure, nausea, vomiting, fatigue, brain fog, confusion, anxiety, distress, shortness of breath.

In some embodiments, the systems, devices, kits, formulations, and methods disclosed herein are used to administer a drug formulation comprising an NMDA receptor antagonist. In an exemplary embodiment, the NMDA receptor antagonist is ketamine. In some embodiments, the NMDA receptor antagonist is an arylcyclohexylamine or arylcyclohexylamine derivative. In some embodiments, the NMDA receptor antagonist is a dopamine reuptake inhibitor, μ-opioid receptor agonist, σ receptor agonist, nACh receptor antagonist, a substance P antagonists (SPA), a neurokinin 1 (NK1) receptor antagonist, or D2 receptor agonistic. In some embodiments, the NMDA receptor antagonist is ketamine, phencyclidine (PCP), 3-MeO-Phencylidine, 4-MeO-Phencyclidine, eticyclidine (PCE), 3-MeO-PCE, methoxetamine (MXE), tiletamine, or tenocyclidine (TCP).

In some embodiments, the drug formulation comprises a second or additional active ingredient. In some embodiments, the second active ingredient mitigates one or more side effects of the active ingredient such as ketamine. In some embodiments, the second active ingredient is a benzodiazepine (e.g., lorazepam, midazolam), a selective serotonin 5-HT3 receptor antagonist (e.g., ondansetron), or a beta-blocker (e.g., propranolol, atenolol). In some embodiments, the second active ingredient alters the pharmacokinetic properties of the active ingredient (such as an NMDA inhibitor like ketamine). In some embodiments, the second active ingredient is an inhibitor of CYP2B6 and/or CYP3A and/or CYP2C9. In some embodiments, an inhibitor of CYP2B6 is clopidogrel, ticlopidine, orphenadrine, candesartan, amlodipine, felodipine, memantine, clotrimazole, voriconazole, azelastine, clopidogrel, clofibrate, fenofibrate, 2-phenyl-2-(1-piperidinyl)propane, resveratrol, alpha-viniferin, epsilon-viniferin or pregabalin. In some embodiments, an inhibitor of CYP3A is nefazodone, aprepitant, fluvoxamine, itraconazole, verapamil, orphenadrine, bergamottin, mibefradil, ketoconazole, itraconazole, resveratrol, alpha-viniferin, epsilon-viniferin or diltiazem.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The term “subject,” as used herein, generally refers to a human. The subject can be a healthy individual, an individual that has or is suspected of having a disease or a pre-disposition to the disease, or an individual that is in need of therapy or suspected of needing therapy. The subject can be a patient. The subject may have or be suspected of having a disease.

The term “patient” or “subject in need thereof”, as used herein, generally refers to a person who is receiving or is expected to receive treatment. For example, a patient can be a person who has been prescribed a dosage regimen of a drug formulation comprising ketamine.

The term “user,” as used herein, generally refers to a person who uses or operates a system, device, or application described herein. The user can be a doctor or medical practitioner who configures the drug delivery device or dosage regimen(s). In some embodiments, the user is an authorized user who provides authentication information (e.g., authorization code or biometrics) to unlock the device or otherwise gain access to the dosage regimen settings. The user can be a subject who uses the drug delivery device to administer a dose according to the dosage regimen. The subject who self-administers doses of the drug formulation is generally not able to configure the dosage regimen.

The term “tautomer,” as used herein, refers to one of two or more structural isomers which exist in equilibrium and which are readily converted from one isomeric form to another. It will be apparent to one skilled in the art that certain compounds, such as NMDA receptor antagonists like ketamine, may exist in tautomeric forms, all such tautomeric forms of the compounds being within the scope of the present disclosure.

The NMDA receptor antagonist of the present disclosure, such as ketamine, may also contain unnatural proportions of atomic isotopes at one or more of the atoms that constitute such compounds structures. For example, the NMDA receptor antagonist may be radiolabeled with radioactive isotopes, such as for example tritium (³H), iodine-125 (¹²⁵I) or carbon-14 (¹⁴C). All isotopic variations of the NMDA receptor antagonists of the present disclosure, whether radioactive or not, are encompassed within the scope of the present disclosure.

The term “substantially pure,” as used herein, generally refers to a purity of at least 90% or higher. In some embodiments, a substantially pure substance has a purity of at least 90%, 91%, 92%, 93%, 94%, 95%, 96%, 97%, 98%, 99%, or more.

The term “tamper resistant,” as used herein, generally refers to having one or more features designed to mitigate the risk of tampering or interfering with the normal functioning of a system, device, or method described herein.

A “therapeutically effective amount” or “effective amount,” as used herein, generally refers to the amount of a pharmaceutical agent required to achieve a pharmacological effect. The term “therapeutically effective amount” includes, for example, a prophylactically effective amount. An “effective amount” of an NMDA receptor antagonist, such as ketamine, is an amount effective to achieve a desired pharmacologic effect or therapeutic improvement. The effective amount of an NMDA receptor antagonist, such as ketamine, will be selected by those skilled in the art depending on the particular patient and the disease level. It is understood that “an effective amount” or “a therapeutically effective amount” can vary from subject to subject, due to variation in metabolism of an NMDA receptor antagonist, age, weight, general condition of the subject, the condition being treated, the severity of the condition being treated, tolerance of side effects, and the judgment of the prescribing physician.

“Treat” or “treatment” as used in the context of a physical, neurological and/or psychiatric disorder refers to any treatment of a disorder or disease related to the symptoms of the physical, neurological and/or psychiatric disorder, such as stopping or reducing the symptoms of the disease.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents. In embodiments, compounds of the present disclosure contain both basic and acidic functionalities that allow the compounds to be converted into either base or acid addition salts. The neutral forms of the compounds may be regenerated by contacting the salt with a base or acid and isolating the parent compound in a conventional manner. The parent form of the compounds differs from the various salt forms in certain physical properties, such as solubility in polar solvents, but, unless specifically indicated, the salts disclosed herein are equivalent to the parent form of the compound for the purposes of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain NMDA receptor antagonists of the present disclosure can exist in unsolvated forms as well as solvated forms, including hydrated forms. In general, the solvated forms are equivalent to unsolvated forms and are encompassed within the scope of the present disclosure.

“Pharmaceutically acceptable excipient” and “pharmaceutically acceptable carrier” refer to a substance that aids the administration of a compound, such as a NMDA receptor antagonist like ketamine, to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the present disclosure. One of skill in the art will recognize that other pharmaceutical excipients are useful in the present disclosure.

An “effective amount” is an amount sufficient for a compound to accomplish a stated purpose relative to the absence of the NMDA receptor antagonist (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). The therapeutically effective amount can be ascertained by measuring relevant physiological effects, and it can be adjusted in connection with the dosing regimen and diagnostic analysis of the subject's condition, and the like. By way of example, measurement of the serum level of a NMDA receptor antagonist such as ketamine or a hydrate, solvate, or pharmaceutically acceptable salt thereof (or, e.g., a metabolite thereof) at a particular time post-administration may be indicative of whether a therapeutically effective amount has been administered.

For any NMDA receptor antagonist described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan. Adjusting the dose to achieve maximal therapeutic window efficacy or toxicity in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to effect a beneficial therapeutic response in the patient over time. The size of the dose also will be determined by the existence, nature, and extent of any adverse side effects. Determination of the proper dosage for a particular situation is within the skill of the practitioner. Generally, treatment is initiated with smaller dosages that are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means intravenous, parenteral, intraperitoneal, intramuscular, or subcutaneous administration, or the implantation of a slow-release device, e.g., a mini-osmotic pump, to a subject. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, etc. By “co-administer” it is meant that a composition described herein is administered at the same time, just prior to, or just after the administration of one or more additional therapies (e.g., a benzodiazepine, a selective serotonin 5-HT3 receptor antagonist, a beta-blocker, and/or an inhibitor of CYP2B6 and/or CYP3A and/or CYP2C9). The compound (e.g., drug or active ingredient such as ketamine) of the present disclosure can be administered alone or can be co-administered to the patient. Co-administration is meant to include simultaneous or sequential administration of the compound individually or in combination (more than one compound or agent). Thus, the compositions can also be combined, when desired, with other active substances (e.g., to reduce metabolic degradation). The compositions of the present disclosure may additionally include components to provide sustained release and/or comfort. Such components include high molecular weight, anionic mucomimetic polymers, gelling polysaccharides and finely divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

By “co-administer” it is meant that a composition described herein is administered at the same time, just prior to, or just after the administration of one or more additional therapies. The compounds of the present disclosure can be administered alone or can be co-administered to the patient. Co-administration is meant to include simultaneous or sequential administration of the compounds individually or in combination (more than one compound).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to affect a beneficial therapeutic response in the patient over time. The size of the dose also will be determined by the existence, nature, and extent of any adverse side effects. Determination of the proper dosage for a particular situation is within the skill of the practitioner. Generally, treatment is initiated with smaller dosages that are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating drug dependence, psychiatric or neurological disorder, or pain disorders.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The phrase “in a sufficient amount to effect a change” means that there is a detectable difference between a level of an indicator measured before (e.g., a baseline level) and after administration of a particular therapy. Indicators include any objective parameter (e.g., serum concentration) or subjective parameter (e.g., a subject's feeling of well-being).

### Drug Delivery Device

In some aspects, disclosed herein are systems, devices, and methods for administering a drug formulation according to one or more programmed dosage regimen. In an exemplary embodiment, the drug formulation comprises ketamine. In some embodiments, the system or device comprises a pump for administering the drug formulation.

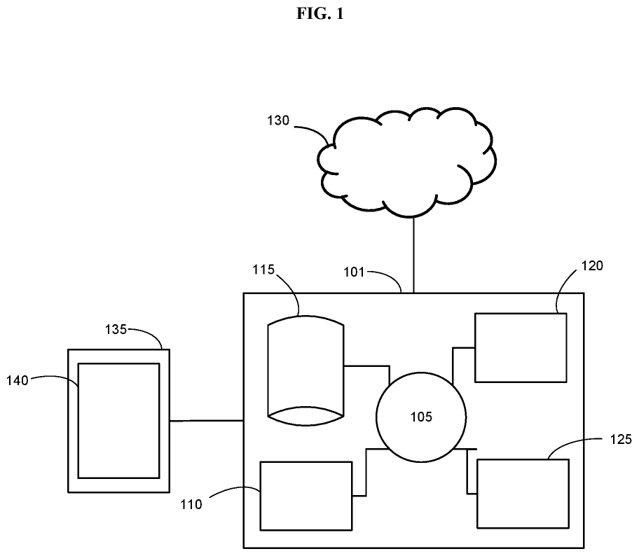

In some embodiments, a drug delivery device comprises a computer or computer system 101 as shown in FIG. 1. In some embodiments, the computer system comprises at least one processor 105 configured to carry out executable instructions to create a software application comprising one or more software modules 125 and configured for administering a dose of a drug formulation according to a programmed dosage regimen. In some embodiments, the drug delivery device comprises a memory 110, an electronic storage unit 115 (e.g., hard drive), a network adaptor or element for wired and/or wireless communications 120 with a network and/or cloud 130. In some embodiments, the application comprises a control module for configuring at least one dosage regimen according to instructions provided by a user. In some embodiments, the control module operates a pump mechanism to deliver a dose according to at least one programmed dosage regimen. In some embodiments, the control module operates the pump mechanism to deliver a dose selected by a user. In some embodiments, the control module limits or restricts the dose based on one or more dose limits. In some embodiments, a dose limit is set by an authorized or administrative user (e.g., a doctor or medical practitioner). In some embodiments, an authorized user is recognized based on entry of an authentication code or other authenticating information (e.g., biometrics).

In some embodiments, the drug delivery device has an authentication code (e.g., a password) whose entry allows configuration of the dosage regimen and/or dosage limit(s). In some embodiments, the drug delivery device logs user activity relating to changes in at least one dosage regimen and/or dosage limit(s) such as changes made and/or time of change. In some embodiments, the drug delivery device logs every instance the authentication code was entered such as the time and/or place. In some embodiments, the logged information is uploaded over a network to a remote server for storage. In some embodiments, the remote server is accessible by the authorized or administrative user to view and/or download the logged information. In some embodiments, a drug delivery device comprises a software application comprising a monitoring module allowing an authorized user (e.g., a physician for the subject) to remotely monitor at least one dosage regimen over a network. In some embodiments, the monitoring module provides usage data to a remote server that is accessible by the authorized user. In some embodiments, the monitoring module transmits usage data directly to a communication device of the authorized user (e.g., without using an intermediary or remote server). In some embodiments, the software application comprises a remote access module allowing an authorized user to remotely configure or modify at least one dosage regimen over a network. In some embodiments, the remote access module allows an authorized user to login and configure/re-configure the drug delivery device remotely such as over a network. For example, a subject may call his physician asking for a change to the dosage regimen, and the physician may remotely configure the new dosage regimen. In some embodiments, the remote access module allows an authorized user to unlock the drug delivery device remotely such as over a network. In some embodiments, the remote access module communicates with the authorized user over a Wi-Fi, Bluetooth, cellular connection, or a combination thereof.

In some embodiments, the drug delivery device comprises an unlocked mode during which the at least one dosage regimen can be configured and/or modified (e.g., by an authorized user). In some embodiments, the drug delivery device comprises a locked mode during which the at least one dosage regimen cannot be configured and/or modified (e.g., when the device is being used by a subject who is not an authorized user to self-administer and/or alter a dose). In some embodiments, the drug delivery device requires input of an authentication code such as one provided by a doctor or other healthcare provider in order to switch between a locked mode and an unlocked mode. In some embodiments, the drug delivery device switches from an unlocked mode to a locked mode after receiving user input to switch to the locked mode. In some embodiments, the drug delivery device switches from an unlocked mode to a locked mode after receiving user input to switch to the locked mode and input of an authentication code.

Alternatively, in some embodiments, the drug delivery device is locked by the manufacturer after being configured with at least one pre-programmed dosage regimen. In some embodiments, the drug delivery device cannot be unlocked after being locked by the manufacturer such that even a healthcare provider for the subject is unable to reconfigure the at least one dosage regimen (e.g., device is permanently locked). In some embodiments, this permanent lock prevents abuse of the drug delivery device whereby the subject gains access to an authentication code of the healthcare provider by foreclosing the possibility of anyone being able to change the dosage regimen.

In some embodiments, the drug delivery device comprises a pump mechanism configured for administering the drug formulation. In some embodiments, the pump mechanism is configured for pumping a fluid such as a fluid drug formulation (e.g., ketamine HCl). In some embodiments, the pump mechanism is configured to couple with a reservoir for storing the drug formulation. In some embodiments, the pump mechanism is configured to detachably couple with a cartridge for storing the drug formulation. In some embodiments, the cartridge is reusable. In some embodiments, the cartridge is disposable. In some embodiments, the cartridge is a single-use disposable cartridge. In some embodiments, the cartridge is configured to be tamper resistant.

In some embodiments, the drug delivery device comprises a user interface 135 allowing a subject to self-administer a dose of the drug formulation according to at least one programmed dosage regimen. In some embodiments, the user interface comprises a display screen 140. In some embodiments, the user interface comprises at least one interactive element for receiving user input. In some embodiments, an interactive element is a physical interactive element such as, for example, physical buttons, knobs, dials, switches, toggles, wheels, click wheels, keyboard, or any combination thereof. In some embodiments, a user interacts with an interactive element by touching, tapping, swiping, twisting, turning, clicking, or pressing the element. In some embodiments, a user interface comprises one or more physical interactive elements (e.g., hard buttons). In some embodiments, a physical interactive element is a power button, a volume toggle button, a home button, a back button, menu button, navigation button(s), return button, multi-tasking button, camera button, a button on a physical keyboard, or any other physical button on the device. In some embodiments, the user interface comprises a display screen showing information about the dosage regimen and/or the current dose. In some embodiments, the display screen is an interactive touchscreen. In some embodiments, the user interface comprises a display screen showing information about the dosage regimen and/or the current dose. In some embodiments, a user interacts with the display screen using one or more physical interactive elements. In some embodiments, a user interacts with the display screen using one or more non-physical interactive elements (e.g., soft buttons on a touchscreen). In some embodiments, the user interface presents a user with one or more command options. In some embodiments, the one or more command options include at least one of administering a bolus of the drug formulation, commencing a continuous infusion of the drug formulation, pausing or cancelling a dose, accessing an activity log (e.g., record of doses administered), accessing a dosage regimen (e.g., for review or for configuration depending on user authorization), and accessing device settings.

In some embodiments, the drug delivery device comprises at least one network element for carrying out wireless communications. In some embodiments, the at least one network element comprises a radio transceiver for communicating wirelessly over radio waves. In some embodiments, the at least one network element comprises a Bluetooth transceiver for communicating with one or more Bluetooth-enabled devices (e.g., a smartphone, a Bluetooth beacon, etc.). In some embodiments, the at least one network element comprises a WiFi transceiver for communicating with one or more WiFi-enabled devices (e.g., a WiFi router, a smartphone). In some embodiments, a network element communicates over a network using short-range communications with network or communication devices in close proximity (e.g., a personal area network). Examples of technologies that utilize short-range network communications include wireless headsets or earbuds and wireless wearable sensors (e.g., Fitbit). Short-range wireless technologies include communications standards such as ANT, UWB, Bluetooth, ZigBee, and wireless USB. In some embodiments, a drug delivery device uses short-range wireless technologies to communicate with a nearby device such as a subject's smartphone, which then optionally communicates or relays the communications to a remote authorized user. In some embodiments, the drug delivery device communicates using Wi-Fi and/or a cellular network (e.g., 2G, 3G, or 4G networks) to send and receive communications. In some embodiments, the drug delivery device establishes a communication channel with a communication device such as by “pairing” with the device. In some embodiments, the drug delivery device establishes an ongoing or temporary communication session with a communication device. In some embodiments, the communication session comprises data transfer between the drug delivery device and the communication device.

In some embodiments, the communication device comprises a processor that executes instructions to create a software application allowing monitoring and/or uploading of data from the drug delivery device. In some embodiments, the software application comprises a data module storing usage data for the device. In some embodiments, the data module stores information for doses administered by the subject. In some embodiments, the data comprises information on access times such as when the device has been accessed, who accessed the device (e.g., authorized or unauthorized user, subject or healthcare provider), doses administered (time, administered amount, administration rate, duration of administration, dosage number according to the dosage regimen, etc), user information (e.g., name, age, address, etc). In some embodiments, the data is stored on the drug delivery device. In some embodiments, the data is transmitted to the communication device. In some embodiments, the data is sent to a remote server. In some embodiments, the data is provided to the authorized user and/or healthcare provider for the subject. In some embodiments, the data is encrypted. In some embodiments, the data is sent via encrypted data channel(s). In some embodiments, the data is subject to 128 bit or 256-bit encryption. In some embodiments, the data is sent as encrypted files over one or more encrypted channels. In some embodiments, the remote server is part of a HIPAA compliant data center. In some embodiments, the remote server is HIPAA compliant. In some embodiments, the drug delivery device data storage (e.g., hard drive) has file/folder encryption, full disk encryption, or both. In some embodiments, data encryption is carried out according to the Advanced Encryption Standard (AES) for encryption.

In some embodiments, the application comprises a communication module configured to communicate wirelessly with a remote authorized user (e.g., using a network element). In some embodiments, the communication module allows messages or requests to be sent by the user of the drug delivery device to the remote authorized user (e.g., requesting a change to the dosage regimen and/or dosage limit). In some embodiments, the communication module is configured to receive instructions configuring or modifying at least one dosage regimen and/or dosage limit from the remote authorized user. In some embodiments, communications are provided to a remote authorized user indirectly by transmission to a server or communication device accessible by the remote authorized user. In some embodiments, the communication device is a computer, tablet, or phone accessible by the remote authorized user. In some embodiments, the server makes the communications available to the remote authorized user via a web application programming interface (API) that can be accessed by an Internet-enabled electronic device. In some embodiments, communications are provided to a remote authorized user via SMS (short message service), MMS (multimedia messaging service), email, or a chat application (e.g., Google chat, instant messenger, etc.).

Some embodiments of the present disclosure relate to various ways to create a reusable, delivery device. In some embodiments, the delivery device is waterproof. Past solutions range from throw away devices to very expensive and large pump systems. The mechanical sealing of a system has been difficult with removable power systems and cords and communications. The media storage and delivery is also a key problem in past systems and control and authentication thereof. Accordingly, the present disclosure enables simple, reliable solutions that provide a more positive outcome. For example, past wearable solutions are not designed for waterproof use and typically are not designed for everyday use. In addition, other delivery devices on the market actually enclose all of the components and require the user to dispose of the system, which increases overall cost. Size and portability has also been limited. Therefore, embodiments of the present disclosure include a cartridge system allowing better cleaning and ease of use.

In some aspects, disclosed herein are methods of sealing the system, device, and/or cartridge. In some embodiments, provided herein is an ultrasonically sealed enclosure that creates a completely sealed device. In some embodiments, a vent (e.g., GoreTex vent) is provided to allow flexing within pressurized altitudes and temperature changes while preventing moisture from entering. For instance, exposure of a delivery device to hot outdoor environments and cold environments can create enormous pressures that the vent could protect against while limiting moisture from entering maintaining the structure and waterproof solution.

In some embodiments, disclosed herein are systems, devices, and methods for monitoring and providing feedback of safety parameters and patient pain rankings. This addresses a problem with securing the delivery material and the device. In some embodiments, the physician provides a prescription, for which the dosage/treatment regimen is monitored including recording the method and/or measurement parameters. Thus, the present disclosure provides methods to track and learn from each user for a prescription and optionally ranks the propensity for patient reactions and functionality (e.g., responsiveness, efficacy of treatment in reducing pain) to a given regiment.

In some aspects, disclosed herein are drug delivery devices. In some embodiments, these drug delivery devices are wearable devices configured to be worn or attached to the body, garment, or other worn equipment of a user (e.g., clipped to a belt, worn on a wrist band, etc.). Embodiments of these wearable devices provide several key solutions to past problems that have been observed and modified for better results in the wearable environment. For example, one challenge in the wearable device space is that the patient is expected to wear a device with and function in life normally. In some embodiments, the wearable device is configured to understand its environment and/or usage to enhance performance and/or understand its own function. For example, being in water or in wet environments and understanding when this is happening is important. In some embodiments, the wearable device comprises conductive and capacitive electrodes configured to monitor the relationship to the body, for example, in which the conductive portion monitors skin resistance. In some embodiments, one or more sensors allow decisions to be made based on sensor data. In some embodiments, sensor data is analyzed to determine the presence of a wet environment. In some embodiments, the wet environment is a wet environment external to a user or subject using the wearable device. In some embodiments, sensor data is analyzed to determine the presence of sweat or perspiration. In some embodiments, the sensor data comprises position as it relates to the body such as, for example, position/location of the sweat or perspiration. In some embodiments, the user is verified by the measurement of impedance between the two electrodes thus verifying to the delivery device control that the patient (e.g., the user) is present and the system is connected when that signal is connected and combines with the capacitive sensor detecting the body mass. In some embodiments, the user is verified with a mobile device ID, a cartridge ID, and their registration to the patient and/or physician. In some embodiments, this unit sends an ID code to the mobile device. In some embodiments, the mobile device is connected to a database such as a database stored on the cloud. In some embodiments, the mobile device is connected to the internet. In some embodiments, the connection utilizes an RF signal. In some embodiments, the link between the mobile device and the wearable device is BTLE, and a cellular link connects the mobile device to the database via the internet. Alternatively, the RF signal is a proprietary server frequency for additional security with a proprietary hub retained within the patient household. In some embodiments, data such as user statistics, processing pain, safety statistics, or any combination thereof are retained and measured over time. In some embodiments, the user charges the delivery device wirelessly until the device is fully charged by placing it on a charging device. In some embodiments, the user pairs and authenticates the mobile device and mobile application via database and/or network authentication. In some embodiments, the user inserts the cartridge and the system verifies and authenticates if the cartridge is valid or if it has been tampered with. In some embodiments, the wearable delivery device and system is authorized using first the database registrations for the cartridge ID, patient ID and password, patient mobile device Mac address ID, the delivery device ID, various device and cartridge security challenges (e.g., security challenge questions), present level and usage data, or any combination thereof. Once authenticated, in some embodiments, the database provides the prescribed delivery options, timing and delivery options. In some embodiments, the device is prepared for skin placement and optionally begins by running a portion of the delivery material. In some embodiments, the cannula moves past the septum and delivers a small amount of fluid. In some embodiments, the position of the cannula after moving past the septum indicates a valid cartridge and/or tampering or past usage, which are optionally stored on the RFID tag as dosage is delivered and past positions are logged on the cartridge. In some embodiments, the device is positioned or attached on the skin with adhesive, straps, elastic bands or other viable mechanical means. In some embodiments, one or more sensors detect the body and optionally set a body contact flag. In some embodiments, the mobile device (e.g., smartphone) authorizes and/or enables automatic cannulas insertion. In some embodiments, the patient presses a button on the mobile software application to cause instructions to be sent to the gear drive of the delivery device to insert the cannulas. In some embodiments, the cannula insertion is verified by using a tiny magnet that moves with the cannulas insertion body in which a Hall Effect semiconductor indicates a proper insertion position. In some embodiments, the protocol (e.g., a dose of a dosage regimen) is then run for that user unless the cartridge or device is removed. In some embodiments, the device delivers the full volume of the cartridge over the prescribed time, or some fraction of its full capacity as prescribed by the physician according to the dosage regimen. In some embodiments, the device allows a user to request additional dosage as it tracks pain level (e.g., user provides feedback on pain level during treatment). In some embodiments, the device allows a user to request additional dosing in either bolus or an increased basal rate (e.g., increasing the infusion rate of the drug). In some embodiments, the device allows a variety of protocols to be entered and administered. In some embodiments, the drug composition or formulation can be formulated to the desired effect based on dosages and delivery volumes of the device. In some embodiments, the pump is a gear drive screw drive. In some embodiments, the pump comprises a sensor configured to track plunger position. In some embodiments, the screw drive comprises a threaded rack molded in plastic that can be flexed about the inner package to accommodate smaller spaces and guided with plastic molded details to form a half loop and utilize a gear drive. In some embodiments, the screw drive is a blade that has a gear drive on one side that can flex about its thin side to enable a flexible rack drive. In some embodiments, the device comprises factor settings that are calibrated to retracted, started, pushed, completed volumes, or any combination thereof. In some embodiments, the device comprises a controller configured to monitor the one or more sensors for body contact, time using a real time clock, status of the cartridge, or any combination thereof. In some embodiments, the device comprises at least one accumulator configured to accumulate dose for the cartridge first locally and optionally then stores the usage on the cartridge. In some embodiments, the at least one accumulator store the usage on the cartridge via RFID after every dose so the cartridge has usage data to confirm dosing and authenticated usage. In some embodiments, the cartridge stores use by dates, patient ID, device ID, mobile ID, dose start date, removal flag, pain scales, or any combination thereof as authenticators for preventing tampering and reuse. In some embodiments, the device comprises tramper resistance features related to sealing the drug reservoir within the pump in such a manner that it is difficult to access the medication contained within the reservoir either, after it is coupled to the pump by the user, or after it is coupled to the pump by the manufacturer, pharmacy, clinician or other certified person. In some embodiments, the reservoir has a sliding window that is moved to cover the medication fill port after the reservoir is loaded. In some embodiments, the reservoir rotates to hide the fill port internally. In some embodiments, the reservoir is completely sealed inside the device after it is filled at the factory, the pharmacy, the clinician office or other certified location.

Waterproof System with Removable Cartridge

In some embodiments, the systems, devices, and methods disclosed herein provide a delivery device with a removable cartridge and cannula(s). In some embodiments, the delivery device is sealed, reusable, waterproof, or any combination thereof. In some embodiments, the cartridge when inserted aligns the plunger, the cannulas drive, the RFID reader and the magnetic sensors to enable an intrinsic relationship maintaining an easy to use device. In some embodiments, the device is configured with waterproof design. In some embodiments, the device utilizes a rechargeable battery and comprises a power management system configured to charge the battery utilizing a wireless power system.

Cartridge Detection System

In some embodiments, the systems, devices, and methods disclosed herein provide a cartridge detection system. In some embodiments, the cartridge system utilizes a power harvesting near field communications system. In some embodiments, the harvested power is used to power an LED and sensor to enable level detection. In some embodiments, the system comprises a thin film resister with a wiper attached to the plunger to provide a resistance that is converted to a voltage to indicate plunger position. In some embodiments, the plunger uses a magnet and a Hall Effect device to show position. In some embodiments, the system comprises an RFID tag configured to provide data and sensor feedback and/or a unique pre-programmed code for cartridge security.

Body Detection

In some embodiments, the systems, devices, and methods disclosed herein provide a body detection system using capacitive and/or resistive sensors to enable and track body contact. In some embodiments, the resistive sensors use simple spring loaded contacts that are pressed against the skin. In some embodiments, the sensors detect general skin resistance and water contact events for data analysis. In some embodiments, the capacitive sensors are used to detect proximity to the body and obtain significant sensor reading changes when that proximity changes.

Tamper Circuit and Sensors

In some embodiments, the systems, devices, and methods disclosed herein provide a cartridge comprising an RFID circuit that includes traces printed over the cartridge that when broken disable the device and indicate improper use through the data interface or cloud interface. In some embodiments, additional authentication is executed when doses are compared with plunger position over time. In some embodiments, the system generates an error when the user violates these parameters showing improper use or tampering.

Multi-Layer Security System

In some embodiments, the systems, devices, and methods disclosed herein provide a multi-tier security system requiring the cartridge, the mobile device, the delivery device, or any combination thereof to report a security challenge response for each unique number relating to the cartridge, the device, the mobile device, or any combination thereof. In some embodiments, the registered devices and cartridges for a specific user are part of the security challenge. As part of the pairing, these numbers for that user and for these devices must be authenticated for the enable code. If security is breached the device and cartridge can be disabled from further use. This disable code and error cause is sent to the network for registration.

Heart Rate Sensor

In some embodiments, the systems, devices, and methods disclosed herein provide a heart rate sensor configured to track heart rate over time and optionally proximity to the user for data related to the delivery system.

Cannulas Insertion with Magnetic Position Sensor

In some embodiments, the systems, devices, and methods disclosed herein provide a cannulas system. In some embodiments, the cannula system comprises a magnetic element that can be monitored with a Hall Effect sensor. This enables a low drag simple system for determining cannulas position. In some embodiments, the magnetic element and sensor allow the spring loaded or the motor driven cannulas system to indicate insertion easily.

Local Pain Button

In some embodiments, the systems, devices, and methods disclosed herein provide one or more buttons. In some embodiments, when a user taps a second capacitive button one to five times (or preset pattern), the system logs locally an acute pain event and shares that with the pain-tracking log. In some embodiments, pressing the button drives a dose directly if that option is available by prescription. In some embodiments, the device shares that information and log with the physician, e.g., over the cloud or network.

Pain Tracking

In some embodiments, the systems, devices, and methods disclosed herein provide an app for tracking pain medication delivery. In some embodiments, the system tracks pain medication delivery through the device firmware and/or an app using the paired mobile device and data taken from the delivery device. In some embodiments, the delivery can be driven to a maximum medication dosage or maximum delivery of medication within a specified period authorized by the physician within a period of time through the ability for user requests for pain medication on demand. In some embodiments, as these pain reduction requests are made and at given intervals, the system or device enables push notifications that request pain ratings from the patient. This information can be used to drive delivery and feed information back to the prescribing doctor.

Factory Sealed Drug Reservoir

In some embodiments, the systems, devices, and methods disclosed herein provide a drug reservoir. In some embodiments, the drug reservoir stores a such as ketamine. In some embodiments, the drug reservoir stores a drug that is a controlled substance. In some embodiments, the drug reservoir stores a drug that is susceptible to abuse. In certain embodiments, a proprietary formulation (e.g., containing ketamine) is loaded either in the factory or in the pharmacy, after which the fill port is be sealed off through any of variety of mechanisms including: a locking window, locking outer shell, rotation of the fill port away from the fill window, an outer shell sealed after drug reservoir is inserted, or other mechanical design.

Pre-filled Reservoirs: In certain embodiments, a proprietary ketamine formulation is provided to the patient in prefilled reservoirs.

Pain Scales

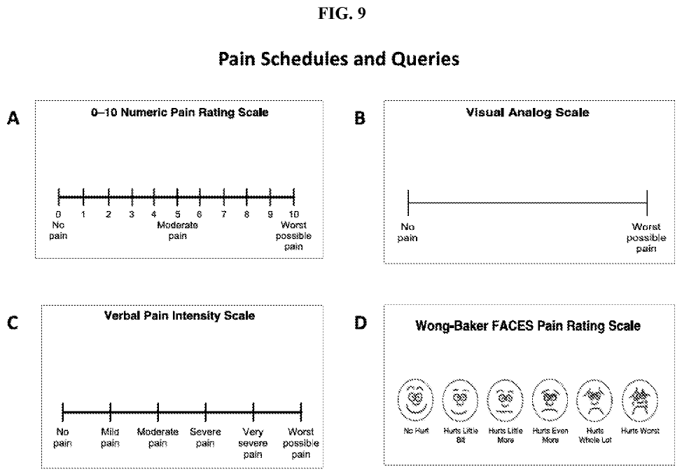

In some embodiments, the systems, devices, and methods disclosed herein allow user entry of pain scale information or data. In some embodiments, the device prompts the user to enter pain scale data in an accepted mode at specific times. Prompts can be given for data entry as per the request of the supervising doctor and/or as per various programs for assessment of pain management efficacy. In some embodiments, the drug delivery device (e.g., ketamine pump for subcutaneous administration) comprises an interface allowing a user to enter pain scale information such as, for example, entering a number indicative of a level of pain. In some embodiments, the communication device or mobile device of a user (e.g., a smartphone) comprises a software application (e.g., a mobile app configured to communicate with the drug delivery device) allowing a user to provide pain scale information. As an example, patients are asked to enter data before an acute bolus of ketamine is given and at one and two hours after delivery. This data can be provided to a supervising physician (e.g., over a network such as the internet) who can use this data to determine, modify, or optimize an effective treatment plan and dosing range (e.g., adjust the dosage regimen remotely or on-site when the patient brings in the drug delivery device). Examples of potential pain scales include but are not limited to: Numeric Pain Rating Scale, Visual Analog Scale, Verbal Pain Intensity Scale and Wong-Baker FACES Pain Rating Scale. Examples of these scales are shown in FIGS. 9A-9D.

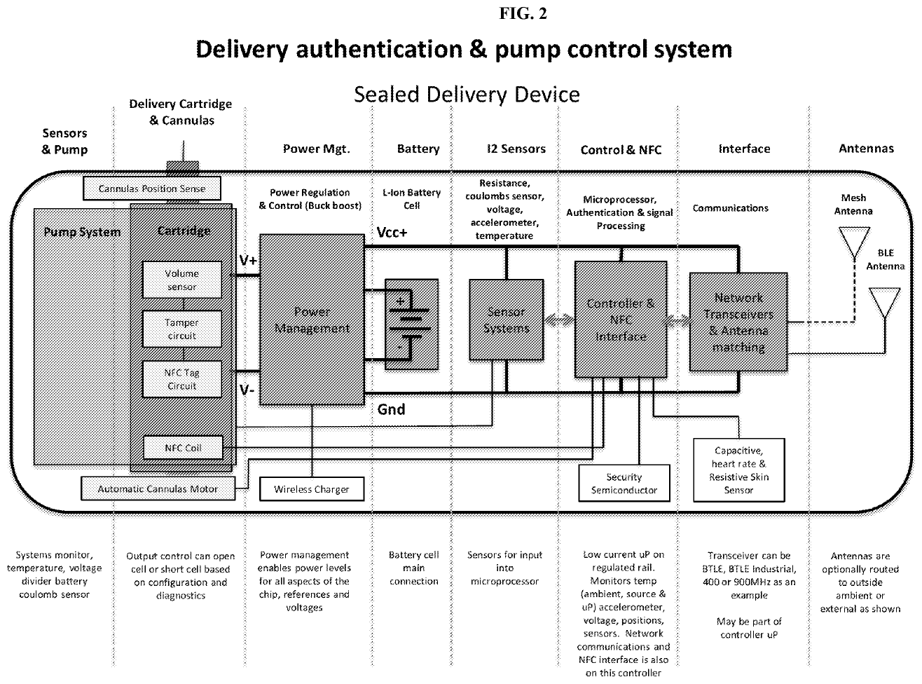

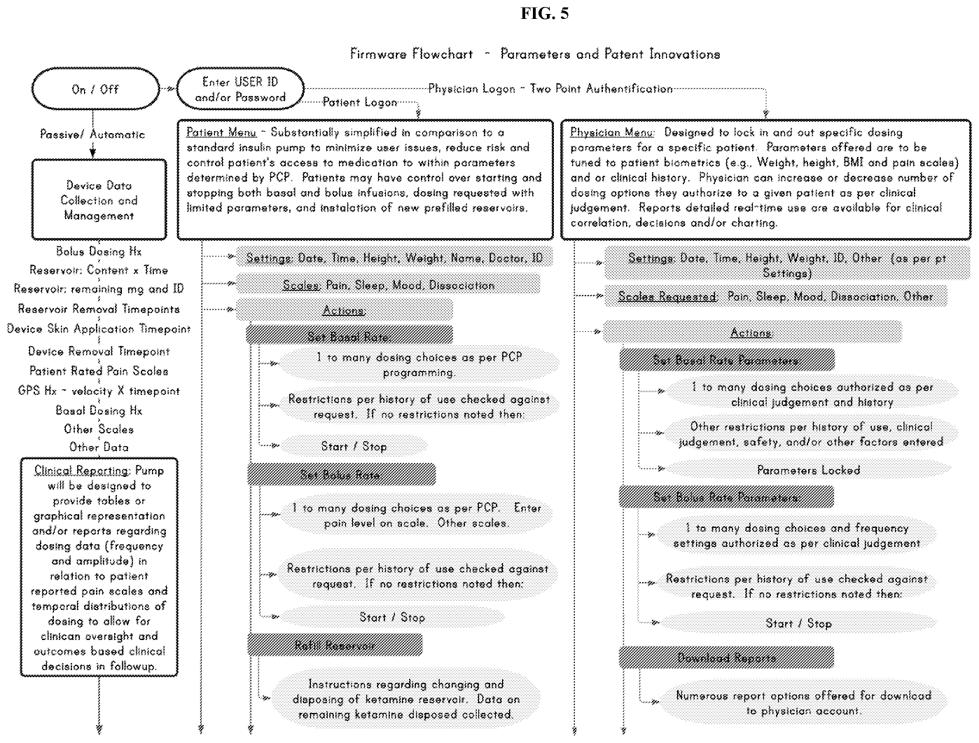

FIG. 2 illustrates one embodiment of a delivery system that is designed to be modular and waterproof. The system utilizes a cartridge that has a NFC powered tag and monitoring system. An example of such an energy harvesting system has been developed by NXP Semiconductors in the OM5569/NT322E development kit. This technology allows the design to make the cartridge smarter and powered by the delivery device. The delivery device stores information in parallel to the cartridge as an additional authentication measure. When authenticated doses are delivered both accumulators are updated locally and on the cartridge tag securely. The delivery device contains a rechargeable battery and the power management system maintains the power and charging for additional safety. Only the proper power can be delivered to the battery by our profile maintaining battery safety from thermal events. The power management system has a protection circuit that monitors the battery temperature and removes it from circuit if any anomalies occur. A sensor reads the cannulas position and enables opportunities to push materials for clearing the cannulas as well as cannulas deliver and insertion as well as retraction below the septum material. The whole delivery tube, hose and cannulas are part of the removable cartridge system. When placed in the delivery system the tag is read, the unique ID allows a security challenge and pairs the registration data for secure authentication. The gears for the cannulas mechanism and match up upon insertion. The plunger is located to mate up with the cartridge and is a gear driven screw drive that has a position sensor. It knows by factory settings where the unload/load positions and where the cartridge empty position may be. The cartridge empty position may also be overridden by the tag data depending on dosages and delivery/volume mechanisms. Unloading by the press of a button retracts the cannulas and removes the plunger but the cartridge volume and actual plunger remains at the last know position. This information is used to identify volumes unused (e.g., unused formulation remaining in the cartridge) or reuse authentication (e.g., allow reuse of unused formulation) or disabling (e.g., disallowing continued use of the unused formulation if it exceeds a preset or preprogrammed dosage regimen). The tamper circuits in the cartridge can be simple printed electronics on a label that when removed or broken a circuit is enabled to show tampering and invalidates the cartridge. The tamper proof capability is enabled in a label and utilizes the RFID as a means to read the protection circuits. The label is designed with perforations so that if removed or portions are removed the circuit is broken and the tamper provision is triggered relating a signal or flag to the device controller and to the cloud and mobile device. The function of the plunger breaks the main seal and also verifies proper use and tamper resistance alone with plunger location, etc. The information in the controller follows the chart in FIG. 5 and requires the physician ID, the Patient ID, the device ID the mobile device ID and the Cartridge ID to all authenticate before usage. They are pre-registered to that user and the mobile application requires a password and user ID for authentication on the network from both the user and the physician to enable the system. The communication is enabled via BTLE through the mobile cellular device but may also be a proprietary network for a higher level of security and local functionality. The home hub can assure use and validation only within that area of use and can limit zones of operation. That same can be done with a mobile phone and for example can disable the pump when traveling above a specific speed. Geo-tagging is another layer of security allowing the device to be enabled or authenticated only within a preset space or home. The mesh antenna is designed to allow the dual purpose of having the specific region of hub use and interface. The capacitive and resistance sensors indicate when the delivery device is wet, when the device is connected to a body and when it is not in proximity to a body. The capacitive sensor verifies that the presence of impedance is also present assuring body detection with no single point of failure. The semiconductor safety device enables a unique ID for IOT devices that assures proper firmware upgrades and secure communications making the device very difficult to hack. The NFC reader both powers the cartridge electronics and allows two-way data to be shared with the cartridge. Data stored on the cartridge includes manufacturing data, use by data, lot codes, dose time and amounts, dose accumulator for cartridge, over all time in device, time to empty, pain scale data and more. In some embodiments, the delivery device uses these basic accumulators but has a second set that maintains the last ten cartridges and accumulates overall dose for each specific material over time including total time of use, charges, time on body, times removed, proper cannulas insertions, pain dose requests locally (via pushbutton) accumulated and per cartridge and pain scale data, and more.

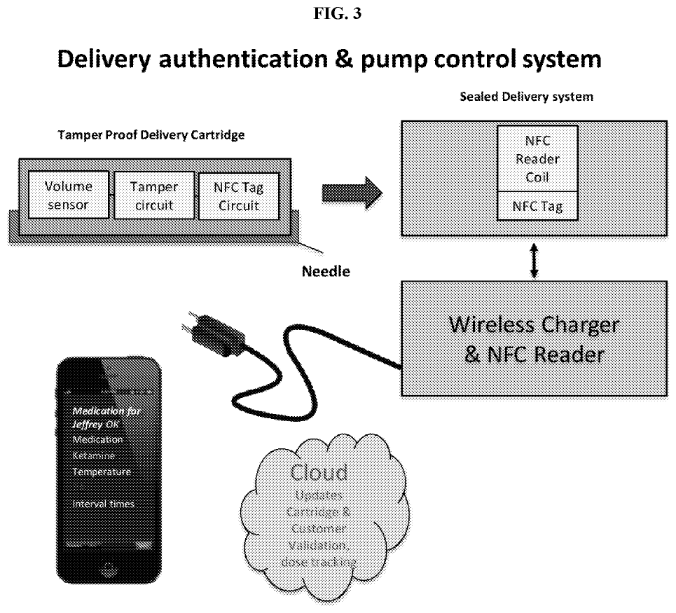

FIG. 3 shows an embodiment of the cartridge, the delivery device the mobile device, the charger base and the cloud or network interface for collecting and authenticating the required information to enable the system for use. FIG. 3 schematically illustrates a delivery authentication and pump control system with a tamper proof delivery cartridge and a sealed delivery system. The delivery authentication & pump control system shown includes a wireless power charging system, a removable cartridge with a power harvested sensor feedback system, and a waterproof delivery device.

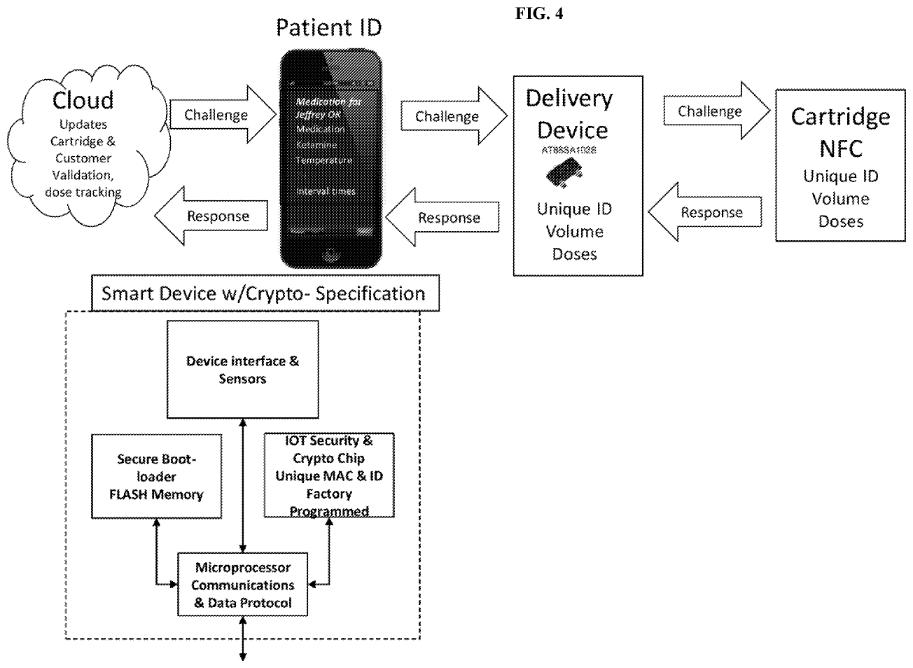

FIG. 4 shows an embodiment of the security challenges provided to assure authentication and proper alignment with the physician. FIG. 4 schematically illustrates a patient verification and authentication pathway for the drug delivery device disclosed herein. The multi-layer security system is shown using a phone ID, a device unique ID, a cartridge unique ID and a secure cloud or network database to provide a security challenge. The challenge responses are passed for a challenge response.

FIG. 5 shows an embodiment of the physician and user menus as determined by device firmware and device regulation designed to produce a dual capacity in device wherein only the physician can set medication delivery protocols and the patient can access those protocols to deliver medication as their clinical needs dictate within those protocols permitted. FIG. 5 schematically illustrates an embodiment of a two-point authentication pathway interfacing between the patient screen and the physician screen. The patient controlled menu is displayed on the left hand pathway. The physician order menus are displayed on the right hand pathway.

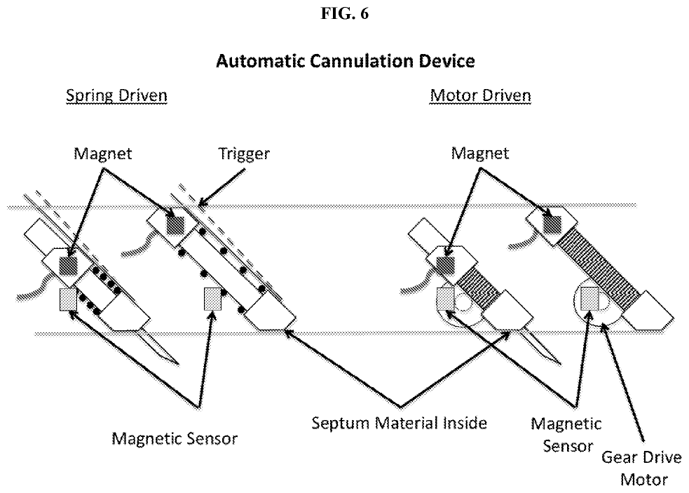

FIG. 6 shows the automatic cannulas insertion and position device with septum material. It shows a spring driven system and a gear driven system. The spring driven and gear driven automatic cannulas systems are triggered electromagnetically by the controller. FIG. 6 shows a diagram of an automatic cannulation device as a spring loaded and a gear driven cannulas system with a magnetic position sensor. The cannulas sensor may include a septum to protect the materials for transport.

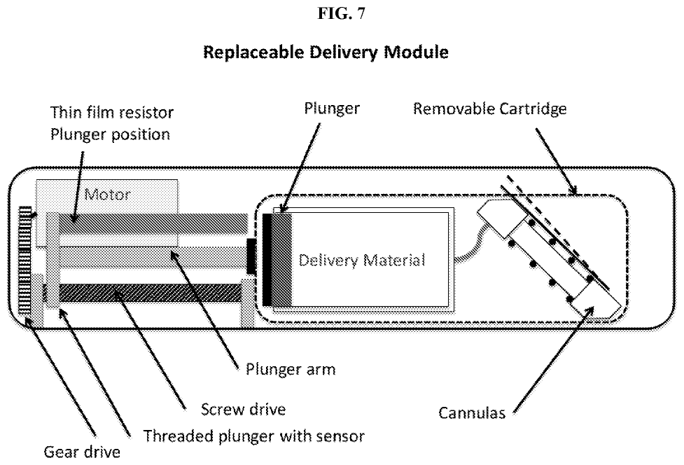

FIG. 7 shows the gear plunger drive that drives the syringe to deliver the material requires. It defined the cartridge and the sensor configurations for monitoring proper usage. FIG. 7 shows a diagram of a replaceable delivery module with a plunger drive and the removable cartridge of one embodiment of the delivery system disclosed herein. In some embodiments, the pump mechanism and the drug cartridge containing the drug formulation can be decoupled allowing insertion of a new drug cartridge for continuing treatment. In some embodiments, the cartridge is pre-filled, replaceable, and configured without an exposed fill to discourage the potential for tampering. In some embodiments, the pump mechanisms and/or housing may be replaced every one to three days. In some embodiments, the pump mechanisms and/or housing may be semi-durable, designed to be replaced every one to 6 months. In some embodiments, the pump mechanisms and/or housing is configured to be durable (e.g., not temporary or disposable), for example, designed to be replaced every 1 to 4 years.

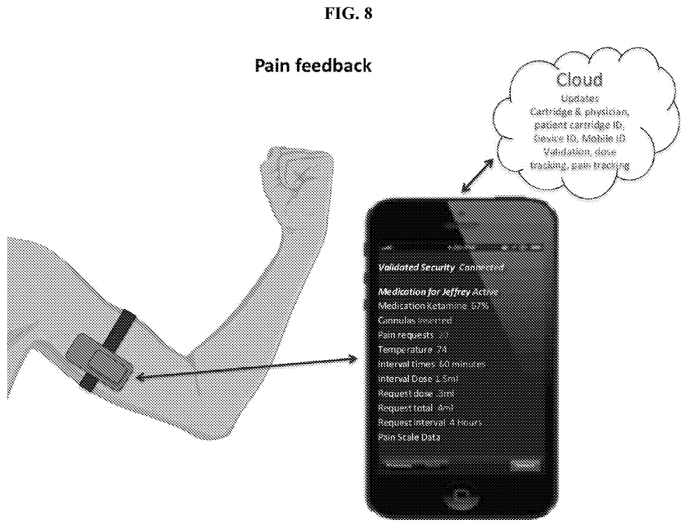

FIG. 8 shows an embodiment of a delivery device with the cartridge inserted communicating to an authorized mobile device and communicating data. The delivery device can be tapped by the user in a pattern to indicate pain. The device may vibrate or beep to confirm and an additional pattern of tap is required to confirm. Each request ID is logged for future reporting. FIG. 8 shows an illustration of the communication links between a wearable device, a user communication or mobile device, and the cloud in one embodiment of the present disclosure for providing a pain tracking system and feedback.

FIGS. 9A-9D show a number of potential pain tracking schedules and queries that may be employed to track pain data in real time use of the device by the patient for use in clinical reporting, charting and treatment planning.

Directional terms, such as “vertical,” “horizontal,” “top,” “bottom,” “upper,” “lower,” “inner,” “inwardly,” “outer” and “outwardly,” and “thinner” are used to assist in describing the present disclosure based on the orientation of the embodiments shown in the illustrations. The use of directional terms should not be interpreted to limit the invention to any specific orientation(s).

### Intramuscular or Subcutaneous Injection

Described herein are systems, devices, and methods for delivery of drug formulations such as by intramuscular or subcutaneous injection that is not limited to the hospital or clinic setting. Intramuscular or subcutaneous injection avoids certain drawbacks found in oral, sublingual, nasal, and rectal modes of administration. Intramuscular or subcutaneous injection allows higher drug absorption by avoiding first pass metabolism. In the case of ketamine, intramuscular or subcutaneous infusion allows a higher proportion of the total delivered drug to remain in the active, effective form of racemic and/or s-ketamine (e.g., in an untransformed state) rather than biotransformation through first pass metabolism into less effective metabolites including but not limited to: S-norketamine, R-norketamine, S-dehydronorketamine, R-dehydronorketamine, 2S,6R-hydroxyketamine, 2R,6S-hydroxyketamine, 2S,6S-hydroxyketamine, and 2R,2S-hydroyyketamine. Accordingly, total body exposure to ketamine and to ketamine metabolites in the course of treatment is reduced compared to the current art by allowing lower total dosing per treatment. This decreases the burden placed upon the body in detoxification, thus reducing associated risks such as bladder dysfunction. Moreover, removal of first pass metabolism improves interpatient dosing range reliability in treatment by reducing the effects of interpatient variation in CYP3A and/or CYP2B6 and/or CYP 2C9 enzymes known to cause large variations in plasma concentration in administration with first pass metabolism. This can also reduce some of the interpatient variability in plasma concentration levels due to concurrent use of CYP3A and/or CYP2B6 and/or CYP 2C9 inhibitors, substrates or inducers.

### Programmed Dosage Regimen

Described herein are programmed dosage regimens for use with the systems, devices, and methods of the instant disclosure. In some embodiments, a dosage regimen comprises a series of doses. In some embodiments, a dosage regimen comprises a plurality of dosing options selectable by the subject and/or user. For example, a dosage regimen comprises three selectable dose options: a single continuous infusion dose at 1 mg/hour, a single continuous infusion dose at 2 mg/hour, and a low bolus injection of 1 mg that repeats every hour. In some embodiments, a dosage regimen comprises at least 1, 2, 3, 4, 5, 6, 7, 8, 9, 10, 11, 12, 13, 14, 15, 16, 17, 18, 19, or 20 or more dosing options and/or no more than 1, 2, 3, 4, 5, 6, 7, 8, 9, 10, 11, 12, 13, 14, 15, 16, 17, 18, 19, or 20 or more dosing options. In some embodiments, a dosage regimen comprises one or more dosage limits. In some embodiments, a dosage regimen comprises dosage duration (e.g., time period to infuse a single dose). In some embodiments, a dosage regimen comprises treatment duration (e.g., time period of entire dosage or treatment regimen). In some embodiments, the dosage regimen is configured for administration of a drug formulation comprising ketamine (e.g., ketamine HCl). In some embodiments, a programmed dosage regimen comprises a continuous infusion dose. In some embodiments, a continuous infusion dose is optionally paused and continued according to user input. In some embodiments, a continuous infusion dose comprises an infusion rate of at least 0.01, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or at least 200 mg/hour and/or no more than 0, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or no more than 200 mg/hour of an active ingredient such as ketamine.

In some embodiments, a continuous infusion dose comprises an infusion rate of at least 0.0001, 0.001, 0.002, 0.003, 0.004, 0.005, 0.006, 0.007, 0.008, 0.009, 0.01, 0.015, 0.20, 0.025, 0.03, 0.035, 0.040, 0.045, 0.050, 0.055, 0.060, 0.065, 0.070, 0.075, 0.080, 0.085, 0.090, 0.095, 0.1, 0.11, 0.12, 0.13, 0.14, 0.15, 0.16, 0.17, 0.18, 0.19, or at least 0.2 milligrams/kg/hour or no more than 0.0001, 0.001, 0.002, 0.003, 0.004, 0.005, 0.006, 0.007, 0.008, 0.009, 0.01, 0.015, 0.20, 0.025, 0.03, 0.035, 0.040, 0.045, 0.050, 0.055, 0.060, 0.065, 0.070, 0.075, 0.080, 0.085, 0.090, 0.095, 0.1, 0.11, 0.12, 0.13, 0.14, 0.15, 0.16, 0.17, 0.18, 0.19, or at least 0.2 milligrams/kg/hour of an active ingredient such as ketamine. In some embodiments, a continuous infusion dose comprises an infusion rate range that is at least 0.0001, 0.001, 0.002, 0.003, 0.004, 0.005, 0.006, 0.007, 0.008, 0.009, 0.01, 0.015, 0.20, 0.025, 0.03, 0.035, 0.040, 0.045, 0.050, 0.055, 0.060, 0.065, 0.070, 0.075, 0.080, 0.085, 0.090, 0.095, 0.1, 0.11, 0.12, 0.13, 0.14, 0.15, 0.16, 0.17, 0.18, 0.19, or at least 0.2 milligrams/kg/hour and no more than 0.0001, 0.001, 0.002, 0.003, 0.004, 0.005, 0.006, 0.007, 0.008, 0.009, 0.01, 0.015, 0.20, 0.025, 0.03, 0.035, 0.040, 0.045, 0.050, 0.055, 0.060, 0.065, 0.070, 0.075, 0.080, 0.085, 0.090, 0.095, 0.1, 0.11, 0.12, 0.13, 0.14, 0.15, 0.16, 0.17, 0.18, 0.19, or at least 0.2 milligrams/kg/hour of an active ingredient such as ketamine.

In some embodiments, a programmed dosage regimen has an infusion duration (e.g., time to infuse a single dose). In some embodiments, a programmed dosage regimen has an infusion duration of at least 1, 2, 3, 4, 5, 6, 7, 8, 9, 10, 15, 20, 25, 30, 35, 40, 45, 50, 55, or 60 or more minutes, or at least 1, 1.5, 2.0, 2.5, 3.0, 3.5, 4.0, 4.5, 5.0, 6.0, 7.0, 8.0, 9.0, 11.0, 12.0, 13.0, 14.0, 15.0, 16.0, 17.0, 18.0, 19.0, 20.0, 21.0, 22.0, 23.0, or at least 24.0 hours or more, or at least 1, 2, 3, 4, 5, 6, 7, 8, 9, or 10 days or more. In some embodiments, a programmed dosage regimen has an infusion duration of no more than 1, 2, 3, 4, 5, 6, 7, 8, 9, 10, 15, 20, 25, 30, 35, 40, 45, 50, 55, or 60 minutes or more, or no more than 1, 1.5, 2.0, 2.5, 3.0, 3.5, 4.0, 4.5, 5.0, 6.0, 7.0, 8.0, 9.0, 11.0, 12.0, 13.0, 14.0, 15.0, 16.0, 17.0, 18.0, 19.0, 20.0, 21.0, 22.0, 23.0, or 24.0 hours or more, or no more than 1, 2, 3, 4, 5, 6, 7, 8, 9, or 10 days or more. In some embodiments, a programmed dosage regimen has an infusion duration that lasts until the drug formulation is depleted or almost depleted (e.g., over 80%, 85%, 90%, 95%, or 99% of the drug formulation in the drug reservoir or cartridge is depleted).

In some embodiments, a programmed dosage regimen comprises one or more doses. In some embodiments, a programmed dosage regimen comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 doses or more. In some embodiments, a programmed dosage regimen comprises no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 doses or more. In some embodiments, a programmed dosage regimen comprises an infusion rate range that is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 doses or more and no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 doses or more.

In some embodiments, a programmed dosage regimen comprises one or more doses per time period. In some embodiments, a programmed dosage regimen comprises at least 1, 2, 3, 4, 5, 6, 7, 8, 9, 10, 11, 12, 13, 14, 15, 16, 17, 18, 19, 20, 21, 22, 23, or 24 doses or more per 1, 2, 3, 4, 5, or 6 days, or per 1, 2, 3, 4, 5, 6, 7, or 8 weeks.

In some embodiments, a programmed dosage regimen has a treatment duration. For example, a treatment duration can be a month long treatment. In some embodiments, the treatment duration is indefinite (e.g., no set duration). In some embodiments, a programmed dosage regimen has a duration of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days or more. In some embodiments, a programmed dosage regimen has a duration of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks or more. In some embodiments, a programmed dosage regimen has a duration of no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days or more. In some embodiments, a programmed dosage regimen has a duration of no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks or more. In some embodiments, a programmed dosage regimen has a duration of between 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days and 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, a programmed dosage regimen has a duration of between 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks and 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks. In some embodiments, a continuous infusion dose comprises an infusion rate range that is at least 0.01, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or at least 200 mg/hour and no more than 0.01, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 125, 130, 140, 150, 160, 170, 180, 190, or no more than 200 mg/hour of an active ingredient such as ketamine. In some embodiments, a continuous infusion dose is delivered at more than one infusion rate during the duration of the dose. In some embodiments, a continuous infusion dose is delivered at a variable infusion rate. In some embodiments, a continuous infusion dose is delivered at an infusion rate that is optionally variable by the subject (e.g., subject can adjust the infusion rate while the dose is being administered). In some embodiments, a continuous infusion dose is interruptible by the subject such as pausing or turning off the dosage regimen and/or device. For example, in some embodiments, a continuous infusion dose comprises a duration when the infusion rate is 0.0 mg/hour.

In some embodiments, a programmed dosage regimen comprises one or more dosage limits. For example, a programmed dosage regimen may be locked to allow a user some flexibility to adjust a dosage or infusion rate within preset thresholds set by the authorized user or doctor/healthcare provider. Accordingly, a doctor may set a ketamine infusion threshold between 0.1 mg/kg and 1 mg/kg within which a user can adjust his infusion rate, but is unable to reconfigure the dosage regimen itself (e.g., adjust the thresholds). In some embodiments, a programmed dosage regimen comprises an upper limit setting a maximum quantity of a drug formulation to be delivered. In some embodiments, a programmed dosage regimen comprises a lower limit setting a minimum quantity of a drug formulation to be delivered. In some embodiments, a dosage limit is configured by a doctor or healthcare provider. In some embodiments, a dosage limit is configured by an authorized user or a user who provides authentication information for unlocking a drug delivery device. In some embodiments, a programmed dosage regimen comprises a single dose limit (e.g., limit amount of drug delivered in a single dose). In some embodiments, a single dose limit is about 0.01, 0.1, 0.2, 0.25, 0.3, 0.4, 0.5, 0.6, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 125, 150, 200, 250, 300, 350, 400, 450, or at least 500 mg per dose. In some embodiments, a single dose limit is at least 0.01, 0.1, 0.2, 0.25, 0.3, 0.4, 0.5, 0.6, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 125, 150, 200, 250, 300, 350, 400, 450, or at least 500 mg per dose and/or is no more than 0.01, 0.1, 0.2, 0.25, 0.3, 0.4, 0.5, 0.6, 0.7, 0.75, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, 10, 11, 12, 13, 14, 15, 16, 17, 18, 19, 20, 21, 22, 23, 24, 25, 30, 35, 40, 45, 50, 60, 70, 80, 90, 100, 125, 150, 200, 250, 300, 350, 400, 450, or at least 500 mg per dose.

In some embodiments, a single dose limit is about 0.001, 0.005, 0.006, 0.007, 0.008, 0.009, 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.60, 0.70, 0.80, 0.90, 1.0, 1.1, 1.2, 1.25, 1.3, 1.4, 1.50, 1.6, 1.7, 1.75, 1.8, 1.9, 2.00, 2.50, 3.00, 3.50, 4.00, 4.50, or at least 5.00 milligrams/kg/dose. In some embodiments, a single dose limit is about 1 milligrams/kg/dose. In some embodiments, a single dose limit is about 5 milligrams/kg/dose. In some embodiments, a single dose limit is at least 0, 0.005, 0.006, 0.007, 0.008, 0.009, 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.60, 0.70, 0.80, 0.90, 1.0, 1.1, 1.2, 1.25, 1.3, 1.4, 1.50, 1.6, 1.7, 1.75, 1.8, 1.9, 2.00, 2.50, 3.00, 3.50, 4.00, 4.50, or at least 5.00 milligrams/kg/dose and/or is no more than 0, 0.005, 0.006, 0.007, 0.008, 0.009, 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.60, 0.70, 0.80, 0.90, 1.0, 1.25, 1.50, 2.00, 2.50, 3.00, 3.50, 4.00, 4.50, or no more than 5.00 milligrams/kg/dose.

In some embodiments, a programmed dosage regimen comprises a daily dose limit (e.g., limit amount of drug delivered in a single day or 24 h). In some embodiments, a daily dose limit is about 0, 1, 2, 3, 4, 5, 10, 15, 20, 25, 30, 35, 40, 45, 50, 55, 60, 65, 70, 75, 80, 85, 90, 95, 100, 125, 150, 200, 250, 300, 350, 400, 450, or at least 500 mg per day. In some embodiments, a daily dose limit is at least 0, 1, 2, 3, 4, 5, 10, 15, 20, 25, 30, 35, 40, 45, 50, 55, 60, 65, 70, 75, 80, 85, 90, 95, 100, 125, 150, 200, 250, 300, 350, 400, 450, or at least 500 mg per day and/or is no more than 0, 1, 2, 3, 4, 5, 10, 15, 20, 25, 30, 35, 40, 45, 50, 55, 60, 65, 70, 75, 80, 85, 90, 95, 100, 125, 150, 200, 250, 300, 350, 400, 450, or no more than 500 mg per day. In some embodiments, a daily dose limit is about 125 mg per day. In some embodiments, a daily dose limit is about 200 mg per day.

In some embodiments, a daily dose limit is about 0, 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.10, 1.20, 1.25, 1.30, 1.40, 1.50, 1.60, 1.70, 1.80, 1.90, 2.00, 2.50, 3.00, 3.50, 4.00, 4.50, or at least 5.00 milligrams/kg/day. In some embodiments, a daily dose limit is about 1 mg/kg/day. In some embodiments, a daily dose limit is about 5 mg/kg/day. In some embodiments, a daily dose limit is at least 0, 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.10, 1.20, 1.25, 1.30, 1.40, 1.50, 1.60, 1.70, 1.80, 1.90, 2.00, 2.50, 3.00, 3.50, 4.00, 4.50, or at least 5.00 milligrams/kg/day and/or is no more than 0, 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.10, 1.20, 1.25, 1.30, 1.40, 1.50, 1.60, 1.70, 1.80, 1.90, 2.00, 2.50, 3.00, 3.50, 4.00, 4.50, or at least 5.00 milligrams/kg/day.

In some embodiments, a programmed dosage regimen comprises a weekly dose limit (e.g., limit amount of drug delivered in a single week or 7 days). In some embodiments, a weekly dose limit is about 1, 5, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, or at least 1000 mg per week. In some embodiments, a weekly dose limit is at least 1, 5, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, or at least 1000 mg per week and/or is no more than 1, 5, 10, 15, 20, 25, 30, 35, 40, 45, 50, 60, 70, 80, 90, 100, 125, 150, 200, 250, 300, 350, 400, 450, 500, 550, 600, 650, 700, 750, 800, 850, 900, 950, or no more than 1000 per week.

In some embodiments, a weekly dose limit is about 0.25, 0.5, 0.75, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 125, 150, 200, 250, 300, 350, 400, 450, or at least 500 milligrams/kg/week. In some embodiments, a weekly dose limit is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, or at least 500 mg/kg/week and/or is no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 125, 150, 200, 250, 300, 350, 400, 450, or no more than 500 mg/kg/week.

In some embodiments, a programmed dosage regimen provides a clinically effective steady state plasma concentration of the active ingredient such as ketamine within 1, 2, 3, 4, 5, 6, 7, 8, 9, 10, 11, 12, 13, 14, 15, 16, 17, 18, 19, 20, 21, 22, 23, 24 hours or more of treatment. In some embodiments, a programmed dosage regimen provides a clinically effective steady state plasma concentration of an active ingredient such as ketamine within 1, 2, 3, 4, 5, 6, 7, 8, 9, 10, 11, 12, 13, 14, 15, 16, 17, 18, 19, 20, 21, 22, 23, 24 hours or more of treatment outside of a hospital or clinic environment. In some embodiments, a programmed dosage regimen provides a steady state drug plasma concentration (e.g., ketamine) of at least 1, 2, 3, 4, 5, 6, 7, 8, 9, 10, 15, 20, 25, 30, 35, 40, 45, 50, 60, 70, 80, 90, 100, 125, 150, 175, 200, 250, 300, 350, 400, 450, 500, 550, 600, 650, 700, 750, 800, 850, 900, 950, 1000, 1500, 2000, 2500, 3000, 3500, 4000, 4500, 5000, 6000, 7000, 8000, 9000, or 10000 or more ng/mL and/or no more than 1, 2, 3, 4, 5, 6, 7, 8, 9, 10, 15, 20, 25, 30, 35, 40, 45, 50, 60, 70, 80, 90, 100, 125, 150, 175, 200, 250, 300, 350, 400, 450, 500, 550, 600, 650, 700, 750, 800, 850, 900, 950, 1000, 1500, 2000, 2500, 3000, 3500, 4000, 4500, 5000, 6000, 7000, 8000, 9000, or 10000 or more ng/mL.

In some embodiments, a programmed dosage regimen provides a clinically effective steady state plasma concentration of an active ingredient such as ketamine with a peak trough fluctuation that is lower than a comparable fluctuation from intravenous or intramuscular administration in a hospital or clinic setting. In some embodiments, a programmed dosage regimen provides a continuous infusion or a series of doses that reduce the fluctuation between the peak and trough plasma concentrations of the active ingredient. In some embodiments, a programmed dosage regimen provides a clinically effective steady state plasma concentration of an active ingredient such as ketamine with a peak trough fluctuation of no more than 5%, 10%, 20%, 30%, 40%, 50%, 60% 70%, 80%, 90%, 100%, 150%, 200%, 250%, 300%, 350%, 400%, 450%, 500%, 600%, 700%, 800%, 900%, or 1000% or more of the average steady state concentration during treatment. In some embodiments, a programmed dosage regimen provides a clinically effective steady state plasma concentration of an active ingredient such as ketamine with a peak to trough ratio of no more than 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, or 10.0 or more.

In some embodiments, a programmed dosage regimen provides an effective steady state drug plasma concentration (e.g., ketamine) while providing relatively low peak trough fluctuation. In some embodiments, a programmed dosage regimen provides a steady state drug plasma concentration of at least 1, 2, 3, 4, 5, 6, 7, 8, 9, 10, 15, 20, 25, 30, 35, 40, 45, 50, 60, 70, 80, 90, 100, 125, 150, 175, 200, 250, 300, 350, 400, 450, 500, 550, 600, 650, 700, 750, 800, 850, 900, 950, 1000, 1500, 2000, 2500, 3000, 3500, 4000, 4500, 5000, 6000, 7000, 8000, 9000, or 10000 or more ng/mL and/or a peak to trough ratio of no more than 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, or 10.0 or more.

In some embodiments, a programmed dosage regimen provides a steady state drug plasma concentration (e.g., ketamine) having a Cₘₐₓ of no more than 1, 2, 3, 4, 5, 6, 7, 8, 9, 10, 15, 20, 25, 30, 35, 40, 45, 50, 60, 70, 80, 90, 100, 125, 150, 175, 200, 250, 300, 350, 400, 450, 500, 550, 600, 650, 700, 750, 800, 850, 900, 950, 1000, 1500, 2000, 2500, 3000, 3500, 4000, 4500, 5000, 6000, 7000, 8000, 9000, or 10000 or more ng/mL, and/or a Cₘᵢₙ of at least 1, 2, 3, 4, 5, 6, 7, 8, 9, 10, 15, 20, 25, 30, 35, 40, 45, 50, 60, 70, 80, 90, 100, 125, 150, 175, 200, 250, 300, 350, 400, 450, 500, 550, 600, 650, 700, 750, 800, 850, 900, 950, 1000, 1500, 2000, 2500, 3000, 3500, 4000, 4500, 5000, 6000, 7000, 8000, 9000, or 10000 or more ng/mL.

In some embodiments, a programmed dosage regimen provides an effective steady state drug plasma concentration (e.g., ketamine) having a Cₘₐₓ to Cₘᵢₙ ratio of no more than 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, 10.0, 11, 12, 13, 14, 15, 16, 17, 18, 19, 20, 25, 30, 35, 40, 45, or 50 or more.

### Pharmaceutical Compositions

The terms “pharmaceutical composition” and “drug formulation,” as used herein, are synonymous.

In an aspect, provided herein is a pharmaceutical composition, comprising:
(i) an NMDA receptor antagonist or modulator, or a hydrate, solvate, or pharmaceutically acceptable salt thereof; and
(ii) at least one pharmaceutically acceptable excipient, wherein the pharmaceutical composition is in a form for dosing or administration by intravenous (I. V.), intramuscular, subcutaneous, or intradermal injection.

In some embodiments, the drug formulation or pharmaceutical composition administered according to the systems, devices, kits, formulations, and methods disclosed herein is a liquid formulation such as an aqueous solution. In some embodiments, the formulation or pharmaceutical composition is configured to be administered by intramuscular injection. In some embodiments, the formulation or pharmaceutical composition is configured to be administered by subcutaneous injection. In some embodiments, the formulation or pharmaceutical composition is configured to be administered by intravenous injection. In some embodiments, the formulation or pharmaceutical composition is administered continuously as an infusion. In some embodiments, the formulation or pharmaceutical composition is administered by injection as a bolus. In some embodiments, the formulation or pharmaceutical composition is administered by injection as a bolus over a period of time such as about 10 minutes.

In some embodiments, the formulation is configured to be administered through a pump device, as described herein.

In certain embodiments of the pharmaceutical compositions described herein, the at least one pharmaceutically acceptable excipient is (i) a surface-active agent, (ii) a non-ionic surfactant, (iii) a phospholipid solubilization agent, (iv) a cyclodextrin excipient, (v) an emulsion stabilizer, (vi) a preservative, (vii) an antimicrobial agent, or (viii) a topical analgesic. In some embodiments, the topical analgesic is lidocaine.

In certain embodiments of the pharmaceutical compositions described herein, the dosage form is an I.V. dosage form.

In certain embodiments of the pharmaceutical compositions described herein, the NMDA receptor antagonist or modulator is an arylcyclohexylamine or arylcyclohexylamine derivative.

In certain embodiments of the pharmaceutical compositions described herein, the NMDA receptor antagonist or modulator also acts as a dopamine reuptake inhibitor, μ-opioid receptor agonist, σ receptor agonist, nACh receptor antagonist, D2 receptor agonistic, or any combination thereof.

In certain embodiments of the pharmaceutical compositions described herein, the NMDA receptor antagonist or modulator is ketamine, phencyclidine (PCP), 3-MeO-Phencylidine, 4-MeO-Phencyclidine, eticyclidine (PCE), 3-MeO-PCE, methoxetamine (MXE), tiletamine, or tenocyclidine (TCP), or a hydrate, solvate, or pharmaceutically acceptable salt thereof.

In certain embodiments, the pharmaceutical composition comprises from about 10 mg/mL to about 300 mg/mL of the NMDA receptor antagonist, or a hydrate, solvate, or pharmaceutically acceptable salt thereof.

In certain embodiments, the pharmaceutical composition comprises from about 10 mg/mL to about 50 mg/mL of the NMDA receptor antagonist or modulator, or a hydrate, solvate, or pharmaceutically acceptable salt thereof.

In certain embodiments, the pharmaceutical composition comprises about 10 mg/mL, about 15 mg/mL, about 20 mg/mL, about 25 mg/mL, about 30 mg/mL, about 35 mg/mL, about 40 mg/mL, about 45 mg/mL, or about 50 mg/mL of ketamine, or a hydrate, solvate, or pharmaceutically acceptable salt thereof.

In certain embodiments, the pharmaceutical composition comprises up to about 300 mg/mL of ketamine, or a hydrate, solvate, or pharmaceutically acceptable salt thereof.

In certain embodiments, the pharmaceutical composition comprises a pH of about 3.5 to 7.5.

In certain embodiments, the pharmaceutical composition comprises a pH of about 5.5 to 7.0.

In certain embodiments, the pharmaceutical composition comprises a pH of about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, or about 7.0.

In certain embodiments, the dosage form or pharmaceutical composition comprises a co-solvent.

In certain embodiments of the pharmaceutical compositions described herein, the co-solvent comprises PEG200, PEG300, PEG400, PEG600, propylene glycol, ethanol, polysorbate 20, polysorbate 80, cremephor, glycerin, benzyl alcohol, dimethylacetamide (DMA), N-methyl-2-pyrrolidone (NMP), tert-butanol, or combinations thereof.

In certain embodiments, the dosage form or pharmaceutical composition comprises a surface-active agent.

In certain embodiments of the pharmaceutical compositions described herein, the surface-active agent comprises polyoxyethylene sorbitan monooleate (Tween 80), sorbitan monooleate, polyoxyethylene sorbitan monolaurate (Tween 20), lechitin, polyoxyethylene-polyoxypropylene copolymers (Pluronics1), or combinations thereof.

In certain embodiments, the dosage form or pharmaceutical composition comprises a non-ionic surfactant.

In certain embodiments of the pharmaceutical compositions described herein, the non-ionic surfactant comprises Cremophor RH40, Cremophor RH60, d-alpha-topopherol polyethylene glycol 1000 succinate, polysorbate 20, polysorbate 80, Solutol HS 15, sorbitan monooleate, poloxamer 407, Labrafil M-1944CS, Labrafil M-2125CS, Labrasol, Gellucire 44/14, Softigen 767, or combinations thereof.

In certain embodiments of the pharmaceutical compositions described herein, the NMDA receptor antagonist or modulator is racemic ketamine, (R)-ketamine, or (S)-ketamine.

In some embodiments, the pharmaceutical composition comprises one or more co-solvents, solubilization/solubilizing agents, stabilization agents, antioxidants, preservatives, cryoprotectants, lyoprotectants, bulking agents, tonicity-adjusting agents, or antimicrobial agents. In some embodiments, the pharmaceutical composition comprises at least one co-solvent. In some embodiments, the pharmaceutical composition comprises at least one solubilizing agent. In some embodiments, the pharmaceutical composition comprises at least one stabilization agent. In some embodiments, the pharmaceutical composition comprises at least one antioxidant. In some embodiments, the pharmaceutical composition comprises at least one preservative. In some embodiments, the pharmaceutical composition comprises at least one cryoprotectant. In some embodiments, the pharmaceutical composition comprises at least one lyoprotectant. In some embodiments, the pharmaceutical composition comprises at least one bulking agent. In some embodiments, the pharmaceutical composition comprises at least one tonicity-adjusting agent. In some embodiments, the pharmaceutical composition comprises at least one antimicrobial agent.

In some embodiments, the formulation or pharmaceutical composition has a pH of about 1 to 2, about 2 to 3, about 3 to 4, about 4 to 5, about 5 to 6, about 6 to 7, about 7 to 8, about 8 to 9, about 9 to 10, about 10 to 11, about 11 to 12, about 12 to 13, or about 13 to 14. In some embodiments, the formulation comprises ketamine and has a slightly acidic pH. In some embodiments, a formulation comprising ketamine has a pH of about 3.5 to about 5.5. In some embodiments, a formulation comprising ketamine has a pH of about 1 to about 3, about 2 to about 4, about 3 to about 5, about 4 to about 6, or about 5 to about 7. In some embodiments, a formulation comprising ketamine has a pH of about 3.0, about 3.5, about 4.0, about 4.5, about 5.0, about 5.5, about 6.0, about 6.5, or about 7. In some embodiments, a formulation comprising ketamine has a pH of about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, or about 7.0.

In some embodiments, the formulation or pharmaceutical composition comprises an active ingredient such as ketamine at a concentration of at least about 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 350, 400, 450, or 500 mg/mL or more and/or no more than about 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 350, 400, 450, or 500 mg/mL or more. In some embodiments, the formulation comprises an active ingredient such as ketamine at a concentration of about 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 350, 400, 450, or 500 mg/mL or more. In some embodiments, the formulation comprises an active ingredient such as ketamine at a concentration of about 10 mg/mL to about 300 mg/mL.

In some embodiments, the formulation or pharmaceutical composition is a pharmaceutical composition. In some embodiments, the formulation is in the form of a sterile injectable aqueous or oleaginous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor® EL (BASF, Parsippany, N.J.) or phosphate buffered saline (PBS), ethanol, polyol (e.g., glycerol, propylene glycol, and liquid polyethylene glycol), and suitable mixtures thereof. In addition, sterile fixed oils are conventionally employed as a solvent or suspending medium; for this purpose, any bland fixed oil may be employed, including synthetic mono- or diglycerides. Moreover, fatty acids, such as oleic acid, find use in the preparation of injectables. Prolonged absorption of particular injectable formulations can be achieved by including an agent that delays absorption (e.g., aluminum monostearate or gelatin). In some embodiments, the formulation comprises a co-solvent. In some embodiments, a suitable co-solvent is propylene glycol, glycerin, ethanol, polyethylene glycol (300 and 400), Sorbitol, dimethylacetamide, Cremophor EL, or N-methyl-2-pyrrolidone, or dimethylsulfoxide.

In some embodiments, the formulation or pharmaceutical composition is an aqueous suspension. Aqueous suspensions contain active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives (e.g. benzethonium chloride).

In some embodiments, the formulation or pharmaceutical composition comprises a stabilization agent. In some embodiments, the formulation comprises a surface-active solubilization agent. Surface-active solubilization agents include, but are not limited to: polyoxyethylene sorbitan monooleate (Tween 80), sorbitan monooleate, polyoxyethylene sorbitan monolaurate (Tween 20), lecithin, and Polyoxyethylene-polyoxypropylene copolymers (Pluronics1). In some embodiments, the formulation comprises a non-ionic surfactant solubilization agent. Non-ionic surfactants include, but are not limited: Cremophor RH 40, Cremophor RH 60, d-alpha-tocopherol polyethylene glycol 1000 succinate, polysorbate 20, polysorbate 80, Solutol HS 1, sorbitan monooleate, poloxamer 407, Labrafil M-1944CS, Labrafil M-2125CS, Labrasol, Gellucire 44/14, Softigen 767, and mono-fatty esters and di-fatty acid esters of PEG 300, 400, and 1750. In some embodiments, the formulation comprises a phospholipid solubilizing agent such as, hydrogenated soy phosphatidylcholine, phosphatidylcholine, distearoylphosphatidylglycerol, L-alpha-dimyristoylphosphatidylcholine, or L-alpha-dimyristoylphosphatidylglycerol.

In some embodiments, the formulation or pharmaceutical composition comprises a complexation agent. In some embodiments, the complexation agent is hydroxypropyl-b-cyclodextrin, bulfobutylether-b-cyclodextrin (Captisol1), or polyvinylpyrrolidone. In some embodiments, the complexation agent is an amino acid such as, arginine, lysine, or histidine.

In some embodiments, the formulation or pharmaceutical composition comprises a cyclodextrin excipient. Cyclodextrin excipients are used to enhance the stability, tolerability and absorption of compounds in parenteral aqueous solutions. Common cyclodextrin excipients include but are not limited to: alpha-Cyclodextrin (alpha-CD), beta-Cyclodextrin (beta-CD), gamma-Cyclodextrin (gamma-CD), Diethyl-ethyl-beta-cyclodextrin (DE-beta-CD), Dimethyl-ethyl-beta-cyclodextrin (DM-beta-CD), Hydroxypropyl-beta-cyclodextrin (HP-beta-CD), Hydroxypropyl-gamma-cyclodextrin (HP-gamma-CD), Methyl-b-cyclodextrin (M-beta-CD), Sulfobutylether-beta-cyclodextrin (SBE-beta-CD), Randomly methylated-beta-CD (RM-beta-CD), Maltosyl-beta-CD (mal-beta-CD), Hydroxypropyl-alpha-CD.

The formulations or pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The formulation or pharmaceutical composition typically comprises a therapeutically effective amount of an active compound, such as ketamine, or a hydrate, solvate, tautomer, or pharmaceutically acceptable salt thereof, and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate-buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a triethanolamine (Tris) buffer, histidine, bicarbonate; N-(2-Hydroxyethyl)piperazine-N′-(2-ethanesulfonic acid) (HEPES); 2-(N-Morpholino)ethanesulfonic acid (MES); 2-(N-Morpholino)ethanesulfonic acid sodium salt (YMS); 3-(N-Morpholino)propanesulfonic acid (MOPS); and N-tris[Hydroxymethyl]methyl-3-aminopropanesulfonic acid (TAPS).

Many active pharmaceutical ingredients (APIs) are weak acids or weak bases. Weak acids or weak bases can exist in an un-ionized form or as an ionized complex prepared by the addition of an base or acid respectively. The resultant complex is stabilized by ionic interactions and is known as a salt. This complex exists via an ionic bond between an ionized API and an oppositely charged counterion. Salts offer a number of advantages over their un-ionized counterparts. The choice of counterion can have a large influence on the salts properties and the use of a given salt form of a given API in a pharmaceutical product is influenced and guided by a number of factors for example stability (photo, hydrolytic and thermal), solubility, physicochemical properties, solid state properties (crystallinity, polymorphism, particle size, crystal morphology, melting point, compactability), production considerations (e.g., ease of handling and processing), dissolution rate, modulation of drug release, compatibility with excipients and containers, ease and consistency of production, desired route of administration, and organoleptic factors (e.g., taste). Furthermore with respect to injection, salt can influence pain and irritation at the injection site (Brazeau et al. 1998).

With regards to cyclodextrin solubilization, specific salts of various APIs have been found to form multicomponent complexes/systems or ternary systems which can have distinct desirable properties as compared to their standard binary complexes/systems counterparts prepared between the cyclodextrin and the un-ionized API, as well as compared to other multicomponent ternary complexes/systems involving different salt forms of that API. (Kim et al. 1998, Mura et al. 1999, Mura et al. 1999, Redenti et al. 2000, Ribeiro et al. 2005). These multicomponent complexes/systems can thus dramatically influence solubility of the API in aqueous solutions, dissolution rates, can influence product stability, and pharmacokinetic properties of the pharmaceutical preparation.

In some embodiments, disclosed herein are formulations that have an acid added to the API. In some embodiments, a non-stoichiometric amount of acid is added to a solution of ketamine and excipients (such as cyclodextrins or emulsifiers, etc.) to obtain a clear and soluble solution in the desired pH range. In some embodiments, this would preferably include a molar equivalent of 0.5-1.0 of acid relative to ketamine. In other embodiments, this would preferably include a molar equivalent of 0.1-0.4 molar equivalents of acid relative to ketamine. In some embodiments, a molar equivalent of at least about 0.1, 0.2, 0.3, 0.4, 0.5, 0.6, 0.7, 0.8, 0.9, or 1.0 of acid relative to the API (e.g., ketamine) and/or no more than about 0.1, 0.2, 0.3, 0.4, 0.5, 0.6, 0.7, 0.8, 0.9, or 1.0 is added to a solution of the API and excipients.

Examples of the acid include hydrochloric acid, hydrobromic acid, hydroiodic acid, nitric acid, aspartic acid, carbonic acid, sulfuric acid, phosphoric acid, acetic acid, malic acid, maleic acid, lactic acid, tartaric acid, citric acid, succinic acid, decanoic acid, propanoic acid, fumaric acid, gluconic acid, glucuronic acid, trifluoroacetic acid, glutamic acid, mucic acid, formic acid, mandelic acid, hippuric acid, pamoic acid, oleic acid, methansulfonic acid, ethanesulfonic acid, p-toluenesulfonic acid, benzensulfonic acid, naphthalenesulfonic acid, isethionic acid, camphorsulfonic acid, methylsulfuric acid, benzoic acid, saccharic acid, naphthoic acid, salicylic acid, nicotinic acid, laurylsulfuric acid, stearic acid, pyroglutamic acid, and sulfosalicylic acid.

In some formulations, a pharmaceutically acceptable salt of the API (e.g., ketamine) is used to prepare the solution or formulation.

Examples of salts of the API include hydrochloride, hydrobromide, hydroiodide, acetate, aspartate, benzoate, besylate, camphorsulfonate, citrate, carbonate, decanoate, ethandisulfonate, fumarate, formate, gluconate, glucoronate, glutamate, hippurate, isethionate, lactate, laurylsulfate, malate, maleate, mandelate, mesylate, methylsulfate, mucate, napthoate, napsylate, nitrate, oleate, pamoate, phosphate, propionate, saccharate, succinate, sulfate, sulfosalicylate, tartrate, tosylate, trifluoroactate nicotinate, salicylate, stearate, and pyroglutamate.

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampule, syringe, or autoinjector (similar to, e.g., an EpiPen®)), whereas a multi-use container (e.g., a multi-use vial) is provided in other embodiments.

Formulations or pharmaceutical compositions can also include carriers to protect the composition against rapid degradation or elimination from the body, such as a controlled release formulation, including liposomes, hydrogels, prodrugs and microencapsulated delivery systems. For example, a time-delay material such as glyceryl monostearate or glyceryl stearate alone, or in combination with a wax, may be employed. The drug delivery devices described herein may be used to deliver the formulations.

In some embodiments, the formulation or pharmaceutical composition is stored in a reservoir of the drug delivery device. In some embodiments, the formulation is stored in a cartridge that is insertable and/or attachable to the drug delivery device. In some embodiments, the cartridge and/or drug delivery device comprises a product label for intramuscular injection. In some embodiments, the cartridge and/or drug delivery device comprises a product label for subcutaneous injection. In some embodiments, the cartridge and/or drug delivery device comprises a product label for intravenous injection. In some embodiments, disclosed herein is a kit comprising a product label for intramuscular injection. In some embodiments, disclosed herein is a kit comprising a product label for subcutaneous injection. In some embodiments, disclosed herein is a kit comprising a product label for intravenous injection.

In some embodiments, the formulation or pharmaceutical composition is a liquid formulation comprising ketamine hydrochloride (HCl). In some embodiments, the formulation comprises a racemic ketamine composition. Alternatively, in some embodiments, the formulation comprises a substantially pure stereoisomer of ketamine (e.g., over 90%, 95%, 96%, 97%, 98%, or 99% of the ketamine is one stereoisomer). In some embodiments, the formulation comprises substantially pure S-ketamine. In some embodiments, the formulation comprises substantially pure R-ketamine. In some embodiments, the NMDA receptor antagonist is at least about 90%, about 95%, about 96%, about 97%, about 98%, or about 99% pure. In some embodiments, the NMDA receptor antagonist is at least about 99.1%, about 99.2%, about 99.3%, about 99.4%, about 99.5%, about 99.6%, about 99.8%, or about 99.9% pure. In some embodiments, the NMRA receptor antagonist comprises less than about 5%, about 4%, about 3%, about 2%, or about 1% impurities.

It is frequently beneficial to improve one of more physical properties of the treatment modalities disclosed herein and/or the manner in which they are administered. Improvements of physical properties include, for example, methods of increasing water solubility, bioavailability, serum half-life, and/or therapeutic half-life; and/or modulating biological activity. Modifications known in the art include pegylation, Fc-fusion and albumin fusion. Although generally associated with large molecule agents (e.g., polypeptides), such modifications have recently been evaluated with particular small molecules. By way of example, Chiang, M. et al. (J. Am. Chem. Soc., 2014, 136(9):3370-73) describe a small molecule agonist of the adenosine 2a receptor conjugated to the immunoglobulin Fc domain. The small molecule-Fc conjugate retained potent Fc receptor and adenosine 2a receptor interactions and showed superior properties compared to the unconjugated small molecule. Covalent attachment of PEG molecules to small molecule therapeutics has also been described (Li, W. et al., Progress in Polymer Science, 2013 38:421-44).

The NMDA receptor antagonist of the present disclosure may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED₅₀ of an agent is the dose or amount of an agent that produces a therapeutic response or desired effect in 50% of the population to which it is administered. Although the ED₅₀ is commonly used as a measure of reasonable expectance of an agent's effect, it is not necessarily the dose that a clinician might deem appropriate taking into consideration all relevant factors. Thus, in some situations the effective amount is more than the calculated ED₅₀, in other situations the effective amount is less than the calculated ED₅₀, and in still other situations the effective amount is the same as the calculated ED₅₀.

In addition, an effective dose of the NMDA receptor antagonist of the present disclosure may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In embodiments, the dosage of the NMDA receptor antagonist is contained in a “unit dosage form.” The phrase “unit dosage form” refers to physically discrete units, each unit including a predetermined amount of the compound (e.g., ketamine, or a hydrate, solvate, or pharmaceutically acceptable salt thereof), sufficient to produce the desired effect. It will be appreciated that the parameters of a unit dosage form will depend on the particular agent and the effect to be achieved.

### Combination Therapy

In certain instances, the NMDA receptor antagonist, or a hydrate, solvate, or pharmaceutically acceptable salt thereof, or a pharmaceutically acceptable salt thereof is administered in combination with a second therapeutic agent.

In some embodiments, the benefit experienced by a subject is increased by administering one of the compounds described herein with a second therapeutic agent (which also includes a therapeutic regimen) that also has therapeutic benefit. In some embodiments, the NMDA receptor antagonist composition, is co-administered with an additional therapeutic that mitigates and/or alleviates the side-effects of the NMDA receptor antagonist.

In any case, regardless of the disease, disorder or condition being treated, the overall benefit experienced by the subject is simply additive of the two therapeutic agents or the subject experiences a synergistic benefit.

In certain embodiments, different therapeutically-effective dosages of the compounds disclosed herein will be utilized in formulating a pharmaceutical composition and/or in treatment regimens when the compounds disclosed herein are administered in combination with a second therapeutic agent. Therapeutically-effective dosages of drugs and other agents for use in combination treatment regimens are optionally determined by means similar to those set forth hereinabove for the actives themselves. Furthermore, the methods of prevention/treatment described herein encompasses the use of metronomic dosing, i.e., providing more frequent, lower doses in order to minimize toxic side effects.

It is understood that the dosage regimen to treat, prevent, or ameliorate the condition(s) for which relief is sought, is modified in accordance with a variety of factors (e.g., the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated, and so forth. In additional embodiments, when co-administered with a second therapeutic agent, the compound provided herein is administered either simultaneously with the second therapeutic agent, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

In certain embodiments, the additional therapeutic is a second active agent. In some embodiments, the additional therapeutic is a benzodiazepine, a selective serotonin 5-HT3 receptor antagonist, a beta blocker, an inhibitor of CYP3D6 and/or CYP3A and/or CYP2C9, or a combination thereof. In some embodiments, the second therapeutic is a benzodiazepine. In some embodiments, the benzodiazepine is lorazepam or midazolam. In some embodiments, the second therapeutic is a beta blocker. In some embodiments, the beta blocker is propranolol or atenolol. In some embodiments, the second therapeutic is a selective 5-HT3 receptor antagonist. In some embodiments, the selective 5-HT3 receptor antagonist is ondansetron. In some embodiments, the second therapeutic is an inhibitor of CYP2B6 and/or CYP3A and/or CYP2C9. In some embodiments, the inhibitor of CYP2B6 is clopidogrel, ticlopidine, orphenadrine, candesartan, amlodipine, felodipine, memantine, clotrimazole, voriconazole, azelastine, clopidogrel, clofibrate, fenofibrate, 2-phenyl-2-(1-piperidinyl)propane, resveratrol, alpha-viniferin, epsilon-viniferin or pregabalin. In some embodiments, the inhibitor of CYP3A is nefazodone, aprepitant, fluvoxamine, itraconazole, verapamil, orphenadrine, bergamottin, mibefradil, ketoconazole, itraconazole, resveratrol, alpha-viniferin, epsilon-viniferin or diltiazem. In some embodiments, co-administration includes administering one active agent within 0.5, 1, 2, 4, 6, 8, 10, 12, 16, 20, 24 hours, 2 days, 4 days, 1 week or 1 month of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In certain embodiments of the pharmaceutical compositions described herein, the pharmaceutical composition further comprises at least one additional active agent that mitigates the side effects of the NMDA receptor antagonist. In certain embodiments of the pharmaceutical compositions described herein, the at least one additional active agent is a benzodiazepine, a selective serotonin 5-HT3 receptor antagonist, a beta blocker, an inhibitor of CYP2B6 and/or CYP3A and/or CYP2C9, or combinations thereof.

In certain embodiments of the pharmaceutical compositions described herein, the at least one additional active agent is a benzodiazepine, a selective serotonin 5-HT3 receptor antagonist, or a beta blocker. In certain embodiments of the pharmaceutical compositions described herein, the benzodiazepine is lorazepam or midazolam. In certain embodiments of the pharmaceutical compositions described herein, the beta blocker is propranolol or atenolol. In certain embodiments of the pharmaceutical compositions described herein, the selective 5-HT3 receptor antagonist is ondansetron. In certain embodiments of the pharmaceutical compositions described herein, the at least one additional active agent is an inhibitor of CYP2B6 and/or CYP3A and/or CYP2C9.

In certain embodiments of the pharmaceutical compositions described herein, the inhibitor of CYP2B6 is clopidogrel, ticlopidine, orphenadrine, candesartan, amlodipine, felodipine, memantine, clotrimazole, voriconazole, azelastine, clopidogrel, clofibrate, fenofibrate, 2-phenyl-2-(1-piperidinyl)propane, resveratrol, alpha-viniferin, epsilon-viniferin or pregabalin.

In certain embodiments of the pharmaceutical compositions described herein, the inhibitor of CYP3A is nefazodone, aprepitant, fluvoxamine, itraconazole, verapamil, orphenadrine, bergamottin, mibefradil, ketoconazole, itraconazole, resveratrol, alpha-viniferin, epsilon-viniferin or diltiazem.

### Tamper Resistant Devices and Cartridges

Disclosed herein are systems, devices, and methods that provide tamper resistant features to prevent or reduce the risk of unauthorized use or abuse. In some embodiments, the tamper resistant features comprise safety features to prevent injury or harm. In some embodiments, tamper resistant features include physical or mechanical elements or properties designed to resist tampering such as attempts to penetrate the drug delivery device, drug reservoir, or drug cartridge (e.g., reinforced walls or surface).

In some embodiments, a tamper resistant feature comprises a sliding lock-off window that permanently secures the filling port on an internally integrated reservoir from any further access after it is filled by a pharmacist, or doctor, or a certified service, or a manufacturer.

In some embodiments, a tamper resistant feature a sliding lock-off window that secures the filling port on an internally integrated reservoir after it is filled by a pharmacist, or doctor, or a certified service, or a manufacturer in a fashion that is reversible with a physical key, or an electronic key, password or other biometric identification system.

In some embodiments, a tamper resistant feature comprises an internal or external locking system that secures a disposable drug reservoir from any further access after it is inserted by a pharmacist, or doctor, or a certified service or a manufacturer.

In some embodiments, a tamper resistant feature comprises an internal or external locking system that secures a disposable drug reservoir after it is inserted by a pharmacist, or doctor, or a certified service or a manufacturer in a fashion that is reversible with a physical key, or an electronic key, password or other biometric identification system programmed into the device.

In some embodiments, a tamper resistant feature comprises a self-contained motion detection system (e.g. accelerometer) or GPS related motion detection system. In some embodiments, the motion detection system is configured to monitor one or more biometric parameters such as movement, velocity and/or acceleration during certain treatment modes (e.g., bolus dosing) in order to detect non-sanctioned behavior (e.g., driving, walking, running). In some embodiments, detection of non-sanctioned behavior signals a potential need for modification of treatment parameters either automatically (e.g., as per firmware programming) or as per the discretion and/or direction of a remote treating physician or other certified person. In some embodiments, the modification comprises shutting down the device, locking off any further use without oversight, notifying the treating physician of potential non-sanctioned use, changing the delivery parameters remotely, or any combination thereof. In some embodiments, the systems, devices, and methods disclosed herein are configured to modify the treatment parameters upon detection of non-sanctioned behavior. In some embodiments, the modification occurs after a threshold number of incidents of non-sanctioned behavior have been detected. In some embodiments, the modification occurs after at least 1, 2, 3, 4, 5, 6, 7, 8, 9, or 10 or more incidents of non-sanctioned behavior have been detected.

In some embodiments, tamper resistant features provide inactivation and/or neutralization of the active ingredient of the drug formulation stored in the device and/or cartridge upon detection of a breach or tampering attempt. In some embodiments, a breach is detected based on a pressure change. In some embodiments, a breach triggers the release of one or more components configured to prevent unauthorized use of the liquid drug formulation. In some embodiments, a breach triggers the release of activated charcoal into the liquid drug formulation to absorb the active ingredient. In some embodiments, a breach triggers the release of a biocompatible gel forming polymer to convert the liquid drug formulation into a gel or solid (e.g., so as to reduce or prevent injection of the drug formulation). In some embodiments, a gel forming polymer is gellan gum, alginic acid, xyloglucan, pectin, chitosan, poly(DL-lactic acid), poly(DL-lactide-co-glycolide), or poly-caprolactone. In some embodiments, the drug delivery device and/or drug cartridge comprises a filter disposed between the liquid drug formulation and the injection site to prevent injection of one or more components solids or particles into the subject. For example, accidental damage to the drug delivery device or cartridge may cause activated charcoal to be released into the liquid drug formulation, but the presence of the filter prevents any of the charcoal from being injected into the patient.

In some embodiments, the drug delivery device and/or drug cartridge comprises a filter for filtering the liquid drug formulation. In some embodiments, the filter is a 0.1 micron filter. In some embodiments, the filter comprises a cellulose nitrate, cellulose acetate, nylon, polyether-sulfone, regenerate cellulose, or PTFE membrane. In some embodiments, the filter has a pore size of at least 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or at least 5.0 microns or more and/or a pore size of no more than 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or at least 5.0 microns or more. In some embodiments, the filter has a pore size of about 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or at least 5.0 microns or more. In some embodiments, the filter is a 0.8 micron filter. In some embodiments, the filter is a 0.45 micron filter. In some embodiments, the filter is a 0.2 micron filter. In some embodiments, the filter is a 0.22 micron filter.

In some embodiments, tamper resistant features include software restrictions on access to the dosage regimen or dosing parameters. For example, in some embodiments, a software restriction is a password authentication requirement for a user to configure or modify a dosage regimen or an individual dose. In some embodiments, a software restriction is a biometric authentication step required for a user to configure or modify a dosage regimen or an individual dose (e.g., via a fingerprint scanner on the drug delivery device). In some embodiments, a drug delivery device comprises at least one processor and instructions executable by the at least one processor to create an application comprising a software module carrying out an authentication step. In some embodiments, a drug delivery device comprises an authentication module for authenticating a user or authorized user. In some embodiments, an authentication module provides at least two levels of access. In some embodiments, an authentication module grants access for a user or subject to administer a dose according to a dosage regimen, but restricts or limits the ability to configure or modify the dosage regimen. In some embodiments, an authentication module grants access to an authorized user to configure or modify the dosage regimen. As an example, an authentication module grants a patient's doctor the ability to configure a dosage regimen upon entry of an authentication code, and subsequent grants the patient the ability to administer a dose based on biometric identification using the patient's fingerprint.

In some embodiments, the drug delivery device monitors delivery of the drug formulation for each cartridge. In some embodiments, the drug delivery device logs each administration of the drug formulation for each cartridge. For example, in some embodiments, logged information includes at least one of cartridge ID (e.g., lot number, serial number, an arbitrary assigned number or ID, or some other identifying information), remaining volume, concentration, time and/or date of infusion, duration of infusion, infusion rate, and administered dose (e.g., volume). In some embodiments, the drug delivery device communicates the logged information to a remote authorized user (e.g., via a server or communication device accessible by the authorized user). In some embodiments, a cartridge provides identifying information detectable by the drug delivery device. In some embodiments, a cartridge provides identifying information via an RFID (radio frequency identification), microchip, barcode, magnetic stripes, or other mechanism for providing identifying information. In some embodiments, a drug delivery device comprises a detector or reader for obtaining identifying information from the cartridge.

In some embodiments, tamper resistant features include tamper evident packaging that indicates unauthorized use or access to the stored drug formulation. For example, in some embodiments, a subject must return or present one or more disposable cartridges when seeking to obtain more cartridges (e.g., refilling or renewing a prescription) at which point a healthcare provider can examine the device and/or cartridge for signs of tampering (e.g., damage or breach). In some embodiments, the prescription refill or renewal is denied when tampering is detected. In some embodiments, the doctor or healthcare provider who gave the prescription is informed of the tampering.

In some embodiments, a drug delivery device monitors attempts to configure or modify the dosage regimen. In some embodiments, the drug delivery device maintains a log of attempts to configure or modify the dosage regimen. In some embodiments, the drug delivery device maintains a log of all changes to the dosage regimen. In some embodiments, the drug delivery device communicates one or more attempts to configure/modify the dosage regimen and/or one or more changes to the dosage regimen over a network to a remote authorized user (e.g., the subject's doctor). In some embodiments, communications to the remote authorized user are stored on a server or network device that is accessible by the remote authorized user (e.g., viewable over the Internet via a web API).

In some embodiments, tamper resistant features include preloaded cartridges to avoid the need for subjects to self-charge the devise with the formulation. In some embodiments, tamper resistant features a rubber membrane of sufficient thickness on preloaded cartridges to prohibit access to the formulation by means other than the access port needle on the accompanying catheter. In some embodiments, tamper resistant features include lockout times to be determined by a user during which the subject cannot select and administer a treatment. In some embodiments, a lockout time is initiated upon detection of an attempt to tamper with the device and/or administer one or more doses outside of the subject's authorized use. For example, repeated attempts to increase the dosage beyond a preset dosage limit may initiate a lockout time. In some embodiments, a lockout time is a period during which device access is locked such that a dose cannot be administered by the subject. In some embodiments, the device is locked out during an ongoing dose (e.g., user is self-administering a continuous infusion dose and repeatedly attempts to increase the dose beyond a dosage limit).

### Treatment of Physical, Psychiatric, or Neurological Disorders

Disclosed herein are systems, devices, kits, formulations, and methods for the treatment of one or more medical and/or psychiatric disorders. In some embodiments, a psychiatric disorder is a major depressive disorder, treatment resistant major depressive disorder, suicidality, suicidal ideation, dysthymia or persistent depressive disorder, bipolar depressive disorder type I, bipolar depressive disorder type II, chronic pain, eating disorder NOS, pain disorder NOS, panic disorder, post-traumatic stress disorder, obsessive-compulsive disorder, complex regional pain syndrome, or reflex sympathetic dystrophy. In some embodiments, the psychiatric disorder being treated is depression, major depressive disorder, or treatment resistant major depression.

In some embodiments, the systems, devices, kits, formulations, and methods disclosed herein are used to administer the drug formulation according to the at least one dosage regimen for treating chronic pain. In some embodiments, the dosage regimen is configured for treating acute pain. In some embodiments, the dosage regimen is configured treating for chronic regional pain syndrome. In some embodiments, the dosage regimen is configured for treating pain associated with Ehlers-Danlos Syndrome. In some embodiments, the dosage regimen is configured for treating post laminectomy syndrome. In some embodiments, the dosage regimen is configured for treating pain associated with post laminectomy syndrome. In some embodiments, the dosage regimen is configured for treating failed back syndrome. In some embodiments, the dosage regimen is configured for treating pain associated with failed back syndrome. In some embodiments, the dosage regimen is configured for treating post-operative pain. In some embodiments, the dosage regimen is configured for treating diabetic neuropathy.

In some embodiments, the systems, devices, kits, formulations, and methods disclosed herein are used to treat one or more personality disorders. Examples of personality disorders include avoidant personality disorder, dependent personality disorder, antisocial personality disorder, histrionic personality disorder, borderline personality disorder, obsessive-compulsive personality disorder, cyclothymic personality disorder, obsessive compulsive disorder, and impulse control disorder (NOS).

In some embodiments, the systems, devices, kits, formulations, and methods disclosed herein are used to treat one or more eating disorders. Examples of eating disorders include anorexia nervosa and bulimia disorder.

In some embodiments, the systems, devices, kits, formulations, and methods disclosed herein are used to treat one or more of major depressive disorder, treatment resistant major depressive disorder, suicidality, suicidal ideation, dysthymia, bipolar disorder (Type I—Depressed), bipolar disorder (Type II—Depressed), post-traumatic stress disorder (PTSD), panic disorder, generalized anxiety disorder, and substance abuse induced mood disorder.

In some embodiments, the systems, devices, kits, formulations, and methods disclosed herein are used to treat a cognitive or neurological disorder or condition such as Huntington's disease, Parkinson's disease, frontotemporal dementia, dementia, Alzheimer's disease, amyotrophic lateral sclerosis, spinal cord trauma, stroke, diffuse traumatic brain injury, HIV-associated dementia, epilepsy, suicidal ideation, Rett syndrome, dyskinesia, dystonia (unspecified), or pseudobulbar affect.

Disclosed herein are systems, devices, and methods for the treatment of one or more medical and/or psychiatric disorders. In some embodiments, a psychiatric disorder is a major depressive disorder, treatment resistant major depressive disorder, suicidality, suicidal ideation, dysthymia or persistent depressive disorder, bipolar depressive disorder type I, bipolar depressive disorder type II, chronic pain, eating disorder NOS, pain disorder NOS, panic disorder, post-traumatic stress disorder, obsessive-compulsive disorder, complex regional pain syndrome, or reflex sympathetic dystrophy. In some embodiments, the psychiatric disorder being treated is depression, major depressive disorder, or treatment resistant major depression.

In some embodiments, a medical or psychiatric disorder is selected from the group consisting of major depressive disorder, treatment resistant major depressive disorder, suicidality, suicidal ideation, dysthymia, bipolar disorder—type I—depressed, bipolar disorder—type II—depressed, post-traumatic stress disorder, impulse control disorder NOS, personality disorder NOS, avoidant personality disorder, dependent personality disorder, antisocial personality disorder, histrionic personality disorder, borderline personality disorder, obsessive-compulsive personality disorder, cyclothymic disorder, obsessive compulsive disorder, eating disorder—NOS, anorexia nervosa, bulimia nervosa, panic disorder, generalized anxiety disorder, substance abuse induce mood disorder, fibromyalgia, chronic fatigue and immunodeficiency syndrome, fibromyalgia syndrome, myalgia, myositis, chronic fatigue unspecified, postviral fatigue syndrome, chronic fatigue syndrome NOS, benign myalgic encephalomyelitis, other fatigue, neoplastic (malignant) related fatigue, other malaise and fatigue, drug dependence—NOS, opiate dependence, benzodiazepine dependence, sedative (hypnotic or anxiolytic dependence), alcohol dependence, stimulant dependence, cocaine dependence, cannabis detoxification, opiate dependence (with withdrawal), benzodiazepine dependence (with withdrawal), sedative (hypnotic or anxiolytic dependence with withdrawal), alcohol dependence (with withdrawal), stimulant dependence (with withdrawal), cocaine dependence (with withdrawal), cannabis detoxification (with withdrawal), pain disorder—not otherwise specified (NOS), pain (unspecified), acute pain, body aches, buttock muscular pain, chronic back pain for greater than 3 months, chronic back pain greater than 3 months duration, chronic coccygeal pain for greater than 3 months, chronic low back pain, chronic low back pain for greater than 3 months, chronic low back pain greater than 3 months duration, chronic malignant pain, chronic neck pain, chronic nonmalignant pain, chronic pain, chronic pain due to malignancy, generalized aches and pains, generalized pain, neck pain (chronic), pain, pain crisis, pain in buttock, pain of coccyx greater than 3 months (chronic), neoplasm related pain (acute) (chronic), other chronic post-procedural pain, chronic pain due to bilateral total hip arthroplasty, chronic pain due to bilateral total knee arthroplasty, chronic pain due to left total hip arthroplasty, chronic pain due to left total knee replacement, chronic pain due to right total hip arthroplasty, chronic pain due to right total knee replacement, chronic pain following bilateral total hip arthroplasty, chronic pain following bilateral total knee arthroplasty, chronic pain following left total hip arthroplasty, chronic pain following left total knee arthroplasty, chronic pain following right total hip arthroplasty, chronic pain following right total knee arthroplasty, chronic pain due to bilateral partial hip arthroplasty, chronic pain due to bilateral partial knee arthroplasty, chronic pain due to left partial hip arthroplasty, chronic pain due to left partial knee replacement, chronic pain due to right partial hip arthroplasty, chronic pain due to right partial knee replacement, chronic pain following bilateral partial hip arthroplasty, chronic pain following bilateral partial knee arthroplasty, chronic pain following left partial hip arthroplasty, chronic pain following left partial knee arthroplasty, chronic pain following right partial hip arthroplasty, chronic pain following right partial knee arthroplasty, acute malignant pain, acute neck pain, acute nonmalignant pain, acute pain, acute pain due to malignancy, generalized aches and pains, generalized pain, neck pain (acute), pain, pain crisis, pain in buttock, pain of coccyx greater than 3 months (acute), neoplasm related pain (acute) (acute), other acute post-procedural pain, acute pain due to bilateral total hip arthroplasty, acute pain due to bilateral total knee arthroplasty, acute pain due to left total hip arthroplasty, acute pain due to left total knee replacement, acute pain due to right total hip arthroplasty, acute pain due to right total knee replacement, acute pain following bilateral total hip arthroplasty, acute pain following bilateral total knee arthroplasty, acute pain following left total hip arthroplasty, acute pain following left total knee arthroplasty, acute pain following right total hip arthroplasty, acute pain following right total knee arthroplasty, acute pain due to bilateral partial hip arthroplasty, acute pain due to bilateral partial knee arthroplasty, acute pain due to left partial hip arthroplasty, acute pain due to left partial knee replacement, acute pain due to right partial hip arthroplasty, acute pain due to right partial knee replacement, acute pain following bilateral partial hip arthroplasty, acute pain following bilateral partial knee arthroplasty, acute pain following left partial hip arthroplasty, acute pain following left partial knee arthroplasty, acute pain following right partial hip arthroplasty, acute pain following right partial knee arthroplasty, pain due to bilateral total hip arthroplasty, pain due to bilateral total knee arthroplasty, pain due to left total hip arthroplasty, pain due to left total knee replacement, pain due to right total hip arthroplasty, pain due to right total knee replacement, pain following bilateral total hip arthroplasty, pain following bilateral total knee arthroplasty, pain following left total hip arthroplasty, pain following left total knee arthroplasty, pain following right total hip arthroplasty, pain following right total knee arthroplasty, pain due to bilateral partial hip arthroplasty, pain due to bilateral partial knee arthroplasty, pain due to left partial hip arthroplasty, pain due to left partial knee replacement, pain due to right partial hip arthroplasty, pain due to right partial knee replacement, pain following bilateral partial hip arthroplasty, pain following bilateral partial knee arthroplasty, pain following left partial hip arthroplasty, pain following left partial knee arthroplasty, pain following right partial hip arthroplasty, pain following right partial knee arthroplasty, acute post-mastectomy pain, acute postoperative pain, acute pain due to trauma or injury, acute pain syndrome, acute pain associated with psychosocial dysfunction, psychosocial dysfunction due to acute pain, neoplasm related pain (acute) (acute), neoplasm related pain, pain due to neoplasm, pain due to neoplastic disease, causalgia (lower limb), causalgia (upper limb), central pain syndrome, acute pain syndrome, complex regional pain syndrome ii (lower limb), complex regional pain syndrome ii (upper limb), phantom limb syndrome with pain, phantom limb syndrome without pain, neoplasm related acute pain, chronic post-mastectomy pain, chronic postoperative pain, chronic pain due to trauma or injury, chronic pain syndrome, chronic pain associated with psychosocial dysfunction, psychosocial dysfunction due to chronic pain, neoplasm related pain (acute) (chronic), neoplasm related pain, pain due to neoplasm, pain due to neoplastic disease, causalgia (lower limb), causalgia (upper limb), central pain syndrome, chronic pain syndrome, complex regional pain syndrome ii (lower limb), complex regional pain syndrome ii (upper limb), phantom limb syndrome with pain, phantom limb syndrome without pain, neoplasm related chronic pain, reflex sympathetic dystrophy, hereditary and idiopathic neuropathy (unspecified), paraneoplastic neuromyopathy and neuropathy (synonyms follow), neuropathy (nerve damage) (paraneoplastic), neuropathy (nerve damage) (peripheral paraneoplastic), paraneoplastic neuropathy, paraneoplastic peripheral neuropathy, type 2 diabetes mellitus with diabetic neuropathy (unspecified), diabetes 2 with neurogenic erectile dysfunction, diabetes type 2 with peripheral neuropathy, diabetes type 2 with peripheral sensory neuropathy, diabetes type 2 with neuropathy, diabetic peripheral neuropathy associated with type 2 diabetes mellitus, diabetes mellitus 2 with neuropathic ulcer foot and heel, neurogenic erectile dysfunction due to type 2 diabetes mellitus, neuropathic midfoot and/or heel ulcer due to type 2 diabetes mellitus, neuropathy due to type 2 diabetes mellitus, peripheral neuropathy due to type 2 diabetes mellitus, peripheral sensory neuropathy due to type 2 diabetes mellitus, other specified diabetes mellitus with diabetic autonomic (poly)neuropathy, other chronic pain, postherpetic polyneuropathy, acute herpes zoster neuropathy, herpes zoster radiculitis, herpes zoster with nervous system complication, herpes zoster with nervous system complications, postherpetic neuralgia, postherpetic radiculopathy, postherpetic myelitis, postherpetic geniculate ganglionitis, postherpetic trigeminal neuralgia, diabetic neuropathy, neuropathy NOS, post-laminectomy syndrome, low back pain, post-surgical pain, endometriosis, migraine, hemiplegic migraine, migraine with aura (intractable), hemiplegic migraine (intractable), other migraine (intractable), migraine, unspecified (intractable), migraine headache NOS, migraine without aura (intractable), hemiplegic migraine (not intractable), other migraine (not intractable), migraine, unspecified (not intractable), hemiplegic migraine (intractable, without status migrainosus), migraine with aura, ophthalmoplegic migraine (not intractable), abdominal migraine (not intractable), migraine with aura (not intractable, with status migrainosus), migraine (unspecified, intractable, with status migrainosus), migraine without aura, migraine without aura (not intractable), migraine with aura (not intractable), chronic migraine without aura, migraine (unspecified, not intractable, without status migrainosus), migraine (unspecified, intractable without status migrainosus), other migraine (intractable, without status migrainosus), abdominal migraine (intractable; intractable allergic migraine; intractable ophthalmic migraine), other migraine (not intractable, without status migrainosus), menstrual migraine (intractable, without status migrainosus), Huntington's disease, Parkinson's disease, frontotemporal dementia, dementia, Alzheimer's disease, amyotrophic lateral sclerosis, spinal cord trauma, stroke, diffuse traumatic brain injury, hiv-associated dementia, epilepsy, suicidal ideation, Rett syndrome, dyskinesia, dystonia (unspecified), pseudobulbar affect, tinnitus (unspecified ear), glaucoma.

In some embodiments, the systems, devices, kits, formulations, and methods disclosed herein are used to treat one or more fatigue and fatigue-related disorders. Examples of fatigue and fatigue-related disorders include fibromyalgia, fibromyalgia syndrome, chronic fatigue and immunodeficiency syndrome, myalgia, myositis, chronic fatigue (unspecified), postviral fatigue syndrome, chronic fatigue syndrome (NOS), benign myalgic encephalomyelitis, neoplastic (malignant) related fatigue, and other malaise and fatigue.

In some embodiments, the systems, devices, kits, formulations, and methods disclosed herein are used to treat post-laminectomy syndrome (e.g., failed back syndrome). In some embodiments, the systems, devices, kits, formulations, and methods disclosed herein are used to treat post-operative pain. In some embodiments, the systems, devices, kits, formulations, and methods disclosed herein are used to treat cancer pain. In some embodiments, the systems, devices, kits, formulations, and methods disclosed herein are used to treat osteoarthritis. In some embodiments, the systems, devices, kits, formulations, and methods disclosed herein are used to treat fibromyalgia.

In some embodiments, the systems, devices, kits, formulations, and methods disclosed herein are used to treat pain or a pain disorder. In some embodiments, chronic pain refers to pain having a duration of greater than 3 months. Examples of pain and pain disorders include pain that is not otherwise specified (NOS) such as acute pain, body aches, buttock muscular pain, lower back pain, chronic back pain, chronic coccygeal pain, chronic low back pain, chronic malignant pain, chronic neck pain, chronic nonmalignant pain, chronic pain, and generalized pain. In some embodiments, the pain can include pain crisis, pain in buttocks, pain of coccyx (chronic or acute), or neoplasm related pain (chronic or acute).

In some embodiments, the pain is chronic post-procedural and/or post-surgical pain. Examples of post-procedural pain include chronic pain due to bilateral total hip arthroplasty, chronic pain due to bilateral total knee arthroplasty, chronic pain due to left total hip arthroplasty, chronic pain due to left total knee replacement, chronic pain due to right total hip arthroplasty, chronic pain due to right total knee replacement, chronic pain following bilateral partial hip arthroplasty, chronic pain following bilateral partial knee arthroplasty, chronic pain following left partial hip arthroplasty, chronic pain following left partial knee arthroplasty, chronic pain following right partial hip arthroplasty, chronic pain following right partial knee arthroplasty, pain due to bilateral total hip arthroplasty, pain due to bilateral total knee arthroplasty, pain due to left total hip arthroplasty, pain due to left total knee replacement, pain due to right total hip arthroplasty, pain due to right total knee replacement, pain following bilateral partial hip arthroplasty, pain following bilateral partial knee arthroplasty, pain following left partial hip arthroplasty, pain following left partial knee arthroplasty, pain following right partial hip arthroplasty, pain following right partial knee arthroplasty, chronic post-mastectomy pain, chronic post-mastectomy pain, and chronic postoperative pain.

In some embodiments, the pain is chronic pain due to trauma or injury. In some embodiments, the pain is a chronic pain syndrome, also referred to as chronic pain associated with psychosocial dysfunction or psychosocial dysfunction due to chronic pain. In some embodiments, the pain is a neoplasm related pain or pain due to neoplastic disease (chronic or acute). In some embodiments, the pain is causalgia (lower limb and/or upper limb).

In some embodiments, the pain is central pain syndrome, complex regional pain syndrome I, complex regional pain syndrome II (lower limb), or complex regional pain syndrome II (upper limb).

In some embodiments, the systems, devices, kits, formulations, and methods disclosed herein are used to treat disorders such as fibromyalgia, fibromyalgia syndrome, chronic fatigue and immunodeficiency syndrome, myalgia, myositis, chronic fatigue (unspecified), postviral fatigue syndrome, chronic fatigue syndrome (NOS), benign myalgic encephalomyelitis, phantom limb syndrome (with or without pain), reflex sympathetic dystrophy, hereditary and idiopathic neuropathy, and paraneoplastic neuromyopathy and neuropathy (including peripheral neuropathy), type 2 diabetes mellitus (with diabetic neuropathy, unspecified), or specified diabetes mellitus with diabetic autonomic (poly)neuropathy. Examples of type 2 diabetes mellitus with unspecified diabetic neuropathy include diabetes with neurogenic erectile dysfunction, peripheral neuropathy, peripheral sensory neuropathy, neuropathy, and neuropathic ulcer (e.g. foot and heel).

In some embodiments, the systems, devices, kits, formulations, and methods disclosed herein are used to treat disorders such as postherpetic polyneuropathy, acute herpes zoster neuropathy, herpes zoster radiculitis, herpes zoster with nervous system complication, herpes zoster with nervous system complications, postherpetic neuralgia, postherpetic radiculopathy, postherpetic myelitis, postherpetic geniculate ganglionitis, or postherpetic trigeminal neuralgia.

In some embodiments, the systems, devices, kits, formulations, and methods disclosed herein are used to treat disorders or conditions such as post-laminectomy syndrome and endometriosis (unspecified). In some embodiments, the systems, devices, kits, formulations, and methods disclosed herein are used to treat migraines such as migraine with aura (intractable), migraine with aura (not intractable), hemiplegic migraine (intractable), migraine (unspecified, intractable), migraine headache (NOS), migraine without aura (intractable), migraine without aura (not intractable), migraine (unspecified, not intractable), hemiplegic migraine (intractable, without status migrainosus), other migraine (intractable), ophthalmoplegic migraine (not intractable), abdominal migraine (not intractable), abdominal migraine (intractable), intractable allergic migraine, intractable ophthalmic migraine, migraine with aura (not intractable, with status migrainosus), migraine (unspecified, not intractable, with status migrainosus), migraine (unspecified, intractable, with status migrainosus), chronic migraine without aura, migraine (unspecified, not intractable, without status migrainosus), migraine (unspecified, intractable, without status migrainosus), menstrual migraine (intractable, without status migrainosus), other migraine (intractable, without status migrainosus), and other migraine (not intractable, without status migrainosus).

In some embodiments, the systems, devices, kits, formulations, and methods disclosed herein are used to treat a disease or condition such as tinnitus (unspecified ear) or glaucoma.

In some embodiments, the systems, devices, kits, formulations, and methods disclosed herein are used to treat any combination of disorders or conditions described herein.

### Treatment of Drug Dependence

In some embodiments, the systems, devices, kits, formulations, and methods disclosed herein are used to treat drug dependence. Examples of drug dependence include opiate dependence, benzodiazepine dependence, sedative (hypnotic or anxiolytic) dependence, alcohol dependence, stimulant dependence, cocaine dependence, cannabis detoxification, opiate dependence (with withdrawal), benzodiazepine dependence (with withdrawal), sedative (with withdrawal) dependence, alcohol dependence (with withdrawal), stimulant dependence (with withdrawal), cocaine dependence (with withdrawal), and cannabis detoxification (with withdrawal).

### Digital Processing Device

In some embodiments, the platforms, media, methods and applications described herein include a digital processing device 101, a processor 105, or use of the same. In further embodiments, the digital processing device 101 includes one or more hardware central processing units (CPU) 105 that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage 115 and/or memory 110 device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In some embodiments, the non-volatile memory comprises magnetoresistive random-access memory (MRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a subject. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In some embodiments, the display is E-paper or E ink. In other embodiments, the display is a video projector. In still further embodiments, the display is a combination of devices such as those disclosed herein.

In some embodiments, the digital processing device includes an input device to receive information from a subject. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, media, methods and applications described herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, media, methods and applications described herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash °, HTML 5, Apple® QuickTime®, Microsoft Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. In addition, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the platforms, media, methods and applications described herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of barcode, route, parcel, subject, or network information. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

### Web Browser Plug-in

In some embodiments, the computer program includes a web browser plug-in. In computing, a plug-in is one or more software components that add specific functionality to a larger software application. Makers of software applications support plug-ins to enable third-party developers to create abilities that extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon Kindle Basic Web, Nokia Browser, Opera Software Opera Mobile, and Sony® PSP™ browser.

### Numbered Embodiments

The following embodiments recite nonlimiting permutations of combinations of features disclosed herein. Other permutations of combinations of features are also contemplated. In particular, each of these numbered embodiments is contemplated as depending from or relating to every previous or subsequent numbered embodiment, independent of their order as listed.

1. A drug delivery device comprising: a) a pump mechanism configured for administering a drug formulation comprising an NMDA receptor antagonist; and b) a user interface allowing a subject to select and self-administer a dose of the drug formulation from a selection of at least one pre-programmed dosage regimen that is not configurable by the subject; wherein the at least one dosage regimen provides an effective drug plasma concentration. 2. The drug delivery device of embodiment 1, wherein the at least one dosage regimen provides an effective steady state drug plasma concentration. 3. The drug delivery device of embodiment 1, wherein the at least one dosage regimen is locked after configuration by an authorized user to deter modification by the subject. 4. The drug delivery device of embodiment 1, wherein the at least one dosage regimen is locked after configuration by the manufacturer to deter modification by the patient. 5. The drug delivery device of embodiment 1, wherein the drug delivery device is configured to be tamper-resistant to deter administration of a dose of the drug formulation that deviates from the at least one dosage regimen. 6. The drug delivery device of embodiment 1, wherein the drug formulation is stored in tamper-resistant cartridge. 7. The drug delivery device of embodiment 1, wherein the drug delivery device comprises a reservoir for storing the drug formulation prior to administration. 8. The drug delivery device of embodiment 1, wherein the at least one dosage regimen reduces side effects of the drug formulation while providing the state drug plasma concentration. 9. The drug delivery device of embodiment 8, wherein the side effects comprise drug dependence or addiction. 10. The drug delivery device of embodiment 8, wherein the side effects comprise hallucination, disorientation, dissociation, dizziness, drowsiness, increased heart rate, elevated blood pressure, nausea, vomiting, fatigue, brain fog, confusion, anxiety, distress, shortness of breath, or any combination thereof. 11. The drug delivery device of embodiment 1, wherein the drug delivery device deters abuse of the drug formulation by limiting control of the at least one dosage regimen by the subject. 12. The drug delivery device of embodiment 1, wherein the drug delivery device is configured to administer the drug formulation according to the at least one dosage regimen for treating Treatment Resistant Depression. 13. The drug delivery device of embodiment 1, wherein the drug delivery device is configured to administer the drug formulation according to the at least one dosage regimen for treating major depressive disorder, treatment resistant major depressive disorder, suicidality, suicidal ideation, dysthymia or persistent depressive disorder, bipolar depressive disorder type I, bipolar depressive disorder type II, chronic pain, eating disorder NOS, pain disorder NOS, panic disorder, post-traumatic stress disorder, obsessive-compulsive disorder, complex regional pain syndrome, reflex sympathetic dystrophy, or any combination thereof. 14. The drug delivery device of embodiment 1, wherein the NMDA receptor antagonist is ketamine or a pharmaceutically acceptable salt thereof. 15. The drug delivery device of embodiment 1, wherein the NMDA receptor antagonist is an arylcyclohexylamine or arylcyclohexylamine derivative. 16. The drug delivery device of embodiment 1, wherein the NMDA receptor antagonist also acts as a dopamine reuptake inhibitor, μ-opioid receptor agonist, σ receptor agonist, nACh receptor antagonist, D2 receptor agonistic, or any combination thereof 17. The drug delivery device of embodiment 1, wherein the NMDA receptor antagonist is ketamine, phencyclidine (PCP), 3-MeO-Phencylidine, 4-MeO-Phencyclidine, eticyclidine (PCE), 3-MeO-PCE, methoxetamine (MXE), tiletamine, or tenocyclidine (TCP). 18. The drug delivery device of embodiment 1, wherein the drug formulation comprises a second active ingredient for mitigating side effects of the NMDA receptor antagonist. 19. The drug delivery device of embodiment 18, wherein the second active ingredient is a benzodiazepine, a selective serotonin 5-HT3 receptor antagonist, or a beta blocker. 20. The drug delivery device of embodiment 1, wherein the drug formulation comprises a second active ingredient for altering pharmacokinetic properties of the NMDA receptor antagonist. 21. The drug delivery device of embodiment 20, wherein the second active ingredient is an inhibitor of CYP2B6 and/or CYP3A and/or CYP2C9. 22. The drug delivery device of embodiment 1, wherein the at least one dosage regimen is configured by an authorized user who is a healthcare provider for the subject. 23. The drug delivery device of embodiment 1, wherein the at least one dosage regimen is prescribed for the subject by a healthcare provider. 24. The drug delivery device of embodiment 1, wherein the subject is not authorized to configure or modify the at least one dosage regimen. 25. The drug delivery device of embodiment 1, wherein the drug delivery device allows limited modification of the at least one dosage regimen by the subject. 26. The drug delivery device of embodiment 1, wherein the at least one dosage regimen comprises a plurality of dosing options selectable by the subject. 27. The drug delivery device of embodiment 26, wherein the plurality of dosing options is selected from the group consisting of bolus injection, continuous infusion. 28. The drug delivery device of embodiment 26, wherein the plurality of dosing options comprises differences in dosage size, dosage rate, infusion duration, or any combination thereof. 29. The drug delivery device of embodiment 1, further comprising a remote access module allowing an authorized user to remotely configure or modify the at least one dosage regimen over a network. 30. The drug delivery device of embodiment 1, further comprising a data module storing information for doses administered by the subject. 31. The drug delivery device of embodiment 1, further comprising a monitoring module allowing an authorized user to remotely monitor the at least one dosage regimen over a network. 32. The drug delivery device of embodiment 1, further comprising a communications module allowing the subject to send a request to an authorized user regarding the at least one dosage regimen over a network. 33. The drug delivery device of embodiment 1, further comprising a communications module allowing the drug delivery device to send and receive information over a network. 34. The drug delivery device of embodiment 1, further comprising a communications module allowing the drug delivery device to pair with a communications device that provides a network connection for communicating with an authorized user. 35. The drug delivery device of embodiment 1, wherein the at least one dosage regimen comprises a dosage limit setting an upper limit on a size of the dose. 36. The drug delivery device of embodiment 1, wherein the drug delivery device prohibits administration of a dose of the drug formulation that exceeds a dosage limit. 37. The drug delivery device of embodiment 1, wherein the drug delivery device prohibits administration of a dose of the drug formulation that causes a total daily dose to exceed a daily dosage limit. 38. The drug delivery device of embodiment 1, wherein the drug delivery device prohibits administration of a dose of the drug formulation at an infusion rate that exceeds a dosage limit. 39. The drug delivery device of embodiment 1, wherein the drug delivery device deters abuse of the drug formulation. 40. The drug delivery device of embodiment 1, wherein the pump mechanism is configured to administer the drug formulation through subcutaneous or intramuscular injection. 41. The drug delivery device of embodiment 1, wherein the dose comprises an infusion rate of at least about 0.1 mg/hour. 42. The drug delivery device of embodiment 1, wherein the dose comprises an infusion rate of no more than about 200 mg/hour. 43. The drug delivery device of embodiment 1, wherein the dose comprises an infusion rate from about 0.1 mg/hour to about 200 mg/hour. 44. The drug delivery device of embodiment 1, wherein the dose comprises an infusion of at least about ten (10) minutes. 45. The drug delivery device of embodiment 1, wherein the dose comprises an infusion that is continuous. 46. The drug delivery device of embodiment 1, wherein the dose comprises an infusion rate of at least 1 mg/hour for at least ten (10) minutes. 47. The drug delivery device of embodiment 1, wherein the NMDA receptor antagonist is a racemic mixture of ketamine. 48. The drug delivery device of embodiment 1, wherein the NMDA receptor antagonist is substantially pure S-ketamine. 49. The drug delivery device of embodiment 1, wherein the NMDA receptor antagonist is substantially pure R-ketamine. 50. The drug delivery device of embodiment 1, wherein the dosage regimen provides a clinically effective steady-state concentration of the NMDA receptor antagonist outside of a hospital or clinical setting. 51. The drug delivery device of embodiment 1, wherein the dosage regimen provides a clinically effective steady-state concentration of the NMDA receptor antagonist for at least 1 week. 52. The drug delivery device of embodiment 1, wherein the dosage regimen provides an average treatment steady state plasma concentration of at least 1 ng/mL with a peak trough fluctuation of no more than 100% of the average steady state plasma concentration during treatment. 53. The drug delivery device of embodiment 1, wherein the at least one dosage regimen provides a clinically effective steady-state concentration of the NMDA receptor antagonist with peak trough fluctuation of no more than 100% while the steady-state plasma concentration is maintained. 54. The drug delivery device of embodiment 1, wherein the at least one dosage regimen provides a clinically effective steady-state concentration of the NMDA receptor antagonist with a Cmax to Cmin ratio of no more than 4. 55. The drug delivery device of embodiment 1, wherein the at least one dosage regimen provides a concentration of the NMDA receptor antagonist of at least 1 ng/mL throughout a duration of the at least one dosage regimen. 56. The drug delivery device of embodiment 1, wherein the at least one dosage regimen comprises at least 1 dose per month. 57. The drug delivery device of embodiment 1, wherein the at least one dosage regimen comprises a single continuous dose. 58. The drug delivery device of embodiment 1, wherein the at least one dosage regimen comprises a loading dose and a series of maintenance doses. 59. The drug delivery device of embodiment 1, wherein the at least one dosage regimen comprises periodic doses. 60. The drug delivery device of embodiment 1, wherein the at least one dosage regimen comprises aperiodic doses. 61. A system comprising: a) a drug delivery device comprising a pump mechanism for administering a drug formulation comprising an NMDA receptor antagonist and a user interface allowing a subject to self-administer a dose of the drug formulation from a selection of at least one pre-programmed dosage regimen that is not configurable by the subject; and b) a digital device of an authorized user in communication with the drug delivery device to allow the authorized user to configure, modify, or monitor the dosage regimen; wherein the at least one dosage regimen provides an effective steady state drug plasma concentration while reducing side effects. 62. The system of embodiment 61, wherein the at least one dosage regimen provides an effective steady state drug plasma concentration. 63. The system of embodiment 61, wherein the at least one dosage regimen is locked after configuration by an authorized user to deter modification by the subject. 64. The system of embodiment 61, wherein the at least one dosage regimen is locked after configuration by the manufacturer to deter modification by the patient. 65. The system of embodiment 61, wherein the drug delivery device is configured to be tamper-resistant to deter administration of a dose of the drug formulation that deviates from the at least one dosage regimen. 66. The system of embodiment 61, wherein the drug formulation is stored in tamper-resistant cartridge. 67. The system of embodiment 61, wherein the drug delivery device comprises a reservoir for storing the drug formulation prior to administration. 68. The system of embodiment 61, wherein the at least one dosage regimen reduces side effects of the drug formulation while providing the state drug plasma concentration. 69. The system of embodiment 68, wherein the side effects comprise drug dependence or addiction. 70. The system of embodiment 68, wherein the side effects comprise hallucination, disorientation, dissociation, dizziness, drowsiness, increased heart rate, elevated blood pressure, nausea, vomiting, fatigue, brain fog, confusion, anxiety, distress, shortness of breath or any combination thereof. 71. The system of embodiment 61, wherein the drug delivery device deters abuse of the drug formulation by limiting control of the at least one dosage regimen by the subject. 72. The system of embodiment 61, wherein the drug delivery device is configured to administer the drug formulation according to the at least one dosage regimen for treating Treatment Resistant Depression. 73. The system of embodiment 61, wherein the drug delivery device is configured to administer the drug formulation according to the at least one dosage regimen for treating major depressive disorder, treatment resistant major depressive disorder, suicidality, suicidal ideation, dysthymia or persistent depressive disorder, bipolar depressive disorder type I, bipolar depressive disorder type II, chronic pain, eating disorder NOS, pain disorder NOS, panic disorder, post-traumatic stress disorder, obsessive-compulsive disorder, complex regional pain syndrome, reflex sympathetic dystrophy, or any combination thereof. 74. The system of embodiment 61, wherein the NMDA receptor antagonist is ketamine or a pharmaceutically acceptable salt thereof. 75. The system of embodiment 61, wherein the NMDA receptor antagonist is an arylcyclohexylamine or arylcyclohexylamine derivative. 76. The system of embodiment 61, wherein the NMDA receptor antagonist also acts as a dopamine reuptake inhibitor, μ-opioid receptor agonist, σ receptor agonist, nACh receptor antagonist, D2 receptor agonistic, or any combination thereof. 77. The system of embodiment 61, wherein the NMDA receptor antagonist is ketamine, phencyclidine (PCP), 3-MeO-Phencylidine, 4-MeO-Phencyclidine, eticyclidine (PCE), 3-MeO-PCE, methoxetamine (MXE), tiletamine, or tenocyclidine (TCP). 78. The system of embodiment 61, wherein the drug formulation comprises a second active ingredient for mitigating side effects of the NMDA receptor antagonist. 79. The system of embodiment 78, wherein the second active ingredient is a benzodiazepine, a selective serotonin 5-HT3 receptor antagonist, or a beta blocker. 80. The system of embodiment 61, wherein the drug formulation comprises a second active ingredient for altering pharmacokinetic properties of the NMDA receptor antagonist. 81. The system of embodiment 80, wherein the second active ingredient is an inhibitor of CYP2B6 and/or CYP3A and/or CYP2C9. 82. The system of embodiment 61, wherein the at least one dosage regimen is configured by an authorized user who is a healthcare provider for the subject. 83. The system of embodiment 61, wherein the at least one dosage regimen is prescribed for the subject by a healthcare provider. 84. The system of embodiment 61, wherein the subject is not authorized to configure or modify the at least one dosage regimen. 85. The system of embodiment 61, wherein the drug delivery device allows limited modification of the at least one dosage regimen by the subject. 86. The system of embodiment 61, wherein the at least one dosage regimen comprises a plurality of dosing options selectable by the subject. 87. The system of embodiment 86, wherein the plurality of dosing options is selected from the group consisting of bolus injection, continuous infusion. 88. The system of embodiment 86, wherein the plurality of dosing options comprises differences in dosage size, dosage rate, infusion duration, or any combination thereof 89. The system of embodiment 61, further comprising a remote access module allowing an authorized user to remotely configure or modify the at least one dosage regimen over a network. 90. The system of embodiment 61, further comprising a data module storing information for doses administered by the subject. 91. The system of embodiment 61, further comprising a monitoring module allowing an authorized user to remotely monitor the at least one dosage regimen over a network. 92. The system of embodiment 61, further comprising a communications module allowing the subject to send a request to an authorized user regarding the at least one dosage regimen over a network. 93. The system of embodiment 61, further comprising a communications module allowing the drug delivery device to send and receive information over a network. 94. The system of embodiment 61, further comprising a communications module allowing the drug delivery device to pair with a communications device that provides a network connection for communicating with an authorized user. 95. The system of embodiment 61, wherein the at least one dosage regimen comprises a dosage limit setting an upper limit on a size of the dose. 96. The system of embodiment 61, wherein the drug delivery device prohibits administration of a dose of the drug formulation that exceeds a dosage limit. 97. The system of embodiment 61, wherein the drug delivery device prohibits administration of a dose of the drug formulation that causes a total daily dose to exceed a daily dosage limit. 98. The system of embodiment 61, wherein the drug delivery device prohibits administration of a dose of the drug formulation at an infusion rate that exceeds a dosage limit. 99. The system of embodiment 61, wherein the drug delivery device deters abuse of the drug formulation. 100. The system of embodiment 61, wherein the pump mechanism is configured to administer the drug formulation through subcutaneous or intramuscular injection. 101. The system of embodiment 61, wherein the dose comprises an infusion rate of at least about 1 mg/hour. 102. The system of embodiment 61, wherein the dose comprises an infusion rate of no more than about 200 mg/hour. 103. The system of embodiment 61, wherein the dose comprises an infusion rate from about 1 mg/hour to about 200 mg/hour. 104. The system of embodiment 61, wherein the dose comprises an infusion of at least about ten (10) minutes. 105. The system of embodiment 61, wherein the dose comprises an infusion that is continuous. 106. The system of embodiment 61, wherein the dose comprises an infusion rate of at least 1 mg/hour for at least ten (10) minutes. 107. The system of embodiment 61, wherein the NMDA receptor antagonist is a racemic mixture of ketamine. 108. The system of embodiment 61, wherein the NMDA receptor antagonist is substantially pure S-ketamine. 109. The system of embodiment 61, wherein the NMDA receptor antagonist is substantially pure R-ketamine. 110. The system of embodiment 61, wherein the dosage regimen provides a clinically effective steady-state concentration of the NMDA receptor antagonist outside of a hospital or clinical setting. 111. The system of embodiment 61, wherein the dosage regimen provides a clinically effective steady-state concentration of the NMDA receptor antagonist for at least 1 week. 112. The system of embodiment 61, wherein the dosage regimen provides an average treatment steady state plasma concentration of at least 1 ng/mL with a peak trough fluctuation of no more than 100% of the average steady state plasma concentration during treatment. 113. The system of embodiment 61, wherein the at least one dosage regimen provides a clinically effective steady-state concentration of the NMDA receptor antagonist with peak trough fluctuation of no more than 100% while the steady-state plasma concentration is maintained. 114. The system of embodiment 61, wherein the at least one dosage regimen provides a clinically effective steady-state concentration of the NMDA receptor antagonist with a Cmax to Cmin ratio of no more than 4. 115. The system of embodiment 61, wherein the at least one dosage regimen provides a concentration of the NMDA receptor antagonist of at least 1 ng/mL throughout a duration of the at least one dosage regimen. 116. The system of embodiment 61, wherein the at least one dosage regimen comprises at least 1 dose per month. 117. The system of embodiment 61, wherein the at least one dosage regimen comprises a single continuous dose. 118. The system of embodiment 61, wherein the at least one dosage regimen comprises a loading dose and a series of maintenance doses. 119. The system of embodiment 61, wherein the at least one dosage regimen comprises periodic doses. 120. The system of embodiment 61, wherein the at least one dosage regimen comprises aperiodic doses. 121. A method for self-treatment by a subject outside of a hospital or clinical setting, comprising: a) obtaining a drug delivery device for administering a dose of a drug formulation comprising an NMDA receptor antagonist; and b) self-administering the dose from a selection of at least one pre-programmed dosage regimen that is not configurable by the subject; wherein the at least one dosage regimen provides an effective steady state drug plasma concentration while reducing side effects. 122. The method of embodiment 121, wherein the at least one dosage regimen provides an effective steady state drug plasma concentration. 123. The method of embodiment 121, wherein the at least one dosage regimen is locked after configuration by an authorized user to deter modification by the subject. 124. The method of embodiment 121, wherein the at least one dosage regimen is locked after configuration by the manufacturer to deter modification by the patient. 125. The method of embodiment 121, wherein the drug delivery device is configured to be tamper-resistant to deter administration of a dose of the drug formulation that deviates from the at least one dosage regimen. 126. The method of embodiment 121, wherein the drug formulation is stored in tamper-resistant cartridge. 127. The method of embodiment 121, wherein the drug delivery device comprises a reservoir for storing the drug formulation prior to administration. 128. The method of embodiment 121, wherein the at least one dosage regimen reduces side effects of the drug formulation while providing the state drug plasma concentration. 129. The method of embodiment 128, wherein the side effects comprise drug dependence or addiction. 130. The method of embodiment 128, wherein the side effects comprise hallucination, disorientation, dissociation, dizziness, drowsiness, increased heart rate, elevated blood pressure, nausea, vomiting, fatigue, brain fog, confusion, anxiety, distress, shortness of breath or any combination thereof 131. The method of embodiment 121, wherein the drug delivery device deters abuse of the drug formulation by limiting control of the at least one dosage regimen by the subject. 132. The method of embodiment 121, wherein the drug delivery device is configured to administer the drug formulation according to the at least one dosage regimen for treating Treatment Resistant Depression. 133. The method of embodiment 121, wherein the drug delivery device is configured to administer the drug formulation according to the at least one dosage regimen for treating major depressive disorder, treatment resistant major depressive disorder, suicidality, suicidal ideation, dysthymia or persistent depressive disorder, bipolar depressive disorder type I, bipolar depressive disorder type II, chronic pain, eating disorder NOS, pain disorder NOS, panic disorder, post-traumatic stress disorder, obsessive-compulsive disorder, complex regional pain syndrome, reflex sympathetic dystrophy, or any combination thereof 134. The method of embodiment 121, wherein the NMDA receptor antagonist is ketamine or a pharmaceutically acceptable salt thereof. 135. The method of embodiment 121, wherein the NMDA receptor antagonist is an arylcyclohexylamine or arylcyclohexylamine derivative. 136. The method of embodiment 121, wherein the NMDA receptor antagonist also acts as a dopamine reuptake inhibitor, μ-opioid receptor agonist, σ receptor agonist, nACh receptor antagonist, D2 receptor agonistic, or any combination thereof. 137. The method of embodiment 121, wherein the NMDA receptor antagonist is ketamine, phencyclidine (PCP), 3-MeO-Phencylidine, 4-MeO-Phencyclidine, eticyclidine (PCE), 3-MeO-PCE, methoxetamine (MXE), tiletamine, or tenocyclidine (TCP). 138. The method of embodiment 121, wherein the drug formulation comprises a second active ingredient for mitigating side effects of the NMDA receptor antagonist. 139. The method of embodiment 138, wherein the second active ingredient is a benzodiazepine, a selective serotonin 5-HT3 receptor antagonist, or a beta blocker. 140. The method of embodiment 121, wherein the drug formulation comprises a second active ingredient for altering pharmacokinetic properties of the NMDA receptor antagonist. 141. The method of embodiment 140, wherein the second active ingredient is an inhibitor of CYP2B6 and/or CYP3A and/or CYP2C9. 142. The method of embodiment 121, wherein the at least one dosage regimen is configured by an authorized user who is a healthcare provider for the subject. 143. The method of embodiment 121, wherein the at least one dosage regimen is prescribed for the subject by a healthcare provider. 144. The method of embodiment 121, wherein the subject is not authorized to configure or modify the at least one dosage regimen. 145. The method of embodiment 121, wherein the drug delivery device allows limited modification of the at least one dosage regimen by the subject. 146. The method of embodiment 121, wherein the at least one dosage regimen comprises a plurality of dosing options selectable by the subject. 147. The method of embodiment 146, wherein the plurality of dosing options is selected from the group consisting of bolus injection, continuous infusion. 148. The method of embodiment 146, wherein the plurality of dosing options comprises differences in dosage size, dosage rate, infusion duration, or any combination thereof 149. The method of embodiment 121, further comprising allowing an authorized user to remotely configure or modify the at least one dosage regimen over a network. 150. The method of embodiment 121, further comprising storing information for doses administered by the subject. 151. The method of embodiment 121, further comprising allowing an authorized user to remotely monitor the at least one dosage regimen over a network. 152. The method of embodiment 121, further comprising sending, by the drug delivery device, a request to an authorized user regarding the at least one dosage regimen over a network. 153. The method of embodiment 121, further comprising sending and receiving, by the drug delivery device, information over a network. 154. The method of embodiment 121, further comprising pairing the drug delivery device with a communications device that provides a network connection for communicating with an authorized user. 155. The method of embodiment 121, wherein the at least one dosage regimen comprises a dosage limit setting an upper limit on a size of the dose. 156. The method of embodiment 121, wherein the drug delivery device prohibits administration of a dose of the drug formulation that exceeds a dosage limit. 157. The method of embodiment 121, wherein the drug delivery device prohibits administration of a dose of the drug formulation that causes a total daily dose to exceed a daily dosage limit. 158. The method of embodiment 121, wherein the drug delivery device prohibits administration of a dose of the drug formulation at an infusion rate that exceeds a dosage limit. 159. The method of embodiment 121, wherein the drug delivery device deters abuse of the drug formulation. 160. The method of embodiment 121, wherein the pump mechanism is configured to administer the drug formulation through subcutaneous or intramuscular injection. 161. The method of embodiment 121, wherein the dose comprises an infusion rate of at least about 1 mg/hour. 162. The method of embodiment 121, wherein the dose comprises an infusion rate of no more than about 200 mg/hour. 163. The method of embodiment 121, wherein the dose comprises an infusion rate from about 1 mg/hour to about 200 mg/hour. 164. The method of embodiment 121, wherein the dose comprises an infusion of at least about ten (10) minutes. 165. The method of embodiment 121, wherein the dose comprises an infusion that is continuous. 166. The method of embodiment 121, wherein the dose comprises an infusion rate of at least 1 mg/hour for at least ten (10) minutes. 167. The method of embodiment 121, wherein the NMDA receptor antagonist is a racemic mixture of ketamine. 168. The method of embodiment 121, wherein the NMDA receptor antagonist is substantially pure S-ketamine. 169. The method of embodiment 121, wherein the NMDA receptor antagonist is substantially pure R-ketamine. 170. The method of embodiment 121, wherein the dosage regimen provides a clinically effective steady-state concentration of the NMDA receptor antagonist outside of a hospital or clinical setting. 171. The method of embodiment 121, wherein the dosage regimen provides a clinically effective steady-state concentration of the NMDA receptor antagonist for at least 1 week. 172. The method of embodiment 121, wherein the dosage regimen provides an average treatment steady state plasma concentration of at least 1 ng/mL with a peak trough fluctuation of no more than 100% of the average steady state plasma concentration during treatment. 173. The method of embodiment 121, wherein the at least one dosage regimen provides a clinically effective steady-state concentration of the NMDA receptor antagonist with peak trough fluctuation of no more than 100% while the steady-state plasma concentration is maintained. 174. The method of embodiment 121, wherein the at least one dosage regimen provides a clinically effective steady-state concentration of the NMDA receptor antagonist with a Cmax to Cmin ratio of no more than 4. 175. The method of embodiment 121, wherein the at least one dosage regimen provides a concentration of the NMDA receptor antagonist of at least 1 ng/mL throughout a duration of the at least one dosage regimen. 176. The method of embodiment 121, wherein the at least one dosage regimen comprises at least 1 dose per month. 177. The method of embodiment 121, wherein the at least one dosage regimen comprises a single continuous dose. 178. The method of embodiment 121, wherein the at least one dosage regimen comprises a loading dose and a series of maintenance doses. 179. The method of embodiment 121, wherein the at least one dosage regimen comprises periodic doses. 180. The method of embodiment 121, wherein the at least one dosage regimen comprises aperiodic doses. 181. The method of any of embodiments 121-180, wherein the method is used for treating, preventing, or ameliorating at least one symptom of a disorder, disease, or condition. 182. The method of embodiment 181, wherein the disorder, disease, or condition is a mental or psychiatric disorder, a neurological condition or disorder, pain, or an inflammatory disorder. 183. The method of embodiment 181, wherein the disorder, disease, or condition is pain. 184. The method of embodiment 181, wherein the neurological condition or disorder is chronic pain. 185. The method of embodiment 181, wherein the disorder, disease, or condition is a mental or psychiatric disorder. 186. The method of embodiment 181, wherein the mental or psychiatric disorder is Major Depressive Disorder, treatment resistant major depressive disorder, suicidality, suicidal ideation, Substance-Related Disorder, Sedative-, Hypnotic-, or Anxiolytic-Related Disorder, Sedative-, hypnotic-, or anxiolytic withdrawal, alcohol withdrawal, cannabis dependence, cannabis withdrawal, barbiturate dependence, barbiturate withdrawal, benzodiazepine dependence, benzodiazepine withdrawal, amphetamine dependence, amphetamine withdrawal, opioid dependence, opioid withdrawal, opioid-related disorder, alcohol dependence, cocaine dependence, or cocaine withdrawal. 187. A method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is not configurable by the subject; wherein the dosage regimen provides an average ketamine plasma concentration of at least 1 ng/mL with a peak trough fluctuation of no more than 100%. 188. A drug delivery device comprising: a) a reservoir for storing a drug formulation comprising ketamine; b) an infusion pump connected to the reservoir and configured for subcutaneous infusion of the drug formulation; and c) a user interface enabling a subject to self-administer a dose of the drug formulation according to a pre-programmed dosage regimen that is configurable only by an authorized user; wherein the dosage regimen provides an average ketamine plasma concentration of at least 1 ng/mL with a peak trough fluctuation of no more than 100%. 189. A system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user; and b) a digital device of an authorized user in communication with the drug delivery device to allow the authorized user to configure, modify, or monitor the dosage regimen; wherein the dosage regimen provides an average ketamine plasma concentration of at least 1 ng/mL with a peak trough fluctuation of no more than 100%. 190. A method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is not configurable by the subject; wherein the dosage regimen comprises periodic doses that provide a clinically effective ketamine plasma concentration with a peak trough fluctuation of no more than 100%. 191. A drug delivery device comprising: a) a reservoir for storing a drug formulation comprising ketamine; b) an infusion pump connected to the reservoir and configured for subcutaneous infusion of the drug formulation; and c) a user interface enabling a subject to self-administer a dose of the drug formulation according to a pre-programmed dosage regimen that is configurable only by an authorized user; wherein the dosage regimen comprises periodic doses that provide a clinically effective ketamine plasma concentration with a peak trough fluctuation of no more than 100%. 192. A system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user; and b) a digital device of an authorized user in communication with the drug delivery device to allow the authorized user to configure, modify, or monitor the dosage regimen; wherein the dosage regimen comprises periodic doses that provide a clinically effective ketamine plasma concentration with a peak trough fluctuation of no more than 100%. 193. A method for self-treatment by a subject outside of a hospital or clinical setting, comprising: a) obtaining a drug delivery device for administering a dose of a drug formulation comprising an NMDA receptor antagonist; and b) self-administering the dose according to a pre-programmed dosage regimen that is not configurable by the subject; wherein the drug delivery device is programmed to restrict administration of a bolus of the drug formulation that exceeds a pre-programmed dosage limit. 194. A drug delivery device comprising: a) a receptacle for receiving a cartridge storing a drug formulation comprising ketamine; b) an infusion pump connected to the receptacle and configured for subcutaneous infusion of the drug formulation; and c) a user interface enabling a subject to self-administer a dose of the drug formulation according to a pre-programmed dosage regimen that is configurable only by an authorized user; wherein the drug delivery device is programmed to restrict administration of a bolus of the drug formulation that exceeds a pre-programmed dosage limit. 195. A system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user; and b) a digital device of an authorized user in communication with the drug delivery device to allow the authorized user to configure, modify, or monitor the dosage regimen; wherein the drug delivery device is programmed to restrict administration of a bolus of the drug formulation that exceeds a pre-programmed dosage limit. 196. A method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is configurable only by an authorized user who is not the subject; wherein the drug delivery device is programmed to allow at-home administration of the drug formulation, wherein the drug delivery device is configured to be tamper-resistant to deter the subject from deviating from the pre-programmed dosage regimen. 197. A drug delivery device comprising: a) a receptacle for receiving a cartridge storing a drug formulation comprising ketamine; b) an infusion pump connected to the receptacle and configured for subcutaneous infusion of the drug formulation; and c) a user interface enabling a subject to self-administer a dose of the drug formulation according to a pre-programmed dosage regimen that is configurable only by an authorized user who is not the subject; wherein the drug delivery device is programmed to allow at-home administration of the drug formulation, wherein the drug delivery device is configured to be tamper-resistant to deter the subject from deviating from the pre-programmed dosage regimen. 198. A system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user who is not the subject; and b) a digital device of an authorized user in communication with the drug delivery device to allow the authorized user to configure, modify, or monitor the dosage regimen; wherein the drug delivery device is programmed to allow at-home administration of the drug formulation, wherein the drug delivery device is configured to be tamper-resistant to deter the subject from deviating from the pre-programmed dosage regimen. 199. A method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is configurable only by an authorized user who is not the subject; wherein the drug delivery device restricts access to the drug formulation to deter usage that deviates from the pre-programmed dosage regimen. 200. A drug delivery device comprising: a) a reservoir for storing a drug formulation comprising ketamine; b) an infusion pump connected to the reservoir and configured for subcutaneous infusion of the drug formulation; and c) a user interface enabling a subject to self-administer a dose of the drug formulation according to a pre-programmed dosage regimen that is configurable only by an authorized user who is not the subject; wherein the drug delivery device restricts access to the drug formulation to deter usage that deviates from the pre-programmed dosage regimen. 201. A system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user who is not the subject; and b) a digital device of an authorized user in communication with the drug delivery device to allow the authorized user to configure, modify, or monitor the dosage regimen; wherein the drug delivery device restricts access to the drug formulation to deter usage that deviates from the pre-programmed dosage regimen. 202. A method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is configurable only by an authorized user who is not the subject; wherein the dosage regimen provides a plasma concentration of ketamine that continuously remains is no lower than a minimum effective concentration and below a minimum toxic concentration for at least 1 week. 203. A drug delivery device comprising: a) a storage chamber storing drug formulation comprising ketamine; b) an infusion pump connected to the reservoir and configured for subcutaneous infusion of the drug formulation; and c) a user interface enabling a subject to self-administer a dose of the drug formulation according to a pre-programmed dosage regimen that is configurable only by an authorized user who is not the subject; wherein the dosage regimen provides a plasma concentration of ketamine that continuously remains is no lower than a minimum effective concentration and below a minimum toxic concentration for at least 1 week. 204. A system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user who is not the subject; and b) a digital device of an authorized user in communication with the drug delivery device to allow the authorized user to configure, modify, or monitor the dosage regimen; wherein the dosage regimen provides a plasma concentration of ketamine that continuously remains is no lower than a minimum effective concentration and below a minimum toxic concentration for at least 1 week. 205. A method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is configurable only by an authorized user who is not the subject; wherein the dosage regimen comprises an initial loading dose and a series of maintenance doses to maintain an effective plasma concentration of ketamine. 206. A drug delivery device comprising: a) a storage chamber storing drug formulation comprising ketamine; b) an infusion pump connected to the reservoir and configured for subcutaneous infusion of the drug formulation; and c) a user interface enabling a subject to self-administer a dose of the drug formulation and according to a pre-programmed dosage regimen that is configurable only by an authorized user who is not the subject; wherein the dosage regimen comprises an initial loading dose and a series of maintenance doses to maintain an effective plasma concentration of ketamine. 207. A system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user who is not the subject; and b) a digital device of an authorized user in communication with the drug delivery device to allow the authorized user to configure, modify, or monitor the dosage regimen; wherein the dosage regimen comprises an initial loading dose and a series of maintenance doses to maintain an effective plasma concentration of ketamine. 208. A method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is configurable only by an authorized user who is not the subject; wherein the dosage regimen comprises at least 3 doses a week to maintain an effective plasma concentration of ketamine through at-home administration of the drug formulation. 209. A drug delivery device comprising: a) a storage chamber storing a drug formulation comprising ketamine; b) an infusion pump connected to the reservoir and configured for subcutaneous infusion of the drug formulation; and c) a user interface enabling a subject to self-administer a dose of the drug formulation and according to a pre-programmed dosage regimen that is configurable only by an authorized user who is not the subject; wherein the dosage regimen comprises at least 3 doses a week to maintain an effective plasma concentration of ketamine through at-home administration of the drug formulation. 210. A system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user who is not the subject; and b) a digital device of an authorized user in communication with the drug delivery device to allow the authorized user to configure, modify, or monitor the dosage regimen; wherein the dosage regimen comprises at least 3 doses a week to maintain an effective plasma concentration of ketamine through at-home administration of the drug formulation. 211. A method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is configurable only by an authorized user who is not the subject; wherein the dosage regimen allows the subject to reach a steady state plasma concentration of ketamine with a peak trough fluctuation percentage of no more than 30% within one day of initiating the dosage regimen. 212. A drug delivery device comprising: a) a storage chamber storing a drug formulation comprising ketamine; b) an infusion pump connected to the reservoir and configured for subcutaneous infusion of the drug formulation; and c) a user interface enabling a subject to self-administer a dose of the drug formulation and according to a pre-programmed dosage regimen that is configurable only by an authorized user who is not the subject; wherein the dosage regimen allows the subject to reach a steady state plasma concentration of ketamine with a peak trough fluctuation percentage of no more than 30% within one day of initiating the dosage regimen. 213. A system comprising: a) a drug delivery device comprising a pump mechanism for administering a drug formulation comprising ketamine and a user interface allowing a subject to self-administer a dose of the drug formulation according to a pre-programmed dosage regimen that configurable only by an authorized user who is not the subject; and b) a digital device of an authorized user in communication with the drug delivery device to allow the authorized user to configure, modify, or monitor the dosage regimen; wherein the dosage regimen allows the subject to reach a steady state plasma concentration of ketamine with a peak trough fluctuation percentage of no more than 30% within one day of initiating the dosage regimen. 214. A method for self-treatment by a subject outside of a hospital or clinical setting, comprising: a) obtaining a drug delivery device for administering a dose of a drug formulation comprising ketamine; and b) self-administering the dose according to a pre-programmed dosage regimen that is not configurable by the subject; wherein the dosage regimen allows the subject to reach a steady state plasma concentration of ketamine with a peak concentration no greater than 100% of a trough concentration for at least one week. 215. A drug delivery device comprising: a) a pump mechanism configured for subcutaneous delivery of a drug formulation comprising ketamine; and b) a user interface enabling a subject to self-administer a dose of the drug formulation and according to a pre-programmed dosage regimen that is not configurable by the subject; wherein the dosage regimen allows the subject to reach a steady state plasma concentration of ketamine with a peak concentration no greater than 100% of a trough concentration for at least one week. 216. A system comprising: a) a drug delivery device comprising a pump mechanism for administering a drug formulation comprising ketamine and a user interface allowing a subject to self-administer a dose of the drug formulation according to a pre-programmed dosage regimen that is not configurable by the subject; and b) a digital device of an authorized user in communication with the drug delivery device to allow the authorized user to configure, modify, or monitor the dosage regimen; wherein the dosage regimen allows the subject to reach a plasma concentration of ketamine with a peak concentration no greater than 100% of a trough concentration for at least one week. 217. A pharmaceutical composition, comprising: (i) an NMDA receptor antagonist, or a hydrate, solvate, or pharmaceutically acceptable salt thereof; and (ii) at least one pharmaceutically acceptable excipient, wherein the pharmaceutical composition is in a form for dosing or administration by intravenous (I.V.), intramuscular, subcutaneous, or intradermal injection. 218. The pharmaceutical composition of embodiment 217, wherein the at least one pharmaceutically acceptable excipient is (i) a surface-active agent, (ii) a non-ionic surfactant, (iii) a phospholipid solubilization agent, (iv) a cyclodextrin excipient, (v) an emulsion stabilizer, (vi) a preservative, (vii) an antimicrobial agent, or (viii) a topical analgesic. 219. The pharmaceutical composition of embodiment 217 or 218, wherein the form is an I.V. dosage form. 220. The pharmaceutical composition of any one of embodiments 217-219, wherein the NMDA receptor antagonist is an arylcyclohexylamine or arylcyclohexylamine derivative. 221. The pharmaceutical composition of any one of embodiments 217-219, wherein the NMDA receptor antagonist also acts as a dopamine reuptake inhibitor, μ-opioid receptor agonist, σ receptor agonist, nACh receptor antagonist, D2 receptor agonistic, or any combination thereof 222. The pharmaceutical composition of any one of embodiments 217-219, wherein the NMDA receptor antagonist is ketamine, phencyclidine (PCP), 3-MeO-Phencylidine, 4-MeO-Phencyclidine, eticyclidine (PCE), 3-MeO-PCE, methoxetamine (MXE), tiletamine, or tenocyclidine (TCP), or a hydrate, solvate, or pharmaceutically acceptable salt thereof 223. The pharmaceutical composition of any one of embodiments 217-222, wherein the pharmaceutical composition comprises from about 10 mg/mL to about 300 mg/mL of the NMDA receptor antagonist, or a hydrate, solvate, or pharmaceutically acceptable salt thereof. 224. The pharmaceutical composition of any one of embodiments 217-223, wherein the pharmaceutical composition comprises from about 10 mg/mL to about 50 mg/mL of the NMDA receptor antagonist, or a hydrate, solvate, or pharmaceutically acceptable salt thereof. 225. The pharmaceutical composition of any one of embodiments 217-224, wherein the pharmaceutical composition comprises about 10 mg/mL, about 15 mg/mL, about 20 mg/mL, about 25 mg/mL, about 30 mg/mL, about 35 mg/mL, about 40 mg/mL, about 45 mg/mL, or about 50 mg/mL of ketamine, or a hydrate, solvate, or pharmaceutically acceptable salt thereof. 226. The pharmaceutical composition of any one of embodiments 217-222, wherein the pharmaceutical composition comprises up to about 300 mg/mL of ketamine, or a hydrate, solvate, or pharmaceutically acceptable salt thereof 227. The pharmaceutical composition of any one of embodiments 217-226, wherein the pharmaceutical composition comprises a pH of about 3.5 to 7.5. 228. The pharmaceutical composition of any one of embodiments 217-227, wherein the pharmaceutical composition comprises a pH of about 5.5 to 7.0. 229. The pharmaceutical composition of any one of embodiments 217-228, wherein the pharmaceutical composition comprises a pH of about 5.5, about 5.6, about 5.7, about 5.8, about 5.9, about 6.0, about 6.5, about 6.6, about 6.7, about 6.8, about 6.9, or about 7.0. 230. The pharmaceutical composition of any one of embodiments 217-229, wherein the dosage form comprises a co-solvent. 231. The pharmaceutical composition of embodiment 230, wherein the co-solvent comprises PEG200, PEG300, PEG400, PEG600, propylene glycol, ethanol, polysorbate 20, polysorbate 80, cremephor, glycerin, benzyl alcohol, dimethylacetamide (DMA), N-methyl-2-pyrrolidone (NMP), tert-butanol, or combinations thereof. 232. The pharmaceutical composition of any one of embodiments 217-231, wherein the dosage form comprises a surface-active agent. 233. The pharmaceutical composition of embodiment 232, wherein the surface-active agent comprises polyoxyethylene sorbitan monooleate (Tween 80), sorbitan monooleate, polyoxyethylene sorbitan monolaurate (Tween 20), lechitin, polyoxyethylene-polyoxypropylene copolymers (Pluronics1), or combinations thereof. 234. The pharmaceutical composition of any one of embodiments 217-233, wherein the dosage form comprises a non-ionic surfactant. 235. The pharmaceutical composition of embodiment 234, wherein the non-ionic surfactant comprises Cremophor RH40, Cremophor RH60, d-alpha-topopherol polyethylene glycol 1000 succinate, polysorbate 20, polysorbate 80, Solutol HS 15, sorbitan monooleate, poloxamer 407, Labrafil M-1944CS, Labrafil M-2125CS, Labrasol, Gellucire 44/14, Softigen 767, or combinations thereof 236. The pharmaceutical composition of any one of embodiments 217-235, further comprising at least one additional active agent that mitigates the side effects of the NMDA receptor antagonist. 237. The pharmaceutical composition of embodiment 236, wherein the at least one additional active agent is a benzodiazepine, a selective serotonin 5-HT3 receptor antagonist, a beta blocker, an inhibitor of CYP2B6 and/or CYP3A and/or CYP2C9, or combinations thereof. 238. The pharmaceutical composition of embodiment 236 or 237, wherein the at least one additional active agent is a benzodiazepine, a selective serotonin 5-HT3 receptor antagonist, or a beta blocker. 239. The pharmaceutical composition of embodiment 236 or 237, wherein the benzodiazepine is lorazepam or midazolam. 240. The pharmaceutical composition of embodiment 236 or 237, wherein the beta blocker is propranolol or atenolol. 241. The pharmaceutical composition of embodiment 236 or 237, wherein the selective 5-HT3 receptor antagonist is ondansetron. 242. The pharmaceutical composition of embodiment 237, wherein the at least one additional active agent is an inhibitor of CYP2B6 and/or CYP3A and/or CYP2C9. 243. The pharmaceutical composition of embodiment 237 or 242, wherein the inhibitor of CYP2B6 is clopidogrel, ticlopidine, orphenadrine, candesartan, amlodipine, felodipine, memantine, clotrimazole, voriconazole, azelastine, clopidogrel, clofibrate, fenofibrate, 2-phenyl-2-(1-piperidinyl)propane, resveratrol, alpha-viniferin, epsilon-viniferin or pregabalin. 244. The pharmaceutical composition of any one of embodiments 237 or 242-243, wherein the inhibitor of CYP3A is nefazodone, aprepitant, fluvoxamine, itraconazole, verapamil, orphenadrine, bergamottin, mibefradil, ketoconazole, itraconazole, resveratrol, alpha-viniferin, epsilon-viniferin or diltiazem. 245. The pharmaceutical composition of any one of embodiments 217-246, wherein the NMDA receptor antagonist is racemic ketamine, (R)-ketamine, or (S)-ketamine. 246. The pharmaceutical composition of any one of embodiments 217-246, wherein the pharmaceutical composition is administered by the drug delivery device of any one of embodiments 1-60. 247. The pharmaceutical composition of any one of embodiments 217-246, wherein the pharmaceutical composition is administered by the system of any one of embodiments 61-120. 248. A method for self-treatment by a subject outside of a hospital or clinical setting, comprising: (i) obtaining a drug delivery device for administering a dose of the pharmaceutical composition of any one of embodiments 217-246; and (ii) self-administering the dose from a selection of at least one pre-programmed dosage regimen that is not configurable by the subject; wherein the at least one dosage regimen provides an effective steady state drug plasma concentration while reducing side effects. The method of embodiment 248, wherein the at least one dosage regimen provides an effective steady state drug plasma concentration. 249. The method of embodiment 248, wherein the at least one dosage regimen is locked after configuration by an authorized user to deter modification by the subject. 250. The method of embodiment 248, wherein the at least one dosage regimen is locked after configuration by the manufacturer to deter modification by the patient. 251. The method of embodiment 248, wherein the drug delivery device is configured to be tamper-resistant to deter administration of a dose of the pharmaceutical composition that deviates from the at least one dosage regimen. 252. The method of embodiment 248, wherein the pharmaceutical composition is stored in tamper-resistant cartridge. 253. The method of embodiment 248, wherein the drug delivery device comprises a reservoir for storing the pharmaceutical composition prior to administration. 254. The method of embodiment 248, wherein the at least one dosage regimen reduces side effects of the pharmaceutical composition while providing the state drug plasma concentration. 255. The method of embodiment 255, wherein the side effects comprise drug dependence or addiction. 256. The method of embodiment 255, wherein the side effects comprise hallucination, disorientation, dissociation, dizziness, drowsiness, increased heart rate, elevated blood pressure, nausea, vomiting, fatigue, brain fog, confusion, anxiety, distress, shortness of breath or any combination thereof 257. The method of embodiment 248, wherein the drug delivery device deters abuse of the pharmaceutical composition by limiting control of the at least one dosage regimen by the subject. 258. The method of embodiment 248, wherein the drug delivery device is configured to administer the pharmaceutical composition according to the at least one dosage regimen for treating Treatment Resistant Depression. 259. The method of embodiment 248, wherein the drug delivery device is configured to administer the pharmaceutical composition according to the at least one dosage regimen for treating major depressive disorder, treatment resistant major depressive disorder, suicidality, suicidal ideation, dysthymia or persistent depressive disorder, bipolar depressive disorder type I, bipolar depressive disorder type II, chronic pain, eating disorder NOS, pain disorder NOS, panic disorder, post-traumatic stress disorder, obsessive-compulsive disorder, complex regional pain syndrome, reflex sympathetic dystrophy, or any combination thereof 260. The method of embodiment 248, wherein the at least one dosage regimen is configured by an authorized user who is a healthcare provider for the subject. 261. The method of embodiment 248, wherein the at least one dosage regimen is prescribed for the subject by a healthcare provider. 262. The method of embodiment 248, wherein the subject is not authorized to configure or modify the at least one dosage regimen. 263. The method of embodiment 248, wherein the drug delivery device allows limited modification of the at least one dosage regimen by the subject. 264. The method of embodiment 248, wherein the at least one dosage regimen comprises a plurality of dosing options selectable by the subject. 265. The method of embodiment 265, wherein the plurality of dosing options is selected from the group consisting of bolus injection, continuous infusion. 266. The method of embodiment 265, wherein the plurality of dosing options comprises differences in dosage size, dosage rate, infusion duration, or any combination thereof. 267. The method of embodiment 248, further comprising allowing an authorized user to remotely configure or modify the at least one dosage regimen over a network. 268. The method of embodiment 248, further comprising storing information for doses administered by the subject. 269. The method of embodiment 248, further comprising allowing an authorized user to remotely monitor the at least one dosage regimen over a network. 270. The method of embodiment 248, further comprising sending, by the drug delivery device, a request to an authorized user regarding the at least one dosage regimen over a network. 271. The method of embodiment 248, further comprising sending and receiving, by the drug delivery device, information over a network. 272. The method of embodiment 248, further comprising pairing the drug delivery device with a communications device that provides a network connection for communicating with an authorized user. 273. The method of embodiment 248, wherein the at least one dosage regimen comprises a dosage limit setting an upper limit on a size of the dose. 274. The method of embodiment 248, wherein the drug delivery device prohibits administration of a dose of the pharmaceutical composition that exceeds a dosage limit. 275. The method of embodiment 248, wherein the drug delivery device prohibits administration of a dose of the pharmaceutical composition that causes a total daily dose to exceed a daily dosage limit. 276. The method of embodiment 248, wherein the drug delivery device prohibits administration of a dose of the pharmaceutical composition at an infusion rate that exceeds a dosage limit. 277. The method of embodiment 248, wherein the drug delivery device deters abuse of the pharmaceutical composition. 278. The method of embodiment 248, wherein the pump mechanism is configured to administer the pharmaceutical composition through subcutaneous or intramuscular injection. 279. The method of embodiment 248, wherein the dose comprises an infusion rate of at least about 1 mg/hour. 280. The method of embodiment 248, wherein the dose comprises an infusion rate of no more than about 200 mg/hour. 281. The method of embodiment 248, wherein the dose comprises an infusion rate from about 1 mg/hour to about 200 mg/hour. 282. The method of embodiment 248, wherein the dose comprises an infusion of at least about ten (10) minutes. 283. The method of embodiment 248, wherein the dose comprises an infusion that is continuous. 284. The method of embodiment 248, wherein the dose comprises an infusion rate of at least lmg/hour for at least ten (10) minutes. 285. The method of embodiment 248, wherein the NMDA receptor antagonist is a racemic mixture of ketamine. 286. The method of embodiment 248, wherein the NMDA receptor antagonist is substantially pure S-ketamine. 287. The method of embodiment 248, wherein the NMDA receptor antagonist is substantially pure R-ketamine. 288. The method of embodiment 248, wherein the dosage regimen provides a clinically effective steady-state concentration of the NMDA receptor antagonist outside of a hospital or clinical setting. 289. The method of embodiment 248, wherein the dosage regimen provides a clinically effective steady-state concentration of the NMDA receptor antagonist for at least 1 week. 290. The method of embodiment 248, wherein the dosage regimen provides an average treatment steady state plasma concentration of at least 1 ng/mL with a peak trough fluctuation of no more than 100% of the average steady state plasma concentration during treatment. 291. The method of embodiment 248, wherein the at least one dosage regimen provides a clinically effective steady-state concentration of the NMDA receptor antagonist with peak trough fluctuation of no more than 100% while the steady-state plasma concentration is maintained. 292. The method of embodiment 248, wherein the at least one dosage regimen provides a clinically effective steady-state concentration of the NMDA receptor antagonist with a Cmax to Cmin ratio of no more than 4. 293. The method of embodiment 248, wherein the at least one dosage regimen provides a concentration of the NMDA receptor antagonist of at least 1 ng/mL throughout a duration of the at least one dosage regimen. 294. The method of embodiment 248, wherein the at least one dosage regimen comprises at least 1 dose per month. 295. The method of embodiment 248, wherein the at least one dosage regimen comprises a single continuous dose. 296. The method of embodiment 248, wherein the at least one dosage regimen comprises a loading dose and a series of maintenance doses. 297. The method of embodiment 248, wherein the at least one dosage regimen comprises periodic doses. 298. The method of embodiment 248, wherein the at least one dosage regimen comprises aperiodic doses. 299. The method of any of embodiments 248-299, wherein the method is used for treating, preventing, or ameliorating at least one symptom of a disorder, disease, or condition. 300. The method of embodiment 300, wherein the disorder, disease, or condition is a mental or psychiatric disorder, a neurological condition or disorder, pain, or an inflammatory disorder. 301. The method of embodiment 300, wherein the disorder, disease, or condition is pain. 302. The method of embodiment 300, wherein the neurological condition or disorder is chronic pain. 303. The method of embodiment 300, wherein the disorder, disease, or condition is a mental or psychiatric disorder. 304. The method of embodiment 300, wherein the mental or psychiatric disorder is Major Depressive Disorder, treatment resistant major depressive disorder, suicidality, suicidal ideation, Substance-Related Disorder, Sedative-, Hypnotic-, or Anxiolytic-Related Disorder, Sedative-, hypnotic-, or anxiolytic withdrawal, alcohol withdrawal, cannabis dependence, cannabis withdrawal, barbiturate dependence, barbiturate withdrawal, benzodiazepine dependence, benzodiazepine withdrawal, amphetamine dependence, amphetamine withdrawal, opioid dependence, opioid withdrawal, opioid-related disorder, alcohol dependence, cocaine dependence, or cocaine withdrawal. 305. A sealed reusable delivery and control system comprising the drug delivery device of any of embodiments 1-216. 306. A sealed reusable delivery and control system comprising drug delivery device of any of embodiments 1-216, comprising: a) a control system providing a multi-tiered security authentication configured to use one or more of a patient ID, physician ID, Delivery device ID, Mobile device ID, cartridge ID that is compared a database via security challenges; b) an energy harvesting NFC tag for cartridge ID; c) one or more accumulation registers; and d) one or more tamper proof conductors. 307. The system of embodiment 306, wherein the delivery device determines its location on a body by reading body resistance. 308. The system of embodiment 306, wherein the delivery device comprises one or more capacitive sensors to identify body presence and identify pain by user-entered pattern for logging and dose delivery. 309. The system of embodiment 306, wherein the delivery device electrically isolates the cartridge from the sealed while enabling communications, mechanical interfaces magnetically and electromagnetically. 310. The system of embodiment 306, wherein the control system protects and secures the dosage by tracking dose on a cartridge and the drug delivery device. 311. The system of embodiment 306, wherein the system is configured to protect and secure a pharmaceutical formulation through physical features that create tamper resistance around a drug reservoir stored within the drug delivery device. 312. The system of embodiment 306, wherein the drug delivery device is sealed and charged wirelessly. 313. The system of embodiment 306, wherein the system is configured for delivery of pain analytics to a physician of a patient during pain relief delivery. 314. The system of embodiment 313, wherein the pain analytics comprises heart rate, resistance, breath rate, pain rating, or any combination thereof to a physician of the patient. 315. A method to deliver pain relief using the drug delivery device of any of embodiments 1-216 or 306-314.

### EXAMPLES

### Example 1—at-Home Drug Delivery

Depression

A patient suffering from treatment-resistant depression is prescribed ketamine HCl. The patient is first treated with ketamine by intravenous infusion at his psychiatrist's clinic. The psychiatrist works with the subject to determine the effective dosing and infusion rate and the optimal dosing frequency of about 50 mg during a 40 minute infusion for achieving clinical remission of depression during the course of this treatment. However, because the visits effectively limit the patient to a single dose (50 mg) over the course of a 40 minute infusion on each treatment day, he experiences some side effects of the large dose including nausea and disorientation. Moreover, the short half-life of ketamine means that the beneficial effect of a morning dose wears off by the late afternoon to early evening, or that longer duration of infusion is difficult due to office based practice realities and the demands of the patient's life. Because of the side effects, the short beneficial duration, the minimal options regarding dosage and infusion rates, and the inconvenience of daily visits, the patient requests an alternative mode of treatment. Accordingly, the psychiatrist prescribes an at-home dosage regimen using a programmable drug delivery device. The psychiatrist enters an authentication code through the interface of the drug delivery device to unlock the device and then configures the dosage regimen to administer one or more infusions of ketamine at a rate (e.g. mg/hour), total treatment dose (e.g., 50 mg/treatment), and frequency (e.g., 3 treatments/week) that is reasonably expected to produce an effective response or has been has previously been effective for the patient, and that may include a baseline infusion rate of 0.1 to 2 mg/hour to keep the catheter patent during an extended period of treatment. Concerned with the rising epidemic of recreational ketamine use, the psychiatrist sets strict dosage limits that prohibit any deviation above the frequency, and/or the continuous infusion rate, and/or the treatment infusion rate, and/or the total dose per treatment. The psychiatrist then locks the device to prevent any further modification of the dosage regimen. The psychiatrist also writes a re-fillable prescription that lets the patient obtain disposable cartridges containing a ketamine HCl formulation. Once at home, the patient inserts a cannula connected to the drug delivery device into the side of his abdomen. Next, the patient inserts a disposable cartridge into the drug delivery device and uses the user interface to enter instructions to initiate the treatments according to the programmed dosage regimen. Due to the strict limits set by the psychiatrist, the patient is unable to adjust the continuous infusion rate above the preset 0.1-2 mg/hour rate, the frequency with which treatment infusions occur or the treatment infusion rate, and total dose given with each treatment, although he is given the option of reducing any of these parameters. The pump mechanism of the device then begins pumping the ketamine formulation as programmed. The infusion of ketamine HCl maintains an effective plasma concentration of ketamine sufficient to alleviate the patient's symptoms while staying below concentrations that evoke significant nausea, dissociation and disorientation.

Pain Management

Another patient suffering from pain associated with Ehlers Danlos Syndrome Type III is provided with the same programmable drug delivery device described above for treating depression. Because the patient tells his doctor that he tends to experience pain spikes during the evening, his doctor programs the dosage regimen to allow the patient to increase the dosage of the bolus or continuous infusion rate to a higher level during the evening than during the day. In addition, the patient optionally accesses the interface of the drug delivery device to provide a self-rated pain scale. The patient enters his personal pain rating on a 1-10 scale as around a 2-3 during the day. During administration of the ketamine, the patient experiences reduced pain of 1-2 and enters this information into the drug delivery device. At night, the patient enters higher pain ratings from 4-6. The ketamine dosage is able to lower the pain to 2-4. This information is transmitted routed to the patient's mobile phone which has a mobile app configured to communicate with the drug delivery device. The mobile app uploads the self-assessed pain ratings to an online database accessible by the doctor. The patient also uses the mobile app to send a message to the doctor requesting the infusion rate threshold be raised. Upon seeing that the patient is still experiencing pain levels of 2-4, the doctor updates the dosage regimen remotely to enable the patient to raise the infusion rate above a preset threshold. The patient then increases the infusion rate. His pain decreases, and he enters self-assessed pain ratings of 1-2.

### Example 2—Tamper Resistant Drug Cartridge and/or Programming

A patient suffering from treatment-resistant depression is prescribed an at-home dosage regimen of ketamine HCl using a programmable drug delivery device. The psychiatrist presses his finger against a biometric fingerprint reader on the interface of the drug delivery device, or enters a password code to unlock the device and then configures the dosage regimen to administer ketamine at an effective frequency, dose and infusion rate. Concerned with the rising epidemic of recreational ketamine use, the psychiatrist considers setting strict dosage limits that prohibit any deviation above the parameters entered. However, the patient convinces the psychiatrist that he may need a higher dosage rate during certain scenarios when his symptoms are more severe. Therefore, the psychiatrist sets a more lenient dosage limit that allows the patient to increase the infusion rate or total dose but also sets a total daily ketamine dosage limit of 50 mg. The psychiatrist then locks the device to prevent any further modification of the dosage regimen. In addition, the psychiatrist configures the device to send periodic updates for device usage information to a remote server accessible by the psychiatrist in order to remotely monitor usage of the device. The psychiatrist also writes a re-fillable prescription that lets the patient obtain disposable cartridges containing a ketamine HCl formulation, refills of the device directly at the pharmacy or new pump units with the ketamine HCl formulation already sealed within the unit. Once at home, the patient inserts a cannula attached to a tube in connection with the drug delivery device into the side of his abdomen. Next, the patient inserts a disposable cartridge into the drug delivery device and uses the biometric fingerprint reader or password to initiate the treatment according to the programmed dosage regimen. The pump mechanism of the device then begins pumping the ketamine formulation at the programmed infusion rate for the programmed treatment period. The patient feels a euphoric effect from the ketamine and tries to enhance this sensation by increasing the dosage. He is unable to increase the infusion rate or total mg infused in each treatment beyond the dosage limit set by the psychiatrist. After a preprogrammed period of time, the daily dosage limit of 50 mg/24 hours is reached, and the device stops administering ketamine. The patient attempts to modify the dosage regimen to increase the dosage rate and the dosage limits, but is unable to make any changes because the device is locked and his fingerprint or password does not have authorization to unlock the device. The patient then attempts to break open a disposable cartridge with a knife. However, the cartridge is made of heavy plastic that resists tampering and the device records that the cartridge was removed before it was fully used, leading to the device being locked and unusable by the patient until it is unlocked by the psychiatrist. The psychiatrist is also able to monitor the repeated attempts to increase the dosage rate and dosage limits by logging onto the remote server via a web API. The psychiatrist is able to see the time, duration, dosage rate, total dose administered, remaining dose, and other data regarding device usage. Depending on the circumstance and the clinical judgement of the psychiatrist, the treatment can then be stopped or continued and/or the patient can be referred to other treatments.

Another patient suffering from chronic pain is given a programmable drug delivery device comprising a pre-programmed dosage regimen as described above.

### Example 3—Multiple Dosage Regimens

A patient suffering from treatment-resistant depression is prescribed at-home administration of ketamine HCl using a programmable drug delivery device. The doctor is concerned about prescribing doses that are too low or too high for the patient because he has not previously prescribed ketamine for him before. The doctor decides to program the drug delivery device with three dosage regimens that the patient can select from. The dosage regimens provide continuous infusion subcutaneous or intramuscular delivery of ketamine HCl at 8 mg/hour for two hours, 15 mg/hour for two hours, or 25 mg/hour for two hours. Back at home, the patient accesses the drug delivery device by pressing his thumb against the biometric fingerprint reader of the drug delivery device or entering a password. The patient selects the dosing button on the device interface, which presents him with the three dosage regimens. The patient first selects the lowest dosage regimen, and initiates a treatment. After an hour, the patient feels that the dosage regimen is not working appropriately, and switches to the next highest dosage regimen. This higher dosage is effective, and the patient decides against further switching the dosage regimen.

Another patient suffering from chronic pain is given a programmable drug delivery device comprising a pre-programmed dosage regimen as described above.

### Example 4—Multiple Frequency Regimens

A patient suffering from treatment-resistant depression is prescribed at-home administration of ketamine HCl using a programmable drug delivery device. The doctor is concerned about prescribing treatments at a frequency that is too low or too high for the patient because he has not previously prescribed ketamine for him before. The doctor decides to program the drug delivery device with the option to initiate a treatment from one time to no more than 4 times per 7 days with no more than one treatment per 16 hours. Back at home, the patient accesses the drug delivery device by pressing his thumb against the biometric fingerprint reader of the drug delivery device or entering a password. The patient selects the dosing button on the device interface, which presents him with the preprogrammed dosage regimens and initiates treatment. The patient experiences some euphoria with the treatment and decides to initiate a second treatment after only 8 hours but is unable to do this because the device is locked by the dosing frequency rate set by the psychiatrist. The patient must wait until the next day to initiate another treatment and/or make an appointment with the physician to discuss any changes in the treatment regimen.

Another patient suffering from chronic pain is also given a programmable drug delivery device comprising a pre-programmed dosage regimen as described above.

### Example 5—Emulsifiers

There may be a higher percentage of ketamine in unionized form relative to lower pH values, once the pH of solution is adjusted upward to an average of from 5 to 7.5 pH. As the unionized form (freebase) can come out of solution it may be necessary to include an excipient(s) associated with solubility enhancement, such co-solvents, solubilization agents (including surface active agents and complexation agents) and stabilization agents (including buffers). Additional additives to the formulation include antioxidants, cryoprotectants, lyoprotectants, bulking agents, tonicity-adjusting agents and antimicrobial preservative agents. Co-solvents contemplated for use in some formulations described herein include, but are not limited to propylene glycol, glycerin, ethanol, polyethylene glycol (300, 400, 600, 3350, and 4000), sorbitol, dimethyl acetamide, Cremophor EL, N-methyl-2-pyrrolidone, dimethylsulfoxide, glycofurol, benzyl alcohol, ethyl lactate, cetyl alcohol, and/or N-methylpyrrolidone. In some formulations, the potential solubilization agents might include surface-active agents. Surface-active agents that were considered include: Polyoxyethylene sorbitan monooleate (Tween 80), Sorbitan monooleate (polysorbate 80), Polyoxyethylene sorbitan monolaurate (Tween 20), Lecithin, Polyoxyethylene-polyoxypropylene copolymers (Pluronics1). In some formulations the potential solubilization agents might include non-ionic surfactants. Non-ionic surfactants that were considered include: Cremophor RH 40, Cremophor RH 60, d-alpha-tocopherol polyethylene glycol 1000 succinate, polysorbate 20, polysorbate 80, Solutol HS 15, sorbitan esters (e.g., sorbitan monooleate, sorbitan monolaurate, sorbitan monostearate, sorbitan monopalmitate, sorbitan trioleate, sorbitan tri stearate), poloxamer 407, Labrafil M-1944C S, Labrafil M-2125CS, Labrasol, Gellucire 44/14, Softigen 767, mono-fatty esters and di-fatty acid esters of PEG 300, 400, and 1750, and/or tyloxapol. In some formulations, potential solubilization agents that were considered include phospholipids such as: hydrogenated soy phosphatidylcholine, phosphatidylcholine, distearoylphosphatidylglycerol, L-alpha-dimyristoylphosphatidylcholine, and L-alpha-dimyristoylphosphatidylglycerol. In some formulations, the potential solubilization agents that were considered include complexation agents such as: Hydroxypropyl-beta-cyclodextrin, Sulfobutylether-beta-cyclodextrin (Captisol1), Polyvinylpyrrolidone, amino acids such as arginine, lysine, and histidine, and other cyclodextrins.

There are cyclodextrin excipients that exhibit little pharmacologic activity on their own. These are used to enhance the stability, tolerability, and absorption of compounds in parenteral application. In some formulations, the potential cyclodextran solubilization agents that were considered include: alpha-Cyclodextrin (alpha-CD), beta-Cyclodextrin (beta-CD), gamma-cyclodextrin, c-Cyclodextrin (c-CD), Diethyl-ethyl-beta-cyclodextrin (DE-beta-CD), Dimethyl-ethyl-beta-cyclodextrin (DM-beta-CD), Hydroxypropyl-beta-cyclodextrin (HP-beta-CD), Hydroxypropyl-gamma-cyclodextrin (HP-gamma-CD), methyl-beta-cyclodextrin (M-beta-CD), Sulfobutylether-beta-cyclodextrin (SBE-beta-CD), randomly methylated beta-cyclodextrin (RM-beta-CD), maltosyl-beta-cyclodextrin (mal-beta-CD), and hydroxylpropyl-alpha-cyclodextrin.

In some formulations, the potential stabilization agents that are contemplated include buffers: Acetate, Citrate, Sodium Citrate, Tartrate, Phosphate, histidine, bicarbonate, Triethanolamine (TRIS) and their salts. In some formulations, the potential stabilization agents might include antioxidants and preservatives such as: Ascorbic acid, Acetylcysteine (NAC), Sulfurous acid salts (bisulfate, metabisulfite), Monothioglyercol. Butylated hydroxyanisole (BHA), Butylated hydroxytoluene (BHT), Tert-butylhydroquinone (TBHQ), 2′,4′,5′-Trihydroxybutyrophenone phenylhydrazone (THBP), Ethylenediaminetetraacetic acid (EDTA), Sodium formaldehyde sulfoxylate (SFS), Tocopherol (Vitamin E), Ascorbyl palmitate, Gallates (e.g., propyl gallate, octyl gallate, lauryl gallate), Cysteine ethyl ether, Tartaric acid, Phosphoric acid, Thiourea, Sodium thioglycolate, Nitrogen, and/or Argon.

In some formulations, the potential stabilization agents might include bulking agents, cryoprotectants, and lyoprotectants. Agents that were considered include: Mannitol, Glycine, Sucrose, Lactose, Trehalose, Dextran, Povidone, Sorbitol and/or Polydextrose. In some formulations potential stabilization agents might include tonicity-adjusting agents. Agents that were considered include: sodium chloride, Glycerin, Mannitol, Dextrose, and/or glycerol. In some formulations the potential stabilization agents might include antimicrobial agents including, but not limited to: Phenol, Meta-cresol, Benzyl alcohol, parabens (methyl, propyl, or butyl), benzalkonium chloride, chlorobutanol, Myristyl gamma picolinium chloride, 2-phenoxyethanol, Phenethyl alcohol, Sorbates (sorbic acid, sodium sorbate), Ethanol, and/or Propylene glycol.

In some formulations, soothing agents might include topical analgesics such as: lidocaine, benzocaine, tetracaine, bupivicaine, ropivacaine, and/or levobupivacaine.

In some formulations, emulsion stabilizers include hydroxyethyl cellulose, hydroxypropylcellulose, and/or hydroxypropyl methyl cellulose (hypromellose).

These formulations are suitable for subcutaneous administration using a programmable drug delivery device.

### Example 6—pH Adjustment to Ketamine

Ketamine is generally produced with a pH that ranges from pH 3.5-5.5. Subcutaneous delivery of ketamine by bolus or basal rate infusion can create local irritation of tissues acutely and over time. As such, it will be of likely benefit in clinical application of ketamine via subcutaneous delivery to adjust pH from an average of 4.5 to an average of 6.5 (range 5-7.5).

Adjusting the pH of lidocaine is beneficial to decrease the pain in local target delivery tissues both during and after injection. Additionally, buffering of local anesthetics will reduce the pain of local anesthetic injection.

As the pH of the solution approaches a neutral pH of 7, it is likely that a substantial fraction of ketamine in solution would be present in its unionized form rather than as the normal ionized form (HCl salt). As the unionized form (freebase) is less soluble in water than the ionized form (HCl salt) a strategy to ensure aqueous solubility is such as emulsification, stabilization, and complexation with industry standard and innovative emulsifiers and stabilizers can be employed.

### Example 7—Detection of Gross Analgesic Activity in the Rat Using a Single Subcutaneous Dose of Ketamine Via a Motorized Insulin Pump

1.1 Objective

The purpose of this study was to detect gross analgesic activity in the rat and as such the highest anticipated dose of Ketamine was used. Comparisons were made to pre-dose values and/or vehicle background data. A battery of tests was performed including tail flick, Randell Selitto and writhing test:

tail flick—central acting

Randall Selitto—hyperalgesic activity

writhing test (abdominal spasm)—peripherally acting

1.2 Pain

Acute and chronic pain remains a global health problem despite remarkable progress in the understanding of its mechanisms. However, regardless of its prevalence, it is a very difficult phenomenon to treat, and only small preclinical advances have been effectively translated into the clinical setting (Gregory et al., 2013). Animal models evaluate two main symptoms of pain: hyperalgesia and allodynia or overt nociception (the nervous systems response to pain).

The ability to detect a harmful stimulus is a fundamental physiological function in mammals. However, it has become evident that nociception is a heterogeneous spectacle that differs extensively based on the affected tissue and mechanism of injury (Ness and Gebhart, 1990; Sluka, 2002; Hoeger et al., 2007; DeSantana and Sluka, 2008; Milligan and Watkins, 2009; Schmidt et al., 2010).

Animals cannot be said to be reporting pain; therefore, any reaction to a stimuli does not evidence the experience of pain (Sankuhler, 2009). Consequently, no single model can directly measure pain in animals, and, as such, pain is inferred from pain-like behaviors, such as withdrawal from the nociceptive stimulus. If a stimulus is applied that does not normally evoke a pain response and the animal withdraws, the animal has allodynia, and if the animal withdraws with an exaggerated response, it has hyperalgesia. However, it is difficult to distinguish between allodynia and hyperalgesia in animals. Both are the result of sensitization of nociceptors in which, for example, inflammatory mediators activate second messenger pathways, with subsequent phosphorylation of the voltage-dependent sodium channels and inhibition of the voltage dependent potassium channels that result in the lowering of the nociceptor threshold and increased neuronal membrane excitability (Verr et al., 2006).

It has, therefore, been necessary to develop indirect reliable, reproducible, sensitive, and specific methods to quantify and evaluate pain-like behaviors in animals (Mogil, 2009). Animal models of nociception have been crucial in understanding the complex mechanisms of pain. The most appropriate models should create nociception by mimicking the mechanisms of specific clinical conditions. Equally, measures of pain-like behavior must not only detect pain-like responses, but should do so in a way that is consistent with the experience of pain in the clinical setting (Gregory et al., 2013).

Animal models of pain have two important components: the method of injury and end-point measurement. These models can be divided into stimulus evoked (mechanical, heat or cold, or irritant) and non-stimulus evoked. For the purposes of this study, evoked models were used, including tail flick, Randall Selitto, and abdominal spasm (writhing test) tests.

The tail flick test involves application of a heat stimulus (infrared light) to the tail, and latency to withdrawal from the stimulus is recorded. The response measured is immediate, uses the Aδ- and C-fiber inputs, and is known to activate the spinal dorsal horn, the cells of which are nociceptive-specific. The response has been reported as proportional to the frequency of stimulus and the fiber class of afferent input (Eaton, 2003). However, a similar response has been observed in spinally transected rats, indicating that the tail withdrawal response is a spinal reflex, rather than a pain behavior involved in higher brain centers (Deuis et al., 2017). Conversely, the contribution of supraspinal processing to the tail flick response is dependent on the heating slope of the stimulus that leads to more delayed responses involving higher central nervous system functions believed to be necessary to process pain (Jensen and Yaksh, 1986). Tail-flick tests have been reliably used for determining the potency of opioid analgesics and, as such, can prove valuable for predicting analgesic effects in humans (Grumbach, 1966).

The Randall Selitto test is a tool to assess the response thresholds to mechanical pressure stimulation following induction of a hyperalgesic state by injection of an inflammatory agent and is considered a measure of mechanical hyperalgesia (Randall and Selitto, 1957). This involves application of an increasing force to the surface of the paw until withdrawal or vocalization occurs. The test can produce results similar to decreases on pressure pain thresholds observed in clinical conditions, such as fibromyalgia, myofascial pain, or osteoarthritis (Arendt-Nielsen et al., 2010; Bennett, 2007; Finan et al., 2013; Hsu et al., 2010). Centrally and peripherally acting effects can be detected using this model, for example, opioids increase the mechanical threshold to the normal and inflamed paw, whereas nonsteroidal anti-inflammatory drugs are only effective in the inflamed paw.

In the abdominal spasm (acetic acid-induced writhing) test, acetic acid is injected into the peritoneal cavity where it directly activates nociceptors and results in an abdominal writhing response. This writhing response is characterized by contraction of abdominal muscles followed by an extension of the hind limbs (Collier et al., 1968). This pain-behavior is considered reflexive and evidence of visceral pain associated with visceral chemoreceptors (Hammond, 1989). However, the test lacks specificity as it has been shown to work well for all major and minor analgesics (Loux et al., 1978). It is widely accepted that this model can be used as a screening method to determine the effects of drugs on inflammatory induced nociception (Vogel et al., 1997; Chiba et al., 2008). Although it can be used as a screening tool, it is unclear as to which clinical conditions the use of a chemical irritant represents (Gregory et al., 2013).

1.3 Study Design

Eighteen male Sprague-Dawley rats were obtained from Charles River, Margate, United Kingdom, and were used for dosing. Animals weighed between 319 and 419 g on the day of dosing. Eight male Han Wister rats were obtained from Charles River, Margate, United Kingdom and used for Group 4. Animals weighed between 381 and 504 g on the day of dosing. The animals were assessed according to Table 1.

TABLE 1
Animal Assessments

| Group | Group | Dose level | | |
|---|---|---|---|---|
| number | description | (mg/kg) | Test | Animal numbers |
| 1 | Ketamine | 50 | Randall Selitto | R0001-R0006 |
| 2 | Ketamine | 50 | Tail Flick | R0101-R0106 |
| 3 | Ketamine | 50 | Abdominal | R0201-R0206 |
| | | | Spasm | |
| 4 | Ketamine | 50 | Randall Selitto | R0301-R0308 |

Ketamine has analgesic and antidepressant properties and is ideally suited to treat pain-induced depression (Garcia et al., 2008; Correll et al., 2004). Ketamine antagonizes N-Methyl-D-aspartic acid receptors in spinal dorsal horn neurons to decrease central sensitization, provides descending monoaminergic inhibition, and blocks Na+ channels and μ-opioid receptors in peripheral fibers (Jørum et al., 2003; Sawynok and Reid, 2002; Koizuka et al., 2005). Ketamine is metabolized within an hour and is useful as a short-acting analgesic (Cohen et al., 1973). Although ketamine may block central sensitization to mediate long-acting analgesia, this remains to be proven in clinical practices (Max et al., 1995). In rats, doses of greater than 25 mg/kg are needed for analgesia (Wang et al., 2011).

Rodent studies have established doses of >25 mg/kg as necessary for anti-nociception. The duration of anti-nociception is also variable, but is most often limited to within 24 hours. However, at higher doses of ketamine (50 mg/kg), no relief of evoked pain has been noted 24 hours after administration. Thus, ketamine may not provide long-lasting analgesia for evoked or spontaneous pain at sub-anesthetic doses, but may in the short term (Wang et al., 2011).

The dose level for this study was, therefore, 50 mg/kg.

Animals were dosed subcutaneously via a motorized insulin pump.

1.4 Test Article Information

| Test Article | Storage | Lot Number | Expiry date |
|---|---|---|---|
| Ketamine (Ketamidor, | 15 to 25° C. | 0817683AA | July 2020 |
| 100 mg/ml) | | | |

Ketamine was used as supplied by the manufacturer.

1.5 Inflammatory Agent Information for the Randall-Selitto Test

| Test Article | Storage | Lot Number | Expiry date |
|---|---|---|---|
| Golden Dawn 100 natural | 15 to 25° C. | 54325 | 31 Dec. 2020 |
| Brewer's yeast | | | |

The inflammatory agent was prepared in water as a 20% suspension on the day of dosing.

1.6 Irritant Information for the Abdominal Spasm (Writhing) Test

| Test Article | Storage | Lot Number | Expiry date |
|---|---|---|---|
| 1% Glacial acetic acid | 15 to 25° C. | GLAA132-35 | 28 Feb. 2019 |

The irritant was prepared in water as a 1% solution on the day of dosing.

1.7 Body Weights

Individual body weights were recorded on the day of dosing.

1.8 Dosing

Each animal was shaved around the scapula region to allow attachment (around the scruff area) of the adhesive pad from the infusion set.

Each animal was dosed using the appropriate volume based on body weight. Animals were restrained for a short duration to allow the completion of dosing.

1.9 Tail Flick Assessment

Animals were restrained (e.g., in a restraining tube) on top of the tail flick unit in such a way that the position of the tail tip of the animal was consistently placed.

Appropriate materials (e.g., Blu Tack) were used to guide the tail tip into position. The tail tip was left in position free from the operator and judged as settled before the infrared beam was activated. This minimized the possibility of recording a natural or unrelated tail flick. After activating the infrared beam, the machine automatically registered the time duration to the first flick of the tail.

To prevent unnecessary tissue injury to animals, a cut off time point of 15 seconds was applied (i.e., the tail tip was removed from the infrared window once 15 seconds had passed without a tail flick).

Each animal was tested for its pre-study reaction time to the infrared beam, and the time recorded was maintained in the study data.

Following dosing of each animal, reaction times were measured, as previously described, immediately prior to dosing and at 15 and 30 minutes and 1, 2, and 4 hours post-dose.

Signs of anesthesia were recorded.

Comparisons were made with pre-dose reaction times.

1.10 Randall-Selitto Assessment

The threshold of a pain response to increasing pressure was measured using an analgesy meter (Ugo Basile, Italy). The animal was gently held by a competent operator, and the hind paw of the animal was positioned over a convex surface (a cone-shaped pusher). A gradually increasing pressure (g) was applied to the upper surface of the paw. The force was continuously monitored by a pointer moving along a linear scale and was stopped by depression of a foot pedal when the animal struggled. A cut-off value of 300 g was adopted (based on available literature and experience).

Pre-dose Phase: The pressure pain threshold of each animal was tested on two occasions (once in the morning and once in the afternoon). This allowed animals to acclimate to the procedure.

Day of Dosing: On the day of dosing, each animal was administered a 0.1 mL 20% suspension of Brewers Yeast subcutaneously into the sub-plantar of the right hind paw at approximately 180 minutes prior to ketamine administration. Immediately prior to ketamine administration (Group 4), 15 and 30 minutes and 1, 2, and 4 hours post administration of ketamine, each animal was assessed for a response to increasing pressure on both hind paws (Group 1 used right hind paw, Group 4 used both hind paws), using the analgesy meter. Signs of anesthesia were recorded.

1.11 Writhing Test

At 1 hour post administration of ketamine, each animal was administered a 1 mL intraperitoneal injection of 1% acetic acid. Animals were immediately placed into individual observation chambers, and the number of abdominal spasms elicited over the subsequent 25-minute period was recorded.

Comparisons were made to background data.

Results

The objective of this study was to select a pain model for which ketamine demonstrated efficacy, and, as such, the tests were designed to detect gross effects using a limited number of animals, with comparisons with background data and/or pre-dose values.

2.1 Signs of Anesthesia

Animals were noted to have reduced righting reflex, body tone, and ataxia up to 1 hour post-dose. Signs of anesthesia confirmed delivery of ketamine was achieved. The efficacy of ketamine as an anesthetic is well documented.

These signs were most prevalent and moderate in nature between 15 and 30 minutes post-dose. Group 4 animals displayed signs that were most prevalent and moderate in nature between 0.5 and 1 hour post-dose.

2.2 Tail Flick Test

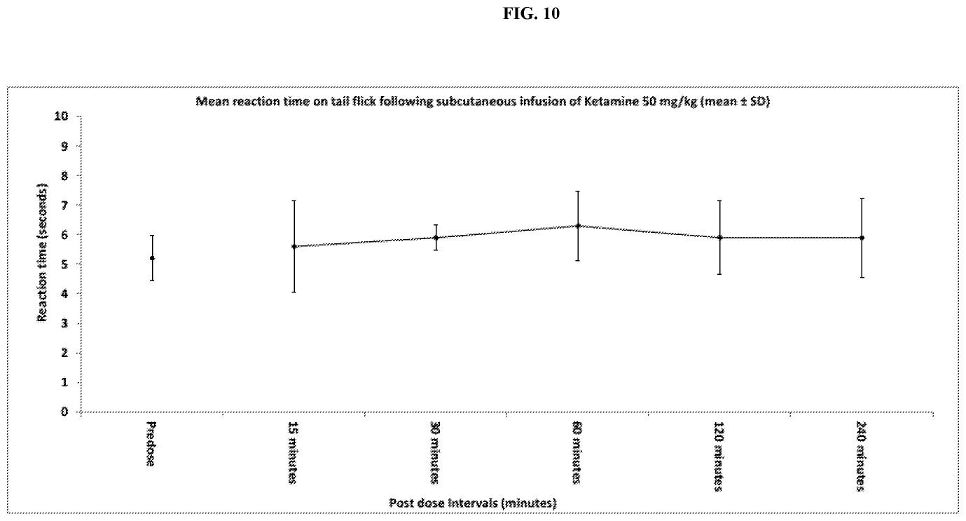

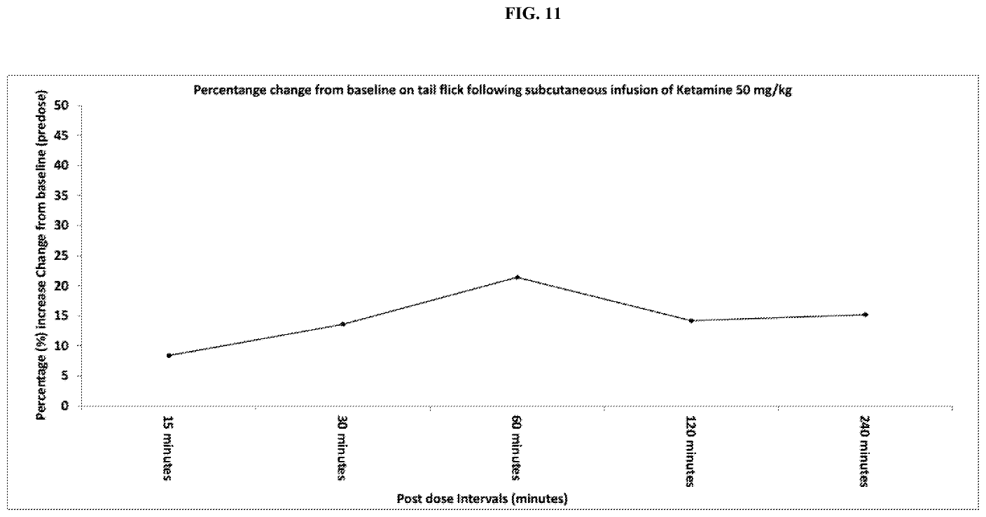

Results are summarized in Table 2 individual animal data are presented in Table 6. The data are graphically represented in FIG. 10 and FIG. 11.

Subcutaneous infusion of 50 mg/kg ketamine produced a slight increase in latency of tail flick at all post-dose time points. The group mean reaction time was longest at 1 hour post-dose (6.3 seconds; a 21.4% increase from baseline of 5.2 seconds). The shortest reaction time was elicited at 15 minutes post-dose (5.6 seconds; an 8.4% increase from baseline). Although the time taken for the sensation of pain (induced by infrared beam) to have been perceived was increased in a time dependent manner, this was of small magnitude (1.1 second) and, as such, was not conclusive of an analgesic response.

TABLE 2
Summary of tail flick assessment

| | Time (s) taken for response at time (h) | | | | | |
|---|---|---|---|---|---|---|
| | post administration of ketamine (50 mg/kg, sc) | | | | | |
| | Pre-dose | 0.25 | 0.5 | 1 | 2 | 4 |
| Mean | 5.2 | 5.6 | 5.9 | 6.3 | 5.9 | 5.9 |
| SD | 0.77 | 1.54 | 0.42 | 1.18 | 1.25 | 1.34 |
| % change from baseline | — | 8.4 | 13.6 | 21.4 | 14.2 | 15.2 |
| (Immediately Prior to | | | | | | |
| dose) | | | | | | |

Mean data for tail flick (n=6, represented as mean±standard deviation SD)

Time (s) taken for response at time (h) post administration of ketamine (50 mg/kg, sc)

2.3 Randall-Selitto Test

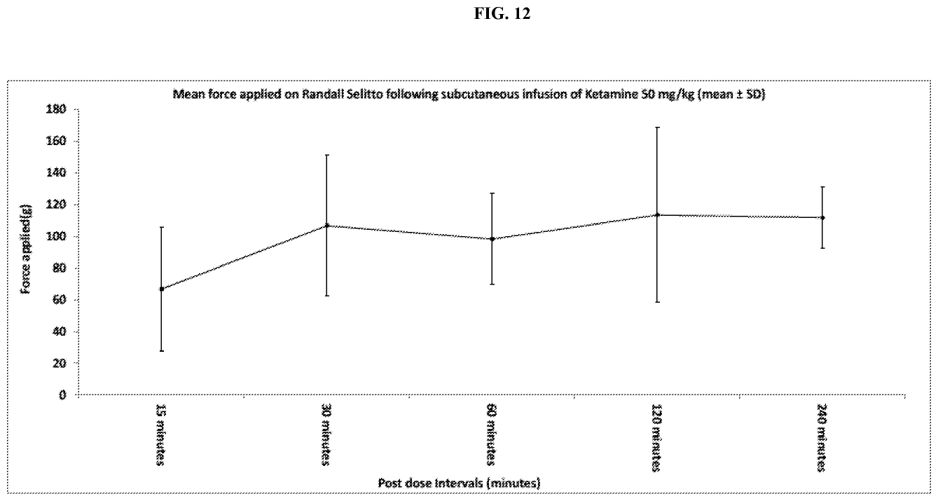

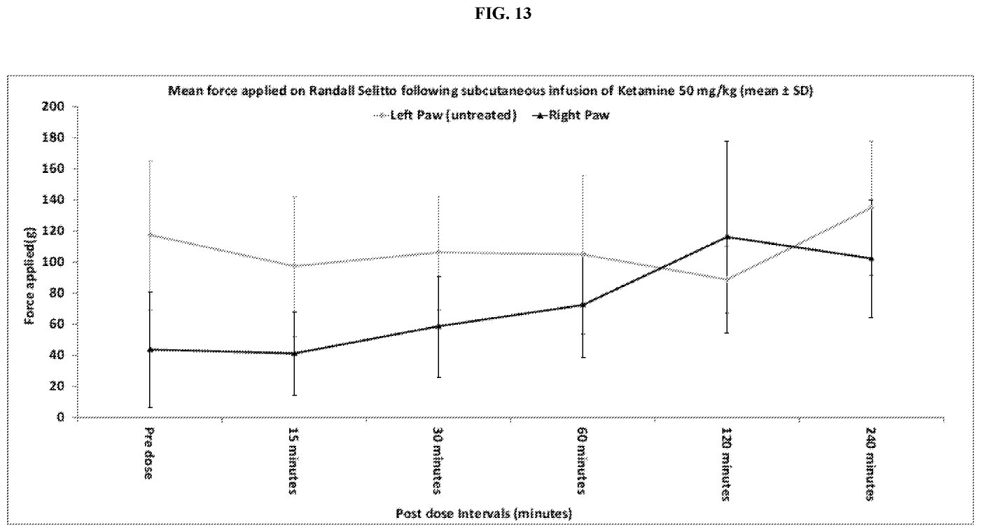

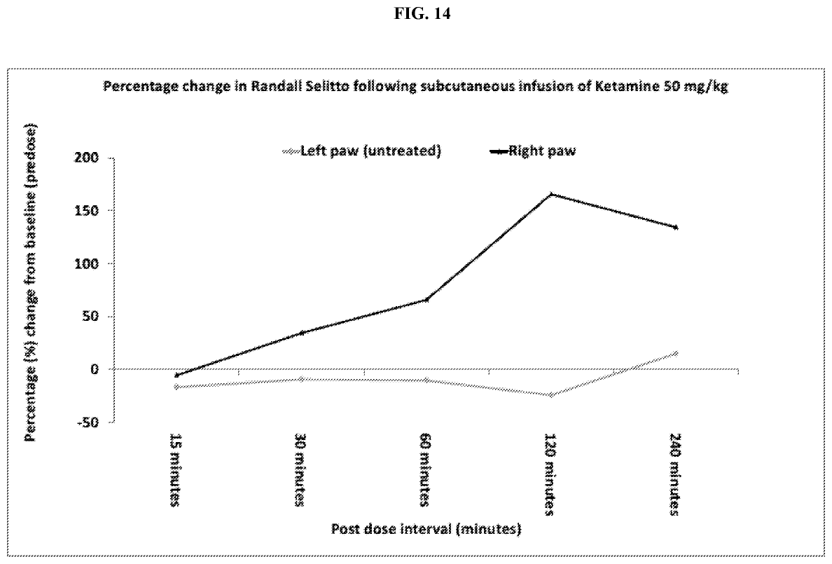

Results are summarized in Table 3 and Table 4; individual animal data are presented in Table 7 and Table 8. The data are graphically represented in FIG. 12, FIG. 13 and FIG. 14.

The omission of a pre-dose assessment following induction of a hyperalgesia and immediately prior to administration of ketamine made the interpretation of Group 1 data challenging. However, assuming the 15 minute time point was most closely reflective of the anticipated response immediately prior to ketamine administration, an increase in pressure required to initiate a response between 0.5 and 2 hours post-dose was noted for the majority of animals, which possibly suggested an analgesic effect.

Additional investigation (Group 4) demonstrated an increase in pressure required to initiate a response in the treated paw (right paw), in a time dependent manner, between 0.5 and 2 hours post-dose when compared to pre-dose. A slight decrease at 15 minutes post-dose was observed indicating an increase in sensitivity to the force applied. The force applied to the untreated paw (left paw) remained comparable to pre-dose throughout the data collection, suggesting that ketamine produced greater analgesia at sites of inflammation due to peripheral action.

TABLE 3
Summary of Randall Selitto

| | Force (g) applied to the inflamed (right) paw at time | | | | |
|---|---|---|---|---|---|
| | (h) post administration of ketamine (50 mg/kg, sc) | | | | |
| | 0.25 | 0.5 | 1 | 2 | 4 |
| Mean | 153.3 | 66.7 | 106.7 | 98.3 | 113.3 |
| SD | 76.07 | 38.82 | 44.57 | 28.58 | 55.02 |

Mean data for Randall Selitto (n=6, represented as mean±standard deviation SD)

TABLE 4
Summary of Randall Selitto Group 4

| | Pre-dose | 0.25 | 0.5 | 1 | 2 | 4 |
|---|---|---|---|---|---|---|
| | Force (g) applied to the inflamed (right) paw at time | | | | | |
| | (h) post administration of ketamine (50 mg/kg, sc) | | | | | |
| Mean | 43.8 | 41.3 | 58.8 | 72.5 | 116.3 | 102.5 |
| SD | 37.39 | 26.96 | 32.71 | 33.70 | 62.09 | 38.45 |
| % change | — | −5.7 | 34.3 | 65.7 | 165.7 | 38.45 |
| from | | | | | | |
| baseline | | | | | | |
| | Force (g) applied to the untreated (left) paw at time | | | | | |
| | (h) post administration of ketamine (50 mg/kg, sc) | | | | | |
| Mean | 117.5 | 97.5 | 106.3 | 105.0 | 88.8 | 135.0 |
| SD | 48.03 | 45.28 | 36.62 | 50.99 | 21.67 | 43.42 |
| % change | — | −17.0 | −9.6 | −10.6 | −24.5 | 14.9 |
| from | | | | | | |
| baseline | | | | | | |

Mean data for Randall Selitto (n=8, represented as mean±standard deviation SD)

TABLE 5
Summary of Writhing test (Abdominal Spasm)

| | Number of acetic acid -induced Abdominal |
|---|---|
| | Spasms at 1 hour following administration |
| | of ketamine (50 mg/kg, sc) |
| Mean | 4 |
| SD | 2.9 |

Mean data for writhing test (n=6, represented as mean±standard deviation SD)

TABLE 6
Individual tail flick data

| | Time (s) taken for response at time (h) | | | | | |
|---|---|---|---|---|---|---|
| Animal | post administration of ketamine (50 mg/kg, sc) | | | | | |
| number | Pre-dose | 0.25 | 0.5 | 1 | 2 | 4 |
| R0101 | 4.1 | 3.5 | 5.8 | 6.4 | 4.3 | 4.2 |
| R0102 | 5.6 | 6.5 | 5.8 | 5.8 | 4.9 | 6.2 |
| R0103 | 4.6 | 6.1 | 5.7 | 7.6 | 7.4 | 5.2 |
| R0104 | 5.2 | 4.2 | 5.2 | 5.9 | 5.2 | 5.1 |
| R0105 | 5.1 | 7.7 | 6.2 | 4.4 | 7.0 | 7.5 |
| R0106 | 6.3 | 5.5 | 6.4 | 7.4 | 6.5 | 7.4 |

TABLE 7
Individual Randall Selitto data

| | Force (g) applied to the inflamed (right) paw at time(h) | | | | |
|---|---|---|---|---|---|
| Animal | post administration of ketamine (50 mg/kg, sc) | | | | |
| Number | 0.25 | 0.5 | 1 | 2 | 4 |
| R0001 | 120 | 170 | 60 | 200 | 140 |
| R0002 | 90 | 120 | 120 | 120 | 90 |
| R0003 | 40 | 40 | 80 | 140 | 90 |
| R0004 | 90 | 100 | 130 | 60 | 120 |
| R0005 | 20 | 130 | 120 | 110 | 120 |
| R0006 | 40 | 80 | 80 | 50 | 110 |

TABLE 8
Individual Randall Selitto data Group 4

| | Force (g) applied to the inflamed (right) paw at time | | | | | | | | | | | |
|---|---|---|---|---|---|---|---|---|---|---|---|---|
| | (h) post administration of ketamine (50 mg/kg, sc) | | | | | | | | | | | |
| | Pre dose | | 0.25 | | 0.5 | | 1 | | 2 | | 4 | |
| Animal | Left | Right | Left | Right | Left | Right | Left | Right | Left | Right | Left | Right |
| Number | paw | Paw | paw | Paw | paw | Paw | paw | Paw | paw | Paw | Paw | Paw |
| R0001 | 80 | 20 | 30 | 0 | 40 | 0 | 40 | 30 | 60 | 70 | 130 | 70 |
| R0002 | 110 | 30 | 60 | 10 | 120 | 40 | 80 | 80 | 100 | 180 | 130 | 60 |
| R0003 | 100 | 80 | 100 | 30 | 130 | 50 | 80 | 60 | 70 | 80 | 110 | 90 |
| R0004 | 170 | 20 | 180 | 80 | 150 | 80 | 160 | 140 | 130 | 240 | 220 | 170 |
| R0005 | 190 | 10 | 90 | 50 | 120 | 90 | 100 | 40 | 80 | 70 | 110 | 100 |
| R0006 | 90 | 40 | 80 | 40 | 90 | 70 | 80 | 70 | 80 | 80 | 90 | 80 |
| R0007 | 50 | 30 | 130 | 60 | 70 | 40 | 100 | 70 | 90 | 120 | 110 | 100 |
| R0008 | 150 | 120 | 110 | 60 | 130 | 100 | 200 | 90 | 100 | 90 | 180 | 150 |

TABLE 9
Individual writhing test data

| | Number of acetic acid -induced Abdominal |
|---|---|
| | Spasms at 1 hour following administration |
| Animal Number | of ketamine (50 mg/kg, sc) |
| R0201 | 5 |
| R0202 | 1 |
| R0203 | 8 |
| R0204 | 0 |
| R0205 | 4 |
| R0206 | 3 |

2.4 Abdominal Spasm (Writhing) Test

The results are summarized in Table 4; individual animal data are presented in Table 8.

In this study, intraperitoneal administration of 1% acetic acid to control animals produced a marked irritant effect, with a group mean of 29 abdominal spasms within a 25-minute observation period.

Subcutaneous administration of 50 mg/kg ketamine produced a group mean of four abdominal spasms within a 25 minute observation period following intraperitoneal administration of acetic acid, which suggested a marked analgesic response. However, due to clinical observations noted prior to administration of the irritant, an anesthetic affect could not be ruled out. Assessment at later post-dose time points is recommended in order to further characterize this response.

### 3. Conclusion

Pain is a complex experience that can be classified into a number of types of modalities depending on the triggering stimulus of pain. Translation of preclinical research models of nociception to pain treatment in the clinic has been met with difficulties (Deuis et al., 2017). An understanding that the human pain experience encompasses multiple stimulus modalities, molecular mechanisms, and sensory and motor components highlights the need for carefully designed experiments that take the complexity of pain in humans into consideration.

The objective of this study was to select a pain model for which ketamine demonstrated efficacy, and, as such, the tests were designed to detect gross effects using a limited number of animals, with comparisons with background data and/or pre-dose values.

Preliminary and limited investigations of the analgesic effect of 50 mg/kg ketamine administered via an infusion pump demonstrated possible analgesia in all three selected models, in particular against peripherally acting pain and hyperalgesia.

In order to confirm these effects, further investigations are required using complete study designs that include control animals and multiple dose levels to determine dose response effects. While the appropriate methods for further investigation should be considered, a greater understanding of pain modulation in the targeted clinical conditions is required.

### 4. Abbreviations

The following lists of codes, abbreviations, and comments on the data are used in this report.
% RSD Relative standard deviation
CAM Covariate-adjusted mean
CV Coefficient of variation
F Female
H, h Hour
HR Heart rate
ID Identification
KG, kg Kilogram
M Male
Mean Arithmetic mean
Mg, mg Milligram
mL Milliliter
Msec, msec Milliseconds
n Number of animals/measurements in a group
N/A, n/a Not applicable
NAD No abnormalities detected
P(DR) P value (dose response)
P(overall) Overall P value for all groups
P(v1) P value (verses group 1)
PD Post-dose
PT Pre-treatment
S, s, sec Seconds
SD Standard deviation
S.E.M./SEM Standard error mean
LOQ Limit of quantification
Percent difference
% RSD Relative standard deviation
CAM Covariate-adjusted mean
CV Coefficient of variation

### Example 8—Minipig Pharmacokinetics Study (BB-1802)

Objective

The purpose of this study was to determine the pharmacokinetics of ketamine after a single subcutaneous infusion dose to minipigs.

Test Article

| | Number/ | | | Target Total | Target Dose | Target Dose | Target Dose |
|---|---|---|---|---|---|---|---|
| Phase/ | Sex of | Test | Dose | Dose Level | Level/Hour | Concentration | Volume |
| Group | Animals | Article | Route | (mg/kg) | (mg/kg/hr) | (mg/mL) | (mL/kg)^{a} |
| 1/1 | 2/M | Ketamine | SC^{b} | 4 | 0.5 | 100 | 0.04 |
| 2/1 | 2/M | Ketamine | SC^{b} | 8 | 1 | 100 | 0.08 |
| 3/1 | 2/M | Ketamine | SC^{b} | 12 | 1.5 | 100 | 0.12 |
| 4/1 | 1/F | Ketamine | SC^{c} | 18 | 1 | 100 | 0.18 |
| 5/1 | 1/F | Ketamine | SC^{c} | 18 | 1 | 100 | 0.18 |

F Female.

M Male.

SC Subcutaneous infusion via insulin pump.

Note: There was at least a 3-day washout period between phases.

a Total target dose volume delivered over the course of the infusion.

b Administered as an approximately 8-hour infusion.

c Administered as an approximately 18-hour infusion.

Animals and Husbandry

Strain and Source: Male Gottingen Minipigs from Marshall Farms were received on 26 Jul. 2018. The animals were acclimated to study conditions for 25 days prior to initial dose administration. An additional female Gottingen Minipig from Marshall Farms (previously jugular-vein cannulated) was transferred on 4 Sep. 2018. The animal was acclimated to study conditions for 9 days prior to initial dose administration. At initial dosing (Phase 1), the animals weighed 14.5 and 15.3 kg and were 6 to 8 months of age.

Housing: During acclimation and the test period, animals were housed in stainless steel cages.

Feed and Water: Certified Diet #5K99 (PMI, Inc.) was provided in accordance with Covance SOPs. Water was provided fresh daily, ad libitum.

Enrichment and Treats: For environmental and psychological enrichment, various cage and/or food enrichment (that did not require analysis) were offered in accordance with the applicable SOPs. Diets were supplemented with appropriate treats (that did not require analysis) in accordance with Covance SOPs.

Environment: Environmental controls for the animal room were set to maintain a temperature of 20 to 26° C., a relative humidity of 50±20%, and a 12-hour light/12-hour dark cycle. As necessary, the 12-hour dark cycle was interrupted to accommodate study procedures.

Surgical Requirements: All male animals had a physical examination conducted by a veterinarian or trained veterinary technician prior to surgery procedures. All male animals were anesthetized and a jugular vein catheter was inserted by surgical procedure; the procedure was followed by at least 14 days of recovery before administration of the test article.

Animal Selection: Animals were not randomized. Animals were selected for use on test based on overall health and cannula patency.

Identification: Animals were identified via individual cage cards and implantable microchip identification devices (IMID).

Dose Preparation and Analysis: The test article was purchased from a commercially available source and used as supplied. The test article appeared as a clear, colorless solution. Any dose formulation remaining following administration was stored at ambient temperature.

Dose Procedures: Individual doses were calculated based on body weights recorded on each day of dose administration.

Fasting: Animals were not fasted.

Dose Administration: In each phase, animals received a single subcutaneous infusion (up to 18 hours) via insulin pump (Medtronic MiniMed Paradigm® Insulin Pump). The infusion pump was programmed in accordance with a Study Specific Procedure, and the infusion was continuously monitored for the duration of the dose. In Phase 3, the pump for Animal P0002 (Group 1 male) alarmed at approximately 1 hour postdose. The pump was inspected, re-secured to the animal, and the dose continued.

Observation of Animals

Ante mortem Observations: On the day of arrival or transfer, animals were observed for mortality and signs of pain and distress once (p.m.), and cage side observations were done for general health and appearance. Beginning the day after arrival or transfer, animals were observed for mortality and signs of pain and distress twice daily (a.m. and p.m.), and cage side observations for general health and appearance were done once daily.

Body Weights: Body weights of the male animals were taken within 5 days of arrival. Body weights of all animals were taken weekly throughout acclimation, as applicable. Animals were also weighed at the time of animal selection and on the day of each dose administration, as applicable. Additional body weights were taken throughout the study for monitoring.

Sample Collection

For each phase, blood (approximately 2 mL) was collected via jugular vein catheter into tubes containing K3EDTA from each animal predose and at approximately 0.083, 0.25, 0.5, 1, 2, 4, 8, 9 (Phases 1 through 3 only), 10 (Phases lthrough 3 only), 12, 15 (Phases 4 and 5 only), 18 (Phases 4 and 5 only), 20 (Phases 4 and 5 only), 22 (Phases 4 and 5 only), and 24 hours postdose, with the following exceptions in Phase 3 for Animal 50004 (Group 1 male). Blood was collected at the 1-hour time point via the anterior vena cava, the 2-hour blood sample was a short sample of approximately 1 mL (Deviation), and the 12-hour blood sample could not be obtained (Deviation). Times were based on the start of the infusion.

Blood was maintained in chilled cryoracks prior to centrifugation to obtain plasma. Centrifugation began within 1 hour of collection, with the following exception. In Phase 2, the centrifugation times for the 0.083- and 0.25-hour samples for Animal 50003 (Group 1 male) were inadvertently not recorded and cannot be verified (Deviation). Plasma was placed into 96-well tubes with barcode labels. Plasma was maintained on dry ice prior to storage at approximately −70° C.

The 9-hour plasma sample in Phase 3 for Animal 50004 (Group 1 male) and the 2-hour plasma sample in Phase 5 for Animal 50005 (Group 1 female) appeared red in color.

Plasma samples were analyzed for concentrations of ketamine using an established liquid chromatography/mass spectrometry (LC-MS/MS) method.

Data Analysis

Noncompartmental analysis (1) was applied to the individual plasma ketamine concentration data for males and females. The following parameters were estimated whenever possible:

| Parameters | Definition |
|---|---|
| Cₘₐₓ | Maximum observed concentration. |
| Tₘₐₓ | Time of maximum observed concentration. |
| Cₘᵢₙ | Minimum measureable (non-zero) concentration. |
| Tₘᵢₙ | Time of minimum measureable (non-zero) concentration. |
| Cₛₛ | Concentration at steady-state, calculated based on visual |
| | inspection of the plasma concentration-time profiles, |
| | assuming a constant rate of absorption. |
| Tₛₛ | Time to steady-state, calculated based on visual inspection |
| | of the plasma concentration-time profiles, assuming a |
| | constant rate of absorption. |
| AUC₀₋ₜ | Area under the concentration-time curve from hour 0 to the |
| | last measurable concentration, estimated by the linear |
| | trapezoidal rule. |
| AUC₀₋₂₄ | Area under the concentration-time curve from hour 0 to |
| | hour 24, estimated by the linear trapezoidal rule. |
| AUC_{0-∞} | Area under the concentration-time curve from hour 0 to |
| | infinity for Day 1, calculated as follows: |

AUC₀₋_{^{∞}}=AUC₀₋ₜ+Cₜ/λ_{z}

Where Cₜ is the last measurable concentration and λ_{z} is the elimination rate constant estimated using log-linear regression during the terminal elimination phase. The number of points used in λ_{z} calculation was determined by visual inspection of the data describing the terminal phase. At least the last three time points with measurable values were used in λ_{z} calculation.

| | |
|---|---|
| t_{1/2} | Elimination half-life, determined by ln(2)/λ_{z}. |
| DN Cₘₐₓ | Dose normalized Cₘₐₓ, calculated as Cₘₐₓ/dose level. |
| DN AUC₀₋₂₄ | Dose normalized AUC₀₋₂₄, calculated as AUC₀₋₂₄/dose |
| | level |

Nominal doses and sampling times were used. Concentration values below the lower limit of quantitation (<10.0 ng/mL) were treated as zero for descriptive statistics and pharmacokinetic analysis. Embedded zeros were excluded from pharmacokinetic analysis.

Because the data were computer-generated and rounded appropriately for inclusion in the report, the use of reported values to calculate subsequent parameters will, in some instances, yield minor variations from those listed in the tables. Neither the integrity nor the interpretation of the data were affected by these differences.

Unexpected Results Relevant to Data Analysis

The following blood sample was unable to be obtained. There is no impact on PK evaluation due to this deviation as Cₘₐₓ was generally around 4.00 hours for this animal.

| | | Dose Level | | Animal | Nominal |
|---|---|---|---|---|---|
| Phase | Dose Group | (mg/kg) | Sex | Number | Time (hr) |
| 3 | 1 | 8 | M | S0004 | 12 |

Animals 50001, 50004 (Group 1 males) were euthanized in moribund condition on Day 8 of Phases 2 and 3, respectively. There is no impact on PK evaluation due to this event to Phases 2 or 3, as full profiles were collected for these animals. Due to these early deaths, Animal S0005 (Group 1 female) was added as a replacement to complete Phases 4 and 5.

During Phase 1, Animal 50002 (Group 1 male) did not receive the complete dose and only received approximately 30% of the intended dose. Due to this incomplete dose, Animal S0002 (4 mg/kg) was excluded from pharmacokinetic evaluation and descriptive statistics.

Results

Body Weights and Dose Administration

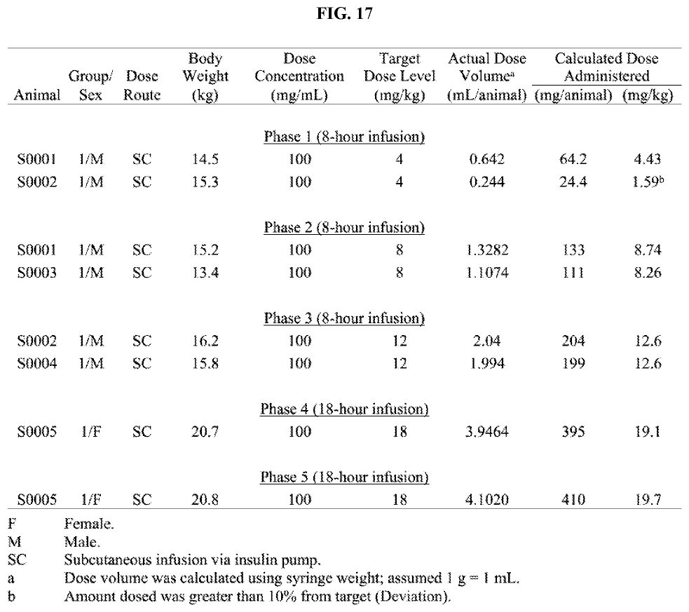

Individual animal body weights and doses administered are presented in FIG. 17. The amount of ketamine dosed to Animal S0002 (Group 1 male) in Phase 1 was 1.59 mg/kg, which was greater than 10% from the target of 4 mg/kg (Deviation).

Sample Collections

According to the time ranges below, all collections were made within the acceptable ranges. A summary of acceptable time ranges follows.

| | Acceptable |
|---|---|
| Scheduled | Deviation from |
| Collection Time | Scheduled Time |
| 0-15 minutes | ±1 minute |
| 16-30 minutes | ±2 minutes |
| 31-45 minutes | ±3 minutes |
| 46-60 minutes | ±4 minutes |
| 61 minutes-2 hours | ±5 minutes |
| 2 hours 1 minute-8 hours | ±10 minutes |
| >8 hours-24 hours | ±20 minutes |
| >24 hours | ±60 minutes |

Animal Observations

All animals appeared healthy prior to dosing and throughout the duration of the study, with the following observations noted.

| | | Phase/Study | |
|---|---|---|---|
| | | Day of | |
| Group/Sex | Animals | Observation | Observation |
| 1/Male | S0001a | Phase 1/Day 2 | Stitches broken at the dorsal |
| | | | cervical catheter exit site |
| | | Phase 1/Day 3 | Bright, alert, responsive, |
| | | | sutures replaced |
| | | Phase 2/Day 8 | Twitching over entire body, |
| | | | animal was euthanized |
| 1/Male | S0002 | Phase 1/Day 2 | Stitches broken at the dorsal |
| | | | cervical catheter exit site |
| | | Phase 1/Day 3 | Bright, alert, responsive, |
| | | | sutures replaced |
| 1/Male | S0003 | Phase 2/Day 2 | Stitches broken at the dorsal |
| | | | cervical catheter exit site |
| | | Phase 2/Day 3 | Bright, alert, responsive, |
| | | | sutures replaced |
| 1/Male | S0004b | Phase 3/Day 8 | Bright, limited use of hind legs/ |
| | | | weak hind legs, ataxic, discolored |
| | | | purple ears and midline dorsal |
| | | | thorax, entire body pale, formed |
| | | | feces, ate all feed, general body |
| | | | condition normal, large firm area |
| | | | of red to dark purple skin on the |
| | | | left lateral neck and generalized |
| | | | small patches of bruising (red) on |
| | | | body, legs, and ears, jugular |
| | | | catheter not patent, body temper- |
| | | | ature was normal (39.1° C.), |
| | | | animal was euthanized per veteri- |
| | | | nary directive for humane concern. |

a A necropsy was performed on Animal S0001 (Group 1 male) on the day of euthanasia. Skin/subcutis had a raised area, ventral abdomen, clear fluid on the cut surface, up to 5 mm. All lobes of the lungs were discolored, multiple dark red areas up to 10 mm. Adverse effects were considered to be related to the surgically placed indwelling catheter and not test article related.

b A necropsy was performed on Animal 50004 (Group 1 male) on the day of euthanasia. Skin/subcutis had multiple discolored dark red areas over the entire body up to 20 mm. The entire bilateral inguinal lymph node was discolored dark red. The jejunum had multiple pinpoint discolored red serosa in the mid-section. Adverse effects were considered to be related to the surgically placed indwelling catheter and not test article related.

Concentrations of Test Article

Plasma Concentrations

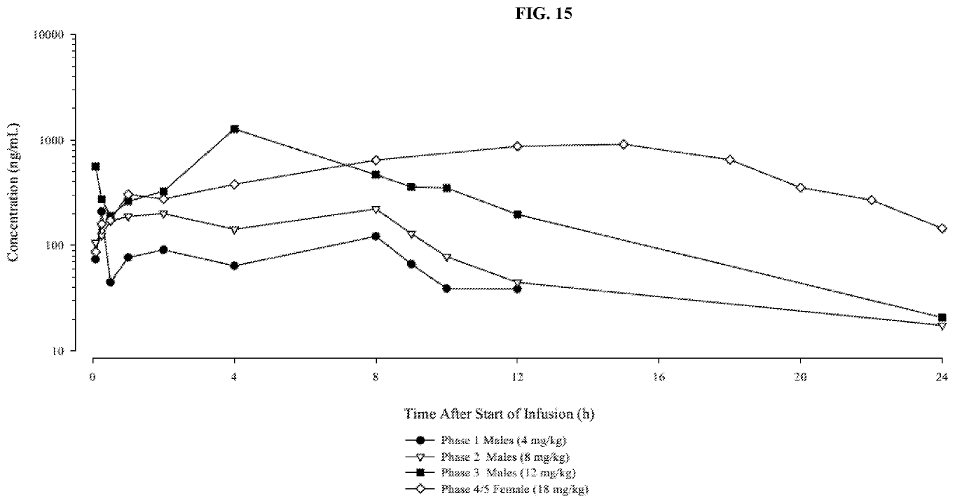

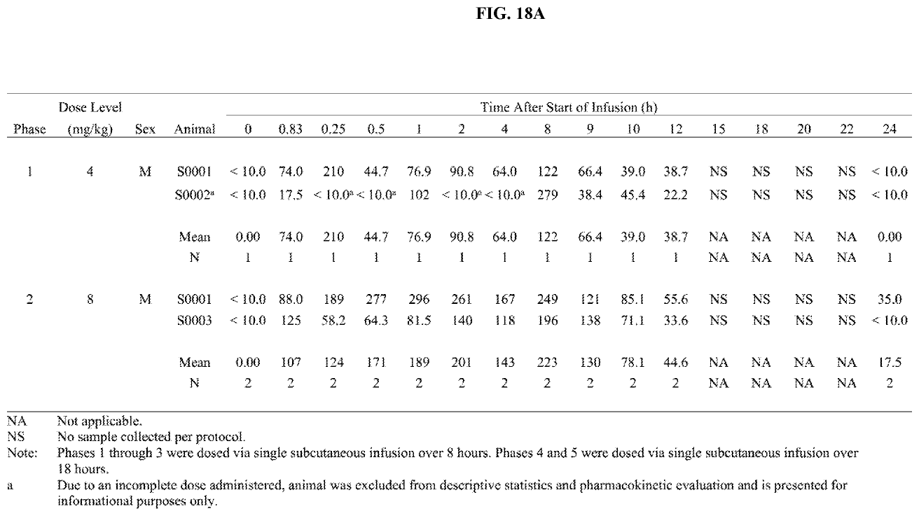

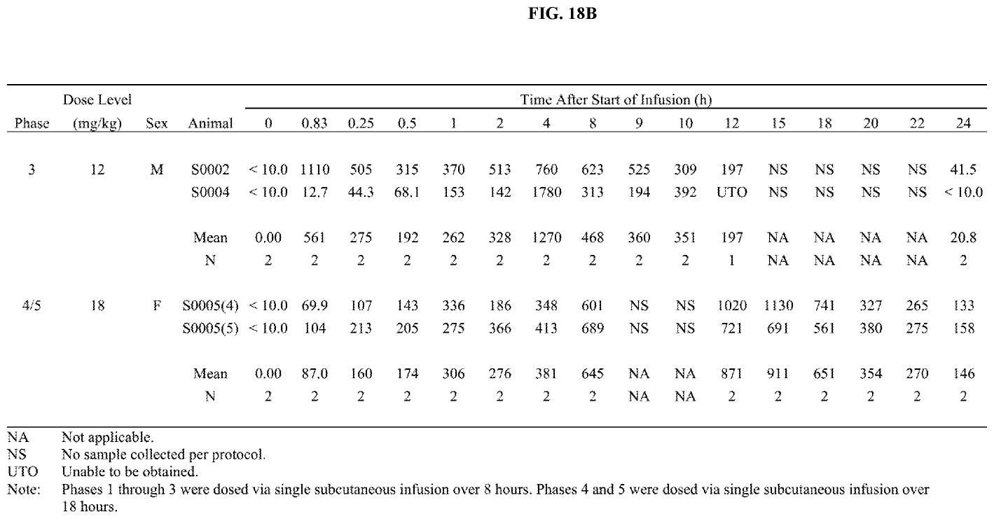

The individual and mean concentrations of ketamine in pig plasma are presented in FIGS. 18A-18B. The mean concentration-time profiles of ketamine in pig plasma are presented graphically in FIG. 15.

Pharmacokinetic Parameters

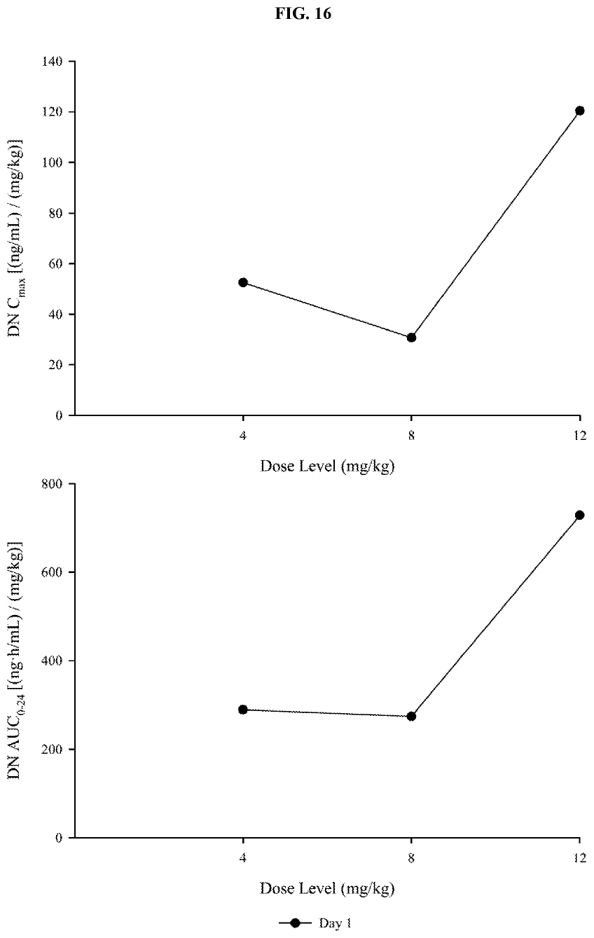

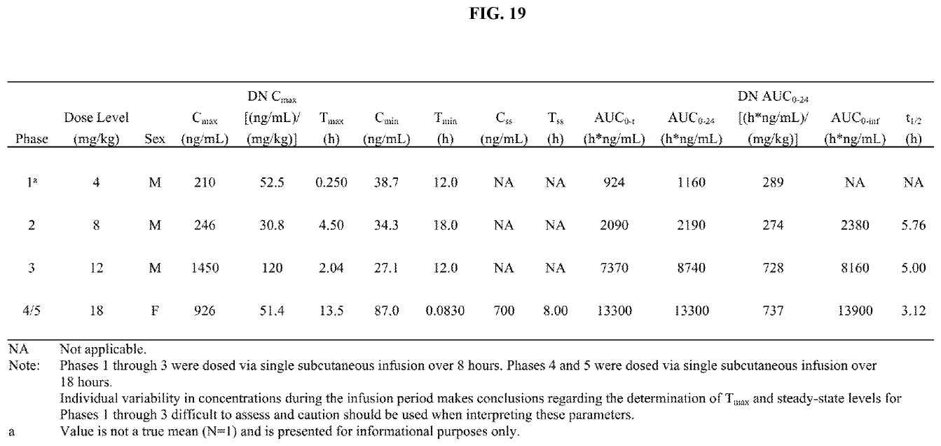

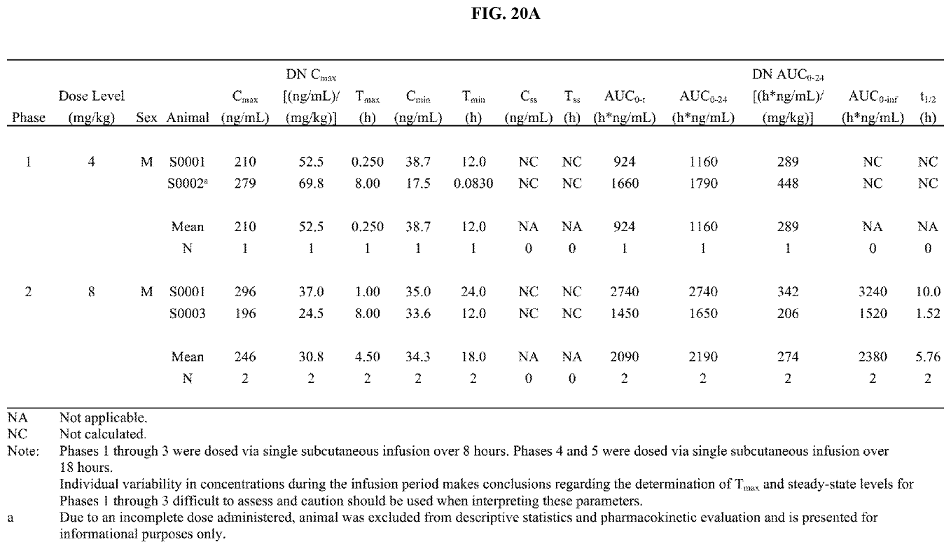

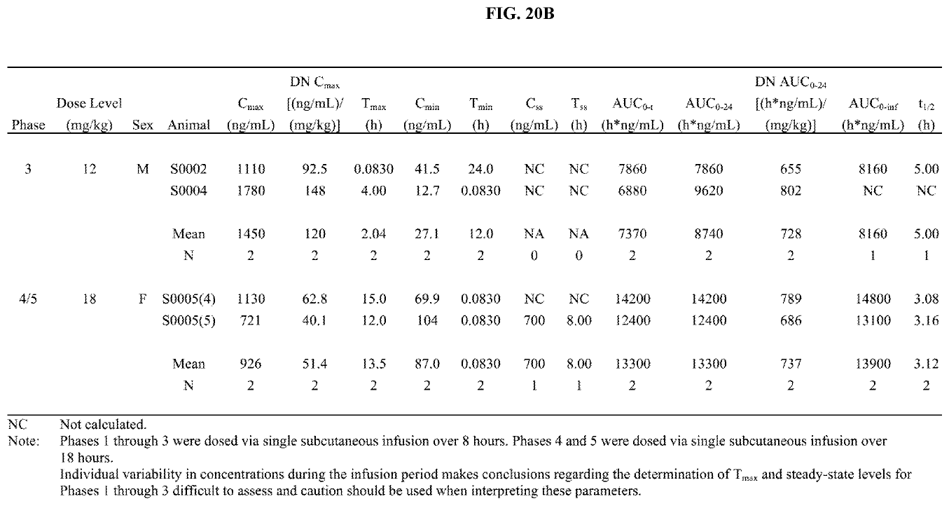

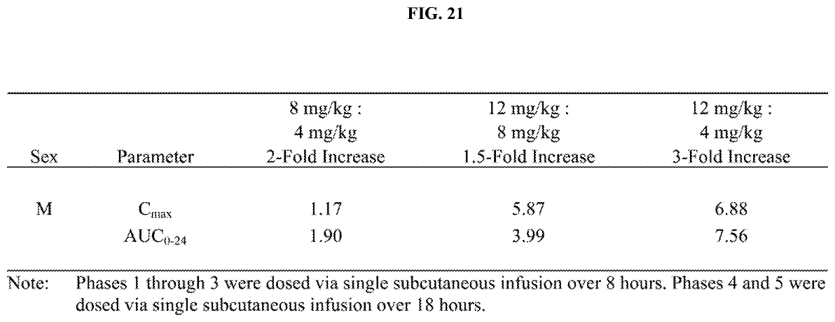

The summary of the mean pharmacokinetic parameters of ketamine in pig plasma are presented in FIG. 19. The individual and mean pharmacokinetic parameters of ketamine in pig plasma are presented in FIGS. 20A-20B. The dose proportionality ratios for ketamine Cₘₐₓ and AUC₀₋₂₄ in pig plasma are presented in FIG. 21. The dose normalized Cₘₐₓ and AUC₀₋₂₄ relationships of ketamine in pig plasma are presented graphically in FIG. 16.

Pharmacokinetic Profile

After a single subcutaneous infusion administration over 8 hours for Phases 1 through 3 and 18 hours for Phases 4 and 5, ketamine was absorbed, with mean Tₘₐₓ values at 0.250 hours for Phase 1, 4.50 hours for Phase 2, 2.04 for Phase 3, and at 13.5 for Phases 4 and 5. Concentrations in Phases 1 through 4 did not appear to reach a steady-state; however, concentrations in Phase 5 appeared to reach steady-state from approximately 8.00 to 15.0 hours, with a mean Cₛₛ value of 700 ng/mL. After end of infusion, ketamine concentrations declined, with the mean t_{1/2} values at 5.76 for Phase 2, at 5.00 for Phase 3, and at 3.12 for Phases 4 and 5. Due to the lack of a distinct elimination phase, estimation of elimination phase half-life (t½) was not attempted for animals in Phase 1. Mean concentration values for ketamine were measurable through 12 hours post the start of infusion for Phase 1 and through 24 hours post the start of infusion for Phases 3 through 5.

The mean concentration-time profiles for males and females (FIG. 17) show that exposure to ketamine generally increased with the increase in dose level from 4 to 18 mg/kg.

Dose Proportionality

The exposure, as assessed by ketamine mean Cₘₐₓ and AUC₀₋₂₄ values, generally increased with the increase in dose level from 4 to 12 mg/kg. The increases in ketamine mean Cₘₐₓ and AUC₀₋₂₄ values for males were less than dose proportional with an increase in dose level from 4 to 8 mg/kg and greater than dose proportional with a further increase in dose level from 8 to 12 mg/kg.

Pharmacokinetic Conclusions

Exposure to ketamine generally increased with the increase in dose level from 4 to 18 mg/kg.

Concentrations in Phases 1 through 4 did not appear to reach a steady-state; however, concentrations in Phase 5 appeared to reach steady-state from approximately 8.00 to 15.0 hours, with a mean Cₛₛ value of 700 ng/mL.

The increases in ketamine mean Cₘₐₓ and AUC₀₋₂₄ values for males were less than dose proportional with an increase in dose level from 4 to 8 mg/kg and greater than dose proportional with a further increase in dose level from 8 to 12 mg/kg.

### Example 9—Animal Studies Analysis

Ketamine is an NMDA receptor antagonist with a wide variety of clinical effects. A short half-life and high first-pass metabolism decreases bioavailability of ketamine via non-IV or IM ROAs low and increases the ratio of non-active to active metabolites, increasing risk of bladder cystitis as the primary end elimination organ in detoxification. Described herein are two pilot studies from examples 7 and 8 examining the viability of the potential efficacy and pharmacokinetic innovation with continuous subcutaneous delivery of ketamine via a wearable pump device as a treatment mode for pain management.

In the first study, BB-1802, subcutaneous ketamine was delivered at 1 mg/kg/hr to minipigs with a personal insulin pump produced a steady-state from approximately 8.00 to 15.0 hours, with a mean Cₛₛ value of 700 ng/mL. This validated that ketamine can be delivered subcutaneously through dermal tissue similar to humans and can achieve targeted blood levels within clinically relevant time frames.

In the second pilot experiment, BB-1803, three pain models in rats were explored with subanesthetic infusion with an insulin pump. Results with the tail flick pain model revealed a smooth curve in analgesic effects from 15 minutes through the final data gathered at 4 hours. The maximum effect was a 21.4% increase in time to tail flick. The Randall-Selitto test demonstrated an increase in pressure required to initiate a response in the treated paw (right paw), in a time dependent manner, between 0.5 and 2 hours post-dose when compared to pre-dose. The force applied to the untreated paw (left paw) remained comparable to pre-dose throughout the data collection, suggesting that ketamine produced greater analgesia at sites of inflammation due to peripheral action. In the abdominal spasm test, ketamine infusion reduced the number of elicited spasms from a baseline of 29/25 min to an average of 4/25 min, an 86% reduction from baseline values. Thus, delivery of subcutaneous ketamine by insulin pump in these pilot studies achieved a mean steady-state from approximately 8.00 to 15.0 hours, with a mean Cₛₛ value of 700 ng/mL, and demonstrated significant pain relief in three different pain models, namely the rat tail flick test, the Randall-Selitto test, and abdominal spasm tests.

BB-1802 Mini-Pig—8 and 16 Hour Infusions:

After a single subcutaneous infusion administration over 8 hours for Phases 1 through 3 and 18 hours for Phases 4 and 5, ketamine was absorbed, with mean Tₘₐₓ values at 4.13 hours for Phase 1, 4.50 hours for Phase 2, 2.04 for Phase 3, and at 13.5 for Phases 4 and 5. Concentrations in Phases 1 through 4 did not appear to reach a steady-state; however, concentrations in Phase 5 appeared to reach steady-state from approximately 8.00 to 15.0 hours, with a mean Cₛₛ value of 700 ng/mL. After end of infusion, ketamine concentrations declined, with the mean t_{1/2} values at 5.76 for Phase 2, at 5.00 for Phase 3 and at 3.12 for Phases 4 and 5. Due to the lack of a distinct elimination phase, estimation of elimination phase half-life (t_{1/2}) was not attempted for animals in Phase 1. Mean concentration values for ketamine were measurable through 12 hours post the start of infusion for Phase 1 and through 24 hours post the start of infusion for Phases 3 through 5. The mean concentration-time profiles for males and females show that exposure to ketamine generally increased with the increase in dose level from 4 to 18 mg/kg. The exposure, as assessed by ketamine mean Cₘₐₓ and AUC₀₋₂₄ values, generally increased with the increase in dose level from 4 to 12 mg/kg. The increases in ketamine mean Cₘₐₓ and AUC₀₋₂₄ values for males were less than dose proportional with an increase in dose level from 4 to 8 mg/kg and greater than dose proportional with a further increase in dose level from 8 to 12 mg/kg. Exposure to ketamine generally increased with the increase in dose level from 4 to 18 mg/kg. In all animals, plasma ketamine levels were detectible within 5 minutes of initiating subcutaneous infusion. In most animals, initial spikes in plasma levels were seen in the first 15 to 60 minutes but trended downward by hour two, likely reflecting the beginning of equilibrium between local redistribution of subcutaneous ketamine to the plasma versus redistribution of ketamine from plasma to whole body tissues. After this short period of dropping plasma levels from 60 to 120 minutes a shallow upward slope in plasma levels occurred. The same animal was used for both 18-hour infusions, separated by 3 days to allow for recovery.

BB-1803 Rat Study:

The objective of this study was to select a pain model for which ketamine demonstrated efficacy, and, as such, the tests were designed to detect gross effects using a limited number of animals, with comparisons with background data and/or pre-dose values. Animal models of pain have two important components: the method of injury and end-point measurement. These models can be divided into stimulus evoked (mechanical, heat or cold, or irritant) and non-stimulus evoked. For the purposes of this study, evoked models were used, including tail flick, Randall-Selitto, and abdominal spasm (writhing test) tests.

The tail flick test involves application of a heat stimulus (infrared light) to the tail, and latency to withdrawal from the stimulus is recorded. The response measured is immediate, uses the Aδ- and C-fiber inputs, and is known to activate the spinal dorsal horn, the cells of which are nociceptive-specific. The response has been reported as proportional to the frequency of stimulus and the fiber class of afferent input. However, a similar response has been observed in spinally transected rats, indicating that the tail withdrawal response is a spinal reflex, rather than a pain behavior involved in higher brain centers. Conversely, the contribution of supraspinal processing to the tail flick response is dependent on the heating slope of the stimulus that leads to more delayed responses involving higher central nervous system functions believed to be necessary to process pain. Tail-flick tests have been reliably used for determining the potency of opioid analgesics and, as such, can prove valuable for predicting analgesic effects in humans. Subcutaneous infusion of 50 mg/kg ketamine produced a slight increase in latency of tail flick at all post-dose time points. The group mean reaction time was longest at 1 hour post-dose (6.3 seconds; a 21.4% increase from baseline [5.2 seconds]). The shortest reaction time was elicited at 15 minutes post-dose (5.6 seconds; an 8.4% increase from baseline). Although the time taken for the sensation of pain (induced by infrared beam) to have been perceived was increased in a time dependent manner, this was of small magnitude (1.1 second) and, as such, was not conclusive of an analgesic response.

The Randall-Selitto test is a tool to assess the response thresholds to mechanical pressure stimulation following induction of a hyperalgesic state by injection of an inflammatory agent and is considered a measure of mechanical hyperalgesia. This involves application of an increasing force to the surface of the paw until withdrawal or vocalization occurs. The test can produce results similar to decreases on pressure pain thresholds observed in clinical conditions, such as fibromyalgia, myofascial pain, or osteoarthritis. Centrally and peripherally acting effects can be detected using this model, for example, opioids increase the mechanical threshold to the normal and inflamed paw, whereas nonsteroidal anti-inflammatory drugs are only effective in the inflamed paw. Rats tested demonstrated an increase in pressure required to initiate a response in the treated paw (right paw), in a time dependent manner, between 0.5 and 2 hours post-dose when compared to pre-dose. A slight decrease at 15 minutes post-dose was observed indicating an increase in sensitivity to the force applied. The force applied to the untreated paw (left paw) remained comparable to pre-dose throughout the data collection, suggesting that ketamine produced greater analgesia at sites of inflammation due to peripheral action.

In the abdominal spasm (acetic acid-induced writhing) test, acetic acid is injected into the peritoneal cavity where it directly activates nociceptors and results in an abdominal writhing response. This writhing response is characterized by contraction of abdominal muscles followed by an extension of the hind limbs. This pain-behavior is considered reflexive and evidence of visceral pain associated with visceral chemoreceptors. However, the test lacks specificity as it has been shown to work well for all major and minor analgesics. It is widely accepted that this model can be used as a screening method to determine the effects of drugs on inflammatory induced nociception. In this study, intraperitoneal administration of 1% acetic acid to control animals produced a marked irritant effect, with a group mean of 29 abdominal spasms within a 25-minute observation period. Subcutaneous administration of 50 mg/kg ketamine produced a group mean of four abdominal spasms within a 25-minute observation period following intraperitoneal administration of acetic acid, which suggested a marked analgesic response.

These multi-phase, multi-animal pilot studies demonstrated a number of important facts. In BB-1802, subcutaneous ketamine delivery by insulin pump produced sometimes substantial anti-pain effects in three different pain models/tests in rats (tail flick, Randall-Selitto and abdominal spasm). The BB-1802 mini-pig study demonstrated that an insulin pump can safely deliver ketamine subcutaneously to achieve measurable, reasonable and controllable serum levels over the course of hours, through a dermal layer similar to humans. In the shorter, 8-hour infusions, it was demonstrated that ketamine concentrations drop rapidly upon stopping infusion. This has positive implications for clinical use in humans, where the capacity for a rapid return to baseline can be desired in the use of ketamine specifically, and in pain management generally. This rapid return to baseline can allow more aggressive dosing regimens, secure in the knowledge that stopping infusion will reverse effects rapidly. It also confirms the clinical limitations associated with ketamine's short T₁₁₂ in any bolus delivery models.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A drug delivery device comprising:
a) a pump mechanism configured for administering a drug formulation comprising an NMDA receptor antagonist; and
b) a user interface allowing a subject to select and self-administer a dose of the drug formulation from a selection of at least one pre-programmed dosage regimen that is not configurable by the subject;
wherein the at least one dosage regimen provides an effective drug plasma concentration.

2. The drug delivery device of claim 1, wherein the at least one dosage regimen provides an effective steady state drug plasma concentration.

3. The drug delivery device of claim 1, wherein the at least one dosage regimen is locked after configuration by an authorized user to deter modification by the subject.

4. The drug delivery device of claim 1, wherein the at least one dosage regimen is locked after configuration by the manufacturer to deter modification by the patient.

5. The drug delivery device of claim 1, wherein the drug delivery device is configured to be tamper-resistant to deter administration of a dose of the drug formulation that deviates from the at least one dosage regimen.

6. The drug delivery device of claim 1, wherein the drug formulation is stored in tamper-resistant cartridge.

7. The drug delivery device of claim 1, wherein the drug delivery device comprises a reservoir for storing the drug formulation prior to administration.

8. The drug delivery device of claim 1, wherein the at least one dosage regimen reduces side effects of the drug formulation while providing the state drug plasma concentration.

9. The drug delivery device of claim 8, wherein the side effects comprise drug dependence or addiction.

10. The drug delivery device of claim 8, wherein the side effects comprise hallucination, disorientation, dissociation, dizziness, drowsiness, increased heart rate, elevated blood pressure, nausea, vomiting, fatigue, brain fog, confusion, anxiety, distress, shortness of breath, or any combination thereof.

11. The drug delivery device of claim 1, wherein the drug delivery device is configured to administer the drug formulation according to the at least one dosage regimen for treating major depressive disorder, treatment resistant major depressive disorder, suicidality, suicidal ideation, dysthymia or persistent depressive disorder, bipolar depressive disorder type I, bipolar depressive disorder type II, chronic pain, eating disorder NOS, pain disorder NOS, panic disorder, post-traumatic stress disorder, obsessive-compulsive disorder, complex regional pain syndrome, reflex sympathetic dystrophy, or any combination thereof.

12. The drug delivery device of claim 1, wherein the NMDA receptor antagonist is ketamine or a pharmaceutically acceptable salt thereof.

13. The drug delivery device of claim 1, wherein the NMDA receptor antagonist is an arylcyclohexylamine or arylcyclohexylamine derivative.

14. The drug delivery device of claim 1, wherein the at least one dosage regimen is configured by an authorized user who is a healthcare provider for the subject.

15. The drug delivery device of claim 1, wherein the at least one dosage regimen is prescribed for the subject by a healthcare provider.

16. The drug delivery device of claim 1, wherein the subject is not authorized to configure or modify the at least one dosage regimen.

17. The drug delivery device of claim 1, wherein the drug delivery device allows limited modification of the at least one dosage regimen by the subject.

18. The drug delivery device of claim 1, wherein the at least one dosage regimen comprises a plurality of dosing options selectable by the subject.

19. The drug delivery device of claim 18, wherein the plurality of dosing options is selected from the group consisting of bolus injection, continuous infusion.

20. The drug delivery device of claim 18, wherein the plurality of dosing options comprises differences in dosage size, dosage rate, infusion duration, or any combination thereof.

21. The drug delivery device of claim 1, further comprising a remote access module allowing an authorized user to remotely configure or modify the at least one dosage regimen over a network.

22. The drug delivery device of claim 1, further comprising a monitoring module allowing an authorized user to remotely monitor the at least one dosage regimen over a network.

23. The drug delivery device of claim 1, further comprising a communications module allowing the drug delivery device to pair with a communications device that provides a network connection for communicating with an authorized user.

24. The drug delivery device of claim 1, wherein the pump mechanism is configured to administer the drug formulation through subcutaneous or intramuscular injection.

25. The drug delivery device of claim 1, wherein the dose comprises an infusion rate of at least about 0.1 mg/hour.

26. The drug delivery device of claim 1, wherein the dosage regimen provides a clinically effective steady-state concentration of the NMDA receptor antagonist for at least 8 hours.

27. A system comprising:
a) a drug delivery device comprising a pump mechanism for administering a drug formulation comprising an NMDA receptor antagonist and a user interface allowing a subject to self-administer a dose of the drug formulation from a selection of at least one pre-programmed dosage regimen that is not configurable by the subject; and
b) a digital device of an authorized user in communication with the drug delivery device to allow the authorized user to configure, modify, or monitor the dosage regimen;
wherein the at least one dosage regimen provides an effective steady state drug plasma concentration while reducing side effects.

28. A method for self-treatment by a subject outside of a hospital or clinical setting, comprising:
a) obtaining a drug delivery device for administering a dose of a drug formulation comprising an NMDA receptor antagonist; and
b) self-administering the dose from a selection of at least one pre-programmed dosage regimen that is not configurable by the subject;
wherein the at least one dosage regimen provides an effective steady state drug plasma concentration while reducing side effects.

29. A pharmaceutical composition, comprising:
(i) an NMDA receptor antagonist, or a hydrate, solvate, or pharmaceutically acceptable salt thereof; and
(ii) at least one pharmaceutically acceptable excipient,
wherein the pharmaceutical composition is in a form for dosing or administration by intravenous (I.V.), intramuscular, subcutaneous, or intradermal injection.

30. The pharmaceutical composition of claim 29, wherein the at least one pharmaceutically acceptable excipient is (i) a surface-active agent, (ii) a non-ionic surfactant, (iii) a phospholipid solubilization agent, (iv) a cyclodextrin excipient, (v) an emulsion stabilizer, (vi) a preservative, (vii) an antimicrobial agent, or (viii) a topical analgesic.

31. A method for self-treatment by a subject outside of a hospital or clinical setting, comprising:
(i) obtaining a drug delivery device for administering a dose of the pharmaceutical composition of any one of claims 29-30; and
(ii) self-administering the dose from a selection of at least one pre-programmed dosage regimen that is not configurable by the subject;
wherein the at least one dosage regimen provides an effective steady state drug plasma concentration while reducing side effects.

32. A sealed reusable delivery and control system comprising drug delivery device of any of claims 1-26, wherein the drug delivery device further comprises:
a) a control system providing a multi-tiered security authentication configured to use one or more of a patient ID, physician ID, Delivery device ID, Mobile device ID, cartridge ID that is compared a database via security challenges;
b) an energy harvesting NFC tag for cartridge ID;
c) one or more accumulation registers; and
d) one or more tamper proof conductors.
